(19) United States
(12) Patent Application Publication
(10) Pub. No.: US 2010/0160301 A1
(43) Pub. Date: Jun. 24, 2010

(54) MICROANGIOPATHY TREATMENT AND PREVENTION

(75) Inventors: Elisabeth Perzborn, Wuppertal (DE); Frank Misselwitz, Heidelberg (DE)

Correspondence Address: CONNOLLY BOVE LODGE & HUTZ, LLP, P O BOX 2207, WILMINGTON, DE, 19899, US

(73) Assignee: BAYER HEALTHCARE AG, Leverkusen (unknown)

(21) Appl. No.: 12/089,650

(22) PCT Filed: Sep. 27, 2006
(86) PCT No.: PCT/EP2006/009373

§ 371 Date: Jul. 2, 2008

(30) Foreign Application Priority Data
Oct. 10, 2005 (DE) 102005048824.2

(51) Int. Cl. A61K 31/422; A61K 31/5377; A61K 31/541; A61K 31/4365; A61K 31/428; A61K 31/496; A61K 31/454; A61K 31/444; A61P 1/00; A61P 13/12; A61P 27/02

(52) U.S. Cl. 514/227.8; 514/236.8; 514/301; 514/367; 514/253.1; 514/254.02; 514/326; 514/333; 514/376

(57) ABSTRACT

The present invention relates to the use of selective factor Xa inhibitors, in particular of oxazolidinones of the formula (I)

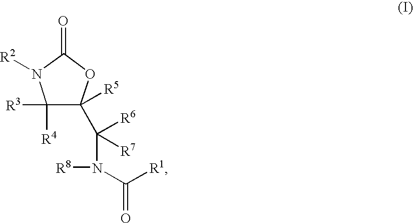

for the treatment and/or prophylaxis of microangiopathies and also their use for the production of medicaments for the treatment and/or prophylaxis of microangiopathies.

The present invention relates to the use of selective factor Xa inhibitors, in particular of oxazolidinones of the formula (I), for the treatment and/or prophylaxis of microangiopathies, and their use for the production of medicaments for the treatment and/or prophylaxis of microangiopathies.

Oxazolidinones of the formula (I) are known from WO 01/047919 and in particular act as selective inhibitors of blood clotting factor Xa and as anticoagulants.

Oxazolidinones of the formula (I) are selective factor Xa inhibitors and specifically inhibit only FXa. It was possible to demonstrate an antithrombotic action of factor Xa inhibitors in numerous animal models (cf. U. Sinha, P. Ku, J. Malinowski, B. Yan Zhu, R M. Scarborough, C K. Marlowe, P W. Wong, P. Hua Lin, S J. Hollenbach, Antithrombotic and hemostatic capacity of factor Xa versus thrombin inhibitors in models of venous and arteriovenous thrombosis, European Journal of Pharmacology 2000, 395, 51-59; A. Betz, Recent advances in Factor Xa inhibitors, Expert Opin. Ther. Patents 2001, 11, 1007; K. Tsong Tan, A. Makin, G. Y H Lip, Factor X inhibitors, Exp. Opin. Investig. Drugs 2003, 12, 799; J. Ruef, H A. Katus, New antithrombotic drugs on the horizon, Expert Opin. Investig. Drugs 2003, 12, 781; M M. Samama, Synthetic direct and indirect factor Xa inhibitors, Thrombosis Research 2002, 106, V267; M L. Quan, J M. Stnallheer, The race to an orally active Factor Xa inhibitor, Recent advances, J. Current Opinion in Drug Discovery & Development 2004, 7, 460-469) and in clinical studies on patients (The Ephesus Study, Blood 2000, 96, 490a; The Penthifra Study, Blood 2000, 96, 490a; The Pentamaks Study, Blood 2000, 96, 490a-491a; The Pentathlon 2000 Study, Blood 2000, 96, 491a). Factor Xa inhibitors can therefore preferably be employed in medicaments for the prophylaxis and/or treatment of thromboembolic diseases. Selective FXa inhibitors show a broad therapeutic window. In numerous animal experimental investigations, it was possible to show that FXa inhibitors show an antithrombotic action in models of thrombosis without, or only slightly with, a prolonging action on bleeding times (cf. R J Leadly, Coagulation factor Xa inhibition: biological background and rationale, Curr Top Med Chem 2001; 1, 151-159). An individual dose in the case of anticoagulation with selective FXa inhibitors is therefore not necessary.

Microangiopathies are a syndrome caused by stenosis and thrombosis of small and very small vessels. Frequent causes of microangiopathies are embolizing microthrombi of proximal vessels, endothelial damage with overshooting activation of platelets and clotting. Thus, in the pathogenesis of microangiopathy, endothelial defects are a crucial pathophysiological substrate. The normal, intact endothelial lining of the blood vessels is athrombogenic. In the case of injuries, thrombogenic properties of the endothelium become predominant. Resulting thrombi lead to microangiopathic hemolysis, to the occlusion of small vessels and to organ ischemia.

It has now surprisingly been found that selective factor Xa inhibitors, in particular oxazolidinones of the formula (I), are also suitable for the treatment and prevention of microangiopathies.

The present invention relates to the use of selective factor Xa inhibitors for the production of medicaments for the treatment and/or prophylaxis of microangiopathies.

The present invention relates in particular to the use of compounds of the formula (I)

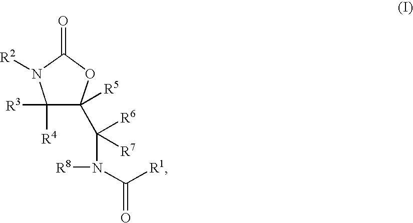

in which:
R¹ represents optionally benzo-fused thiophene (thienyl), which can optionally be mono- or polysubstituted;
R² represents any desired organic radical;
R³, R⁴, R⁵, R⁶, R⁷ and R⁸ are identical or different and represent hydrogen or (C₁-C₆)-alkyl
   and their salts, solvates and solvates of the salts for the production of medicaments for the treatment and/or prophylaxis of microangiopathies.

The use is preferred here of compounds of the formula (I),

in which
R¹ represents optionally benzo-fused thiophene (thienyl), which can optionally be mono- or polysubstituted by a radical from the group consisting of halogen; cyano; nitro; amino; aminomethyl; (C₁-C₈)-alkyl, which for its part can optionally be mono- or polysubstituted by halogen; (C₃-C₇)-cycloalkyl; (C₁-C₈)-alkoxy; imidazolinyl; —C(═NH)NH₂; carbamoyl; and mono- and di-(C₁-C₄)-alkylaminocarbonyl,
R² represents one of the following groups:
   A-,
   A-M-,
   D-M-A-,
   B-M-A-,
   B—,
   B-M-,
   B-M-B—,
   D-M-B—,
   where:
   the radical “A” represents (C₆-C₁₄)-aryl, preferably (C₆-C₁₀)-aryl, in particular phenyl or naphthyl, very particularly preferably phenyl;
   the radical “B” represents a 5- or 6-membered aromatic heterocycle, which contains up to 3 heteroatoms and/or hetero chain members, in particular up to 2 heteroatoms and/or hetero chain members, from the series consisting of S, N, NO(N-oxide) and O;
   the radical “D” represents a saturated or partially unsaturated, mono- or bicycle, optionally benzo-fused 4- to 9-membered heterocycle, which contains up to three heteroatoms and/or hetero chain members from the series consisting of S, SO, SO₂, N, NO (N-oxide) and O;
   the radical “M” represents —NH—, —CH₂—, —CH₂CH₂—, —O—, —NH—CH₂—, —CH₂—NH—, —OCH₂—, —CH₂O—, —CONH—, —NHCO—, —COO—, —OOC—, —S—, —SO₂— or a covalent bond;
   where
   the previously defined groups “A”, “B” and “D” can in each case be optionally mono- or polysubstituted by a radical from the group consisting of halogen; trifluoromethyl; oxo; cyano; nitro; carbamoyl; pyridyl; (C₁-C₆)-alkanoyl; (C₃-C₇)-cycloalkanoyl; (C₆-C₁₄)-arylcarbonyl; (C₅-C₁₀)-heteroarylcarbonyl; (C₁-C₆)-alkanoyloxymethyloxy; (C₁-C₄)-hydroxyalkylcarbonyl; —COOR²⁷; —SO₂R²⁷; —C(NR²⁷R²⁸)═NR²⁹; —CONR²⁸R²⁹; —SO₂NR²⁸R²⁹; —OR³⁰; —NR³⁰R³¹, (C₁-C₆)-alkyl and (C₃-C₇)-cycloalkyl,
   where (C₁-C₆)-alkyl and (C₃-C₇)-cycloalkyl for their part can optionally be substituted by a radical from the group consisting of cyano; —OR²⁷; —NR²⁸SR²⁹; —CO(NH)ᵥ(NR²⁷R²⁸) and —C(NR²⁷R²⁸)═NR²⁹,
   where:
   v is either 0 or 1 and
   R²⁷, R²⁸ and R²⁹ are identical or different and independently of one another are hydrogen, (C₁-C₄)-alkyl, (C₃-C₇)-cycloalkyl, (C₁-C₄)-alkanoyl, carbamoyl, trifluoromethyl, phenyl or pyridyl,
   and/or
   R²⁷ and R²⁸ or R²⁷ and R²⁹, together with the nitrogen atom to which they are bonded, form a saturated or partially unsaturated 5- to 7-membered heterocycle having up to three, preferably up to two, identical or different heteroatoms from the group consisting of N, O and S, and
   R³⁰ and R³¹ are identical or different and independently of one another are hydrogen, (C₁-C₄)-alkyl, (C₃-C₇)-cycloalkyl, (C₁-C₄)-alkylsulfonyl, (C₁-C₄)-hydroxyalkyl, (C₁-C₄)-aminoalkyl, di-(C₁-C₄)-alkylamino-(C₁-C₄)-alkyl, —CH₂C(NR²⁷R²⁸)═NR²⁹ or —COR³³,
      where
      R³³ is (C₁-C₆)-alkoxy, (C₁-C₄)-alkoxy-(C₁-C₄)-alkyl, (C₁-C₄)-alkoxycarbonyl-(C₁-C₄)-alkyl, (C₁-C₄)-aminoalkyl, (C₁-C₄)-alkoxycarbonyl, (C₁-C₄)-alkanoyl-(C₁-C₄)-alkyl, (C₃-C₇)-cycloalkyl, (C₂-C₆)-alkenyl, (C₁-C₈)-alkyl, which can optionally be substituted by phenyl or acetyl, (C₆-C₁₄)-aryl, (C₅-C₁₀)-heteroaryl, trifluoromethyl, tetrahydrofuranyl or butyrolactone,
R³, R⁴, R⁵, R⁶, R⁷ and R⁸ are identical or different and represent hydrogen or (C₁-C₆)-alkyl
   and their salts, solvates and solvates of the salts.

Likewise preferred here is the use of compounds of the general formula (I),

in which
R¹ represents thiophene (thienyl), in particular 2-thiophene, which can optionally be mono- or polysubstituted by halogen, preferably chlorine or bromine, amino, aminomethyl or (C₁-C₈)-alkyl, preferably methyl, where the (C₁-C₈)-alkyl radical can for its part optionally be mono- or polysubstituted by halogen, preferably fluorine,
R² represents one of the following groups:
   A-,
   A-M-,
   D-M-A-,
   B-M-A-,
   B—,
   B-M-,
   B-M-B—,
   D-M-B—,
   where:
   the radical “A” represents (C₆-C₁₄)-aryl, preferably (C₆-C₁₀)-aryl, in particular phenyl or naphthyl, very particularly preferably phenyl;
   the radical “B” represents a 5- or 6-membered aromatic heterocycle, which contains up to 3 heteroatoms and/or hetero chain members, in particular up to 2 heteroatoms and/or hetero chain members, from the series S, N, NO(N-oxide) and O;
   the radical “D” represents a saturated or partially unsaturated 4- to 7-membered heterocycle, which contains up to three heteroatoms and/or hetero chain members from the series S, SO, SO₂, N, NO(N-oxide) and O;
   the radical “M” represents —NH—, —CH₂—, —CH₂CH₂—, —O—, —NH—CH₂—, —CH₂—NH—, —CH₂O—, —CONH—, —NHCO—, —COO—, —OOC—, —S— or a covalent bond;
   where
   the previously defined groups “A”, “B” and “D” can in each case be optionally mono- or polysubstituted by a radical from the group consisting of halogen; trifluoromethyl; oxo; cyano; nitro; carbamoyl; pyridyl; (C₁-C₆)-alkanoyl; (C₃-C₇)-cycloalkanoyl; (C₆-C₁₄)-arylcarbonyl; (C₅-C₁₀)-heteroarylcarbonyl; (C₁-C₆)-alkanoyloxymethyloxy; —COOR²⁷; —SO₂R²⁷; —C(NR²⁷R²⁸)═NR²⁹; —CONR²⁸R²⁹; —SO₂NR²⁸R²⁹; —OR³⁶; —NR³⁰R³¹, (C₁-C₆)-alkyl and (C₃-C₇)-cycloalkyl,
   where (C₁-C₆)-alkyl and (C₃-C₇)-cycloalkyl for their part can optionally be substituted by a radical from the group consisting of cyano; —OR²⁷; —NR²⁸R²⁹; —CO(NH)ᵥ(NR²⁷R²⁸) and —C(NR²⁷R²⁸)NR²⁹,
   where:
   v is either 0 or 1 and
   R²⁷, R²⁸ and R²⁹ are identical or different and independently of one another are hydrogen, (C₁-C₄)-alkyl or (C₃-C₇)-cycloalkyl,
   and/or
   R²⁷ and R²⁸ or R²⁷ and R²⁹, together with the nitrogen atom to which they are bonded, form a saturated or partially unsaturated 5- to 7-membered heterocycle having up to three, preferably up to two identical or different heteroatoms from the group consisting of N, O and S, and
   R³⁹ and R³¹ are identical or different and independently of one another are hydrogen, (C₁-C₄)-alkyl, (C₃-C₇)-cycloalkyl, (C₁-C₄)-alkylsulfonyl, (C₁-C₄)-hydroxyalkyl, (C₁-C₄)-aminoalkyl, di-(C₁-C₄)-alkylamino-(C₁-C₄)-alkyl, (C₁-C₄)-alkanoyl, (C₆-C₁₄)-arylcarbonyl, (C₅-C₁₀)-heteroarylcarbonyl, (C₁-C₄)-allylaminocarbonyl or —CH₂C(NR²⁷R²⁸)═NR²⁹,
R³, R⁴, R⁵, R⁶, R⁷ and R⁸ are identical or different and represent hydrogen or (C₁-C₆)-alkyl
   and their salts, solvates and solvates of the salts.

Particularly preferred here is the use of compounds of the general formula (I),

in which
R¹ represents thiophene (thienyl), in particular 2-thiophene, which can optionally be mono- or polysubstituted by halogen, preferably chlorine or bromine, or (C₁-C₈)-alkyl, preferably methyl, where the (C₁-C₈)-alkyl radical can for its part optionally be mono- or polysubstituted by halogen, preferably fluorine,
R² represents one of the following groups:
   A-,
   A-M-,
   D-M-A-,
   B-M-A-,
   B—,
   B-M-,
   B-M-B—,
   D-M-B—,
   where:
   the radical “A” represents phenyl or naphthyl, in particular phenyl;
   the radical “B” represents a 5- or 6-membered aromatic heterocycle, which contains up to 2 heteroatoms from the series S, N, NO(N-oxide) and O;
   the radical “D” represents a saturated or partially unsaturated 5- or 6-membered heterocycle, which contains up to two heteroatoms and/or hetero chain members from the series S, SO, SO₂, N, NO(N-oxide) and O;
   the radical “M” represents —NH—, —O—, —NH—CH₂—, —CH₂—NH—, —OCH₂—, —CH₂O—, —CONH—, —NHCO— or a covalent bond;
   where
   the previously defined groups “A”, “B” and “D” can in each case optionally be mono- or polysubstituted by a radical from the group consisting of halogen; trifluoromethyl; oxo; cyano; pyridyl; (C₁-C₃)-alkanoyl; (C₆-C₁₀)-arylcarbonyl; (C₅-C₆)-heteroarylcarbonyl; (C₁-C₃)-alkanoyloxymethyloxy; —C(NR²⁷R²⁸)═NR²⁹; —CONR²⁸R²⁹; —SO₂NR²⁸R²⁹; —OH; —NR³⁰R³¹; (C₁-C₄)-alkyl; and cyclopropyl, cyclopentyl or cyclohexyl,
   where (C₁-C₄)-alkyl and cyclopropyl, cyclopentyl or cyclohexyl for their part can optionally be substituted by a radical from the group consisting of cyano; —OH; —OCH₃; —NR²⁸R²⁹; —CO(NH)ᵥ(NR²⁷R²⁸) and —C(NR²⁷R²⁸)═NR²⁹,
   where:
   v is either 0 or 1, preferably 0, and
   R²⁷, R²⁸ and R²⁹ are identical or different and independently of one another are hydrogen, (C₁-C₄)-alkyl or else cyclopropyl, cyclopentyl or cyclohexyl
   and/or
   R²⁷ and R²⁸ or R²⁷ and R²⁹, together with the nitrogen atom to which they are bonded, can form a saturated or partially unsaturated 5- to 7-membered heterocycle having up to two identical or different heteroatoms from the group consisting of N, O and S, and
   R³⁰ and R³¹ are identical or different and independently of one another are hydrogen, (C₁-C₄)-alkyl, cyclopropyl, cyclopentyl, cyclohexyl, (C₁-C₄)-alkylsulfonyl, (C₁-C₄)-hydroxyalkyl, (C₁-C₄)-aminoalkyl, di-(C₁-C₄)-alkylamino-(C₁-C₄)-alkyl, (C₁-C₃)-alkanoyl or phenylcarbonyl,
R³, R⁴, R⁵, R⁶, R⁷ and R⁸ are identical or different and represent hydrogen or (C₁-C₆)-alkyl
   and their salts, solvates and solvates of the salts.

Preferred in particular here is the use of compounds of the general formula (I),

in which
R¹ represents 2-thiophene, which can optionally be substituted in the 5-position by a radical from the group consisting of chlorine, bromine, methyl or trifluoromethyl,
R² represents one of the following groups:
   A-,
   A-M-,
   D-M-A-,
   B-M-A-,
   B—,
   B-M-,
   B-M-B—,
   D-M-B—,
   where:
   the radical “A” represents phenyl or naphthyl, in particular phenyl;
   the radical “B” represents a 5- or 6-membered aromatic heterocycle, which contains up to 2 heteroatoms from the series S, N, NO(N-oxide) and O;
   the radical “D” represents a saturated or partially unsaturated 5- or 6-membered heterocycle, which contains a nitrogen atom and optionally a further heteroatom and/or hetero chain member from the series S, SO, SO₂ and O; or up to two heteroatoms and/or hetero chain members from the series S, SO, SO₂ and O;
   the radical “M” represents —NH—, —O—, —NH—CH₂—, —CH₂—NH—, —OCH₂—, —CH₂O—, —CONH—, —NHCO— or a covalent bond;
   where
   the previously defined groups “A”, “B” and “D” can in each case optionally be mono- or polysubstituted by a radical from the group consisting of halogen; trifluoromethyl; oxo; cyano; pyridyl; (C₁-C₃)-alkanoyl; (C₆-C₁₀)-arylcarbonyl; (C₅-C₆)-heteroarylcarbonyl; (C₁-C₃)-alkanoyloxymethyloxy; —CONR²⁸R²⁹; —SO₂NR²⁸R²⁹; —OH; —NR³⁰R³¹; (C₁-C₄)-alkyl; and cyclopropyl, cyclopentyl or cyclohexyl,
   where (C₁-C₄)-alkyl and cyclopropyl, cyclopentyl or cyclohexyl can for their part optionally be substituted by a radical from the group consisting of cyano; —OH; —OCH₃; —NR²⁸R²⁹; —CO(NH)ᵥ(NR²⁷R²⁸) and —C(NR²⁷R²⁸)═NR²⁹,
   where:
   v is either 0 or 1, preferably 0, and
   R²⁷, R²⁸ and R²⁹ are identical or different and independently of one another are hydrogen, (C₁-C₄)-alkyl or else cyclopropyl, cyclopentyl or cyclohexyl
   and/or
   R²⁷ and R²⁸ or R²⁷ and R²⁹, together with the nitrogen atom to which they are bonded, can form a saturated or partially unsaturated 5- to 7-membered heterocycle having up to two identical or different heteroatoms from the group consisting of N, O and S and
   R³⁰ and R³¹ are identical or different and independently of one another are hydrogen, (C₁-C₄)-alkyl, cyclopropyl, cyclopentyl, cyclohexyl, (C₁-C₄)-alkylsulfonyl, (C₁-C₄)-hydroxyalkyl, (C₁-C₄)-aminoalkyl, di-(C₁-C₄)-alkylamino-(C₁-C₄)-alkyl, (C₁-C₃)-alkanoyl or phenylcarbonyl,
R³, R⁴, R⁵, R⁶, R⁷ and R⁸ are identical or different and represent hydrogen or (C₁-C₄)-alkyl
   and their salts, solvates and solvates of the salts.

Very particularly preferred here is the use of compounds of the general formula (I),

in which
R¹ represents 2-thiophene, which is substituted in the 5-position by a radical from the group consisting of chlorine, bromine, methyl or trifluoromethyl,
R² represents D-A-:
   where:
   the radical “A” represents phenylene;
   the radical “D” represents a saturated 5- or 6-membered heterocycle,
   which is linked to “A” via a nitrogen atom,
   which in direct vicinity to the linking nitrogen atom has a carbonyl group and
   in which a ring carbon member can be replaced by a heteroatom from the series S, N and O;
   where
   the previously defined group “A” can optionally be mono- or disubstituted in the meta-position with respect to the linkage to the oxazolidinone by a radical from the group consisting of fluorine, chlorine, nitro, amino, trifluoromethyl, methyl or cyano,
R⁸, R⁴, R⁵, R⁶, R⁷ and R⁸ represent hydrogen
   and their salts, solvates and solvates of the salts.

Likewise very particularly preferred here is the use of the compound having the following formula

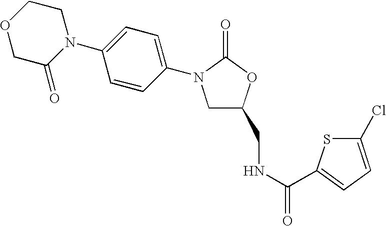

and its salts, solvates and solvates of the salts.

Oxazolidinones were originally described essentially only as antibiotics, sporadically also as MAO inhibitors and fibrinogen antagonists (survey: Riedl, B., Enderrnann, R., Exp. Opin. Ther. Patents 1999, 9 (5), 625), a small 5-[acylaminomethyl] group (preferably 5-[acetylaminomethyl]) appearing to be essential for the antibacterial activity.

Substituted aryl- and heteroarylphenyloxazolidinones, in which a mono- or polysubstituted phenyl radical can be bonded to the N atom of the oxazolidinone ring and which can contain an unsubstituted N-methyl-2-thiophenecarboxamide radical in the 5-position of the oxazolidinone ring, and their use as antibacterially active substances are known from the U.S. patent specifications U.S. Pat. No. 5,929,248, U.S. Pat. No. 5,801,246, U.S. Pat. No. 5,756,732, U.S. Pat. No. 5,654,435, U.S. Pat. No. 5,654,428 and U.S. Pat. No. 5,565,571.

Moreover, benzamidine-containing oxazolidinones are known as synthetic intermediates in the synthesis of factor Xa inhibitors or fibrinogen antagonists (WO 99/31092, EP 0 623 615).

Compounds which can be used according to the invention, also described below as compounds according to the invention, are the compounds of the formula (I) and their salts, solvates and solvates of the salts, the compounds comprised by formula (I) of the formulae mentioned below and their salts, solvates and solvates of the salts, and the compounds comprised by formula (I), mentioned below as working examples, and their salts, solvates and solvates of the salts, inasmuch as the compounds comprised by formula (I) and mentioned below are not already salts, solvates and solvates of the salts.

Depending on their structure, the compounds according to the invention can exist in stereoisomeric forms (enantiomers, diastereomers). The invention therefore comprises the use of the enantiomers or diastereomers and their respective mixtures.

If the compounds according to the invention can occur in tautomeric forms, the present invention comprises the use of all tautomeric forms.

Preferred salts in the context of the present invention are physiologically acceptable salts of the compounds according to the invention. Also included are salts which are not suitable themselves for pharmaceutical applications, but can be used, for example, for the isolation or purification of the compounds according to the invention.

Physiologically acceptable salts of the compounds according to the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases, such as, by way of example and preferably, alkali metal salts (e.g. sodium and potassium salts), alkaline earth metal salts (e.g. calcium and magnesium salts) and ammonium salts, derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanoI, procaine, dibenzylamine, N-methylmorpholine, arginine, lysine, ethylenediamine and N-methylpiperidine.

Solvates are designated within the context of the invention as those forms of the compounds according to the invention which form a complex in the solid or liquid state by coordination with solvent molecules. Hydrates are a special form of solvates, in which the coordination takes place with water. Solvates which are preferred in the context of the present invention are hydrates.

Moreover, the present invention also comprises the use of prodrugs of the compounds according to the invention. The term “prodrugs” comprises compounds which can be biologically active or inactive themselves, but during their residence time in the body are converted to give compounds according to the invention (for example metabolically or hydrolytically).

In the context of the present invention, the substituents, if not specified otherwise, have the following meaning:

Halogen represents fluorine, chlorine, bromine and iodine. Chlorine and fluorine are preferred.

(C₁-C₈)-Alkyl represents a straight-chain or branched alkyl radical having 1 to 8 carbon atoms. Examples which may be mentioned are: methyl, ethyl, n-propyl, isopropyl, n-butyl, isobutyl, tert-butyl, n-pentyl and n-hexyl. The corresponding alkyl groups having fewer carbon atoms such as, for example, (C₁-C₆)-alkyl and (C₁-C₄)-alkyl are analogously derived from this definition. In general, it applies that (C₁-C₄)-alkyl is preferred.

The meaning of the corresponding constituent of other more complex substituents is also derived from this definition such as, for example, in the case of alkylsulfonyl, hydroxyalkyl, hydroxyalkylcarbonyl, alkoxyalkyl, alkoxycarbonylalkyl, alkanoylalkyl, aminoalkyl or alkylamino-alkyl.

(C₃-C₇)-Cycloalkyl represents a cyclic alkyl radical having 3 to 7 carbon atoms. Examples which may be mentioned are: cyclopropyl, cyclobutyl, cyclopentyl, cyclohexyl or cycloheptyl. The corresponding cycloalkyl groups having fewer carbon atoms such as, for example, (C₃-C₅)-cycloalkyl are analogously derived from this definition. Cyclopropyl, cyclopentyl and cyclohexyl are preferred.

The meaning of the corresponding constituent of other more complex substituents such as, for example, cycloalkanoyl is also derived from this definition.

(C₇-C₆)-Alkenyl represents a straight-chain or branched alkenyl radical having 2 to 6 carbon atoms. A straight-chain or branched alkenyl radical having 2 to 4 carbon atoms is preferred. Examples which may be mentioned are: vinyl, allyl, isopropenyl and n-but-2-en-1-yl.

(C₁-C₈)-Alkoxy represents a straight-chain or branched alkoxy radical having 1 to 8 carbon atoms. Examples which may be mentioned are: methoxy, ethoxy, n-propoxy, isopropoxy, n-butoxy, isobutoxy, tert-butoxy, n-pentoxy, n-hexoxy, n-heptoxy and n-octoxy. The corresponding alkoxy groups having fewer carbon atoms such as, for example, (C₁-C₆)-alkoxy and (C₁-C₄)-alkoxy are analogously derived from this definition. In general, it applies that (C₁-C₄)-alkoxy is preferred.

The meaning of the corresponding constituent of other more complex substituents such as, for example, alkoxyalkyl, alkoxycarbonylalkyl and alkoxycarbonyl is also derived from this definition.

Mono- or di-(C₁-C₄)-alkylaminocarbonyl represents an amino group which is linked via a carbonyl group and which contains a straight-chain or branched or two identical or different straight-chain or branched alkyl substituents in each case having 1 to 4 carbon atoms. Examples which may be mentioned are: methylamino, ethylamino, n-propylamino, isopropylamino, t-butylamino, N,N-dimethylamino, N,N-diethylamino, N-ethyl-N-methylamino, N-methyl-N-n-propylamino, N-iso-propyl-N-n-propylamino and N-t-butyl-N-methylamino.

(C₁-C₄)-Alkanoyl represents a straight-chain or branched alkyl radical having 1 to 6 carbon atoms, which carries a double-bonded oxygen atom in the 1-position and is linked via the 1-position. Examples which may be mentioned are: formyl, acetyl, propionyl, n-butyryl, i-butyryl, pivaloyl, n-hexanoyl. The corresponding alkanoyl groups having fewer carbon atoms such as, for example, (C₁-C₅)-alkanoyl, (C₁-C₄)-alkanoyl and (C₁-C₃)-alkanoyl are analogously derived from this definition. In general, it applies that (C₁-C₃)-alkanoyl is preferred.

The meaning of the corresponding constituent of other more complex substituents such as, for example, cycloalkanoyl and alkanoylalkyl is also derived from this definition.

(C₁-C₇)-Cycloalkanoyl represents a cycloalkyl radical as defined previously having 3 to 7 carbon atoms, which is linked via a carbonyl group.

(C₁-C₆)-Alkanoyloxymethyloxy represents a straight-chain or branched alkanoyloxymethyloxy radical having 1 to 6 carbon atoms. Examples which may be mentioned are: acetoxymethyloxy, propionoxymethyloxy, n-butyroxymethyloxy, i-butyroxymethyloxy, pivaloyloxymethyloxy, n-hexanoyloxymethyloxy. The corresponding alkanoyloxymethyloxy groups having fewer carbon atoms such as, for example, (C₁-C₃)-alkanoyloxymethyloxy are analogously derived from this definition. In general, it applies that (C₁-C₃)-alkanoyloxymethyloxy is preferred.

(C₆-C₁₄)-Aryl represents an aromatic radical having 6 to 14 carbon atoms. Examples which may be mentioned are: phenyl, naphthyl, phenanthrenyl and anthracenyl. The corresponding aryl groups having fewer carbon atoms such as, for example, (C₆-C₁₀)-aryl are analogously derived from this definition. In general, it applies that (C₆-C₁₀)-aryl is preferred.

The meaning of the corresponding constituent of other more complex substituents such as, for example, arylcarbonyl is also derived from this definition.

(C₅-C₁₀)-Heteroaryl or a 5- to 10-membered aromatic heterocycle having up to 3 heteroatoms and/or hetero chain members from the series S, O, N and/or NO(N-oxide) represents a mono- or bicyclic heteroaromatic, which is linked via a ring carbon atom of the heteroaromatic, optionally also via a ring nitrogen atom of the heteroaromatic. Examples which may be mentioned are pyridyl, pyridyl N-oxide, pyrimidyl, pyridazinyl, pyrazinyl, thienyl, furyl, pyrrolyl, pyrazolyl, imidazolyl, thiazolyl, oxazolyl or isoxazolyl, indolizinyl, indolyl, benzo[b]thienyl, benzo[b]furyl, indazolyl, quinolyl, isoquinolyl, naphthyridinyl, quinazolinyl. The corresponding heterocycles having a smaller ring size such as, for example, 5- or 6-membered aromatic heterocycles are analogously derived from this definition. In general, it applies that 5- or 6-membered aromatic heterocycles such as, for example, pyridyl, pyridyl N-oxide, pyrimidyl, pyridazinyl, furyl and thienyl are preferred.

The meaning of the corresponding constituent of other more complex substituents such as, for example, (C₅-C₁₀)-heteroarylcarbonyl is also derived from this definition.

A 3- to 9-membered saturated or partially unsaturated, mono- or bicyclic, optionally benzo-fused heterocycle having up to 3 heteroatoms and/or hetero chain members from the series S, SO, SO₂, N, NO (N-oxide) and/or O represents a heterocycle which can contain one or more double bonds, which can be mono- or bicyclic, in which a benzene ring can be fused onto two adjacent ring carbon atoms, and which is linked via a ring carbon atom or a ring nitrogen atom. Examples which may be mentioned are: tetrahydrofuryl, pyrrolidinyl, pyrrolinyl, piperidinyl, 1,2-dihydropyridinyl, 1,4-dihydropyridinyl, piperazinyl, morpholinyl, morpholinyl N-oxide, thiomorpholinyl, azepinyl, 1,4-diazepinyl and cyclohexyl. Piperidinyl, morpholinyl and pyrrolidinyl are preferred.

The corresponding cycles having a smaller ring size such as, for example, 5- to 7-membered cycles are derived analogously from this definition.

The compounds of the formula (I) can be prepared by either, according to a process alternative
[A], reacting compounds of the general formula (II)

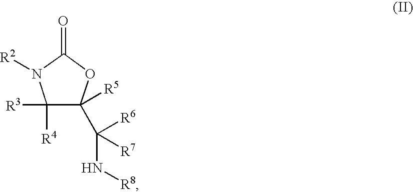

in which
the radicals R², R³, R⁴, R⁵, R⁶, R⁷ and R⁸ have the meanings indicated above,
with carboxylic acids of the general formula (III)

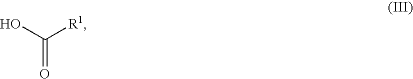

in which
the radical R¹ has the meaning indicated above,
or else with the corresponding carbonyl halides, preferably carbonyl chlorides, or else with the corresponding symmetrical or mixed carboxylic anhydrides of the previously defined carboxylic acids of the general formula (III)
in inert solvents, optionally in the presence of an activating or coupling reagent and/or a base, to give compounds of the general formula (I)

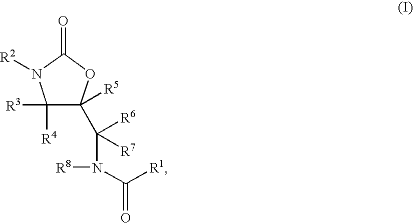

in which
the radicals R¹, R², R³, R⁴, R⁵, R⁶, R⁷ and R⁸ have the meanings indicated above,
or else, according to a process alternative
[B], converting compounds of the general formula (IV)

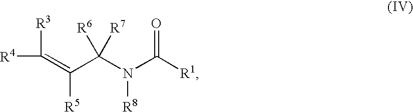

in which
the radicals R¹, R³, R⁴, R⁵, R⁶, R⁷ and R⁸ have the meanings indicated above,
using a suitable selective oxidant in an inert solvent, to the corresponding epoxide of the general formula (V)

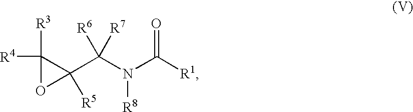

in which
the radicals R¹, R³, R⁴, R⁵, R⁶, R⁷ and R⁸ have the meanings indicated above,
and by reaction in an inert solvent, optionally in the presence of a catalyst, with an amine of the general formula (VI)

R²—NH₂ (VI),
in which
the radical R² has the meaning indicated above,
first preparing the compounds of the general formula (VII)

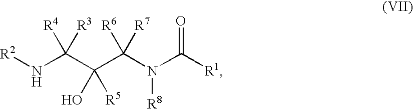

in which
the radicals R¹, R², R³, R⁴, R⁵, R⁶, R⁷ and R⁸ have the meanings indicated above,
and
subsequently, in an inert solvent in the presence of phosgene or phosgene equivalents such as, for example, carbonyldiimidazole (CDI), cyclizing them to the compounds of the general formula (I)

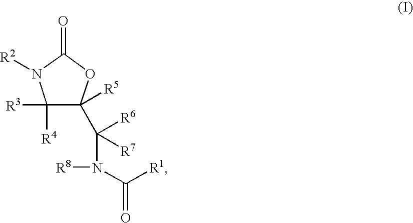

in which
the radicals R¹, R², R³, R⁴, R⁵, R⁶, R⁷ and R⁸ have the meanings indicated above,
where both for process alternative [A] and for process alternative [B] for the case where R² contains a 3- to 7-membered saturated or partially unsaturated cyclic hydrocarbon radical having one or more identical or different heteroatoms from the group consisting of N and S, an oxidation with a selective oxidant to give the corresponding sulfone, sulfoxide or N-oxide can follow
and/or
where both for process alternative [A] and for process alternative [B] for the case where the compound prepared in this way contains a cyano group in the molecule, an amidination of this cyano group using the customary methods can follow
and/or
where both for process alternative [A] and for process alternative [B] for the case where the compound prepared in this way contains a BOC amino-protective group in the molecule, a cleavage of this BOC amino-protective group can follow using the customary methods
and/or
where both for process alternative [A] and for process alternative [B] for the case where the compound prepared in this way contains an aniline or benzylamine radical in the molecule, a reaction of this amino group with different reagents such as carboxylic acids, carboxylic anhydrides, carbonyl chlorides, isocyanates, sulfonyl chlorides or alkyl halides can follow to give the corresponding derivatives
and/or
where both for process alternative [A] and for process alternative [B] for the case where the compound prepared in this way contains a phenyl ring in the molecule, a reaction with chlorosulfonic acid and subsequent reaction with amines to give the corresponding sulfonamides can follow.

The processes can be illustrated by way of example by the following reaction schemes:

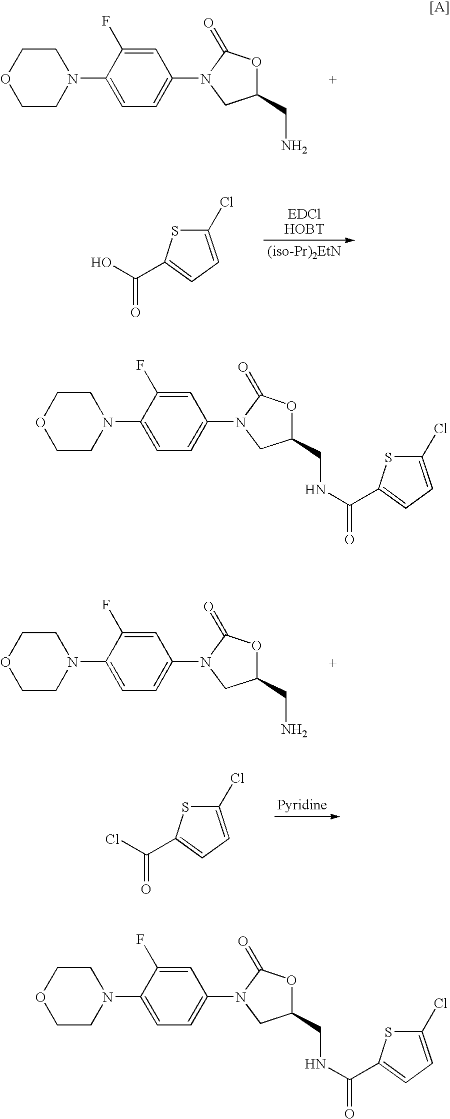

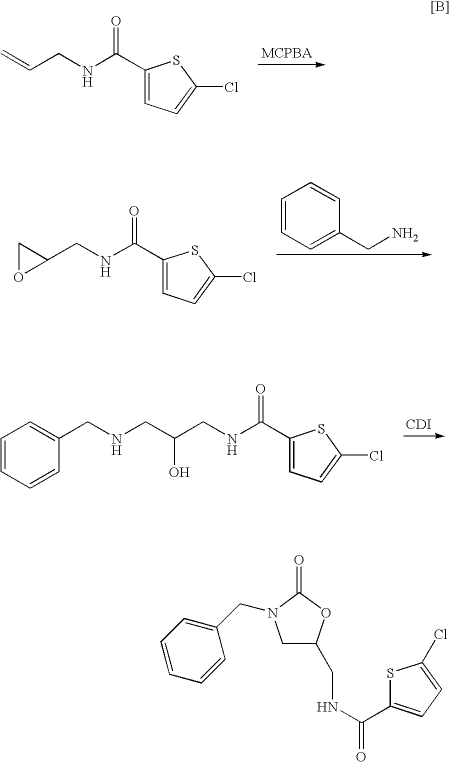

The previously described oxidation step optionally carried out can be illustrated by way of example by the following reaction schemes:

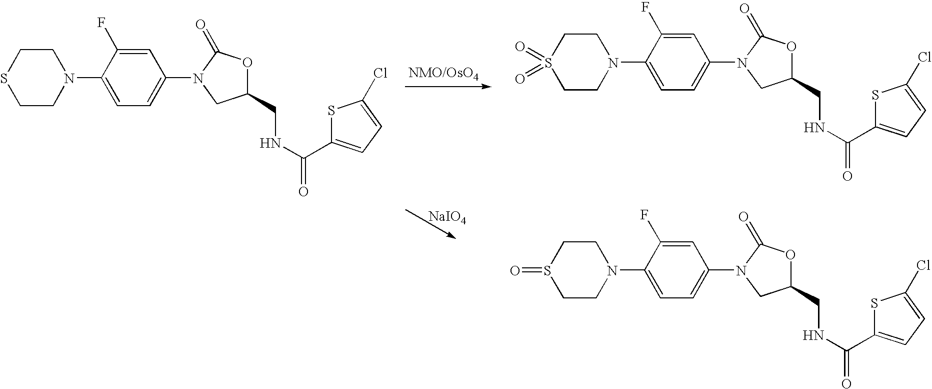

Suitable solvents for the previously described processes here are organic solvents which are inert under the reaction conditions. These include halogenohydrocarbons such as dichloromethane, trichloromethane, tetrachloromethane, 1,2-dichloroethane, trichloroethane, tetrachloroethane, 1,2-dichloroethylene or trichloroethylene, ethers such as diethyl ether, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane or cyclohexane, dimethylformamide, dimethyl sulfoxide, acetonitrile, pyridine, hexa-methylphosphoramide or water.

It is likewise possible to employ solvent mixtures of the previously mentioned solvents.

Suitable activating or coupling reagents for the previously described processes here are the reagents customarily used for this purpose, for example N′-(3-dimethylaminopropyl)-N-ethylcarbodiimide.HCl, N,N′-dicyclohexylcarbodiimide, 1-hydroxy-1H-benzotriazole.H₂O and the like.

Suitable bases are the customary inorganic or organic bases. These preferably include alkali metal hydroxides such as, for example, sodium hydroxide or potassium hydroxide or alkali metal carbonates such as sodium carbonate or potassium carbonate or sodium methoxide or potassium methoxide or sodium ethoxide or potassium ethoxide or potassium tert-butoxide or amides such as sodium amide, lithium bis(trimethylsilyl)amide or lithium diisopropylamide or amines such as triethylamine, diisopropylethylamine, diisopropylamine, 4-N,N-dimethylaminopyridine or pyridine.

The base can be employed here in an amount from 1 to 5 mol, preferably from 1 to 2 mol, based on 1 mol of the compounds of the general formula (II).

The reactions are in general carried out in a temperature range from −78° C. up to reflux temperature, preferably in the range from 0° C. to reflux temperature.

The reactions can be carried out at normal, elevated or reduced pressure (e.g. in the range from 0.5 to 5 bar). In general, they are carried out at normal pressure.

Possible suitable selective oxidants both for the preparation of the epoxides and for the oxidation optionally carried out to give the sulfone, sulfoxide or N-oxide are, for example, m-chloro-perbenzoic acid (MCPBA), sodium metaperiodate, N-methylmorpholine N-oxide (NMO), monoperoxyphthalic acid or osmium tetroxide.

With respect to the preparation of the epoxides, the preparation conditions customary for this purpose are used.

With respect to the more detailed process conditions for the oxidation optionally carried out to give the sulfone, sulfoxide or N-oxide, reference can be made to the following literature: M. R. Barbachyn et al., J. Med. Chem. 1996, 39, 680 and also WO 97/10223.

In addition, reference is made to Examples 14 to 16 presented in the experimental section.

The optionally carried out amidination is carried out under the customary conditions. For further details, reference can be made to Examples 31 to 35 and 140 to 147.

The compounds of the formulae (II), (III), (IV) and (VI) are known per se to the person skilled in the art or can be prepared according to customary methods. For oxazolidinones, in particular the 5-(aminomethyl)-2-oxooxazolidines needed, cf. WO 98/01446; WO 93/23384; WO 97/03072; J. A. Tucker et al., J. Med. Chem. 1998, 41, 3727; S. J. Brickner et al., J. Med. Chem. 1996, 39, 673; W. A. Gregory et al., J. Med. Chem. 1989, 32, 1673.

A preferred compound of the formula (I) which can be used according to the invention is 5-chloro-N-({(5S)-2-oxo-3-[4-(3-oxo-4-morpholinyl)phenyl]-1,3-oxazolidin-5-yl}methyl)-2-thiophene carboxamide, the compound from Example 44.

The term microangiopathies in the sense of the present invention comprises occlusive syndromes, which especially result on the skin and other organs.

The term microangiopathies further comprises the primary forms of thrombotic microangiopathies (TMA), such as thrombotic thrombocytopenic purpura (TTP) and the hemolytic uremic syndrome (HUS). TTP is characterized by the occurrence of intravasal clotting with formation of microthrombi in very small vessels, which can attack all organs. HUS is an acute syndrome, in which the aggregation of platelets, hemolysis, thrombosis in the microcirculation and consecutive multiorgan failure occurs. The term TMA also comprises secondary forms, which occur, in particular, after infections, taking of medicaments (ciclosporin, mitomycin, metamizole, inter alfa), endocarditis, collagenosis, malignant tumors, transplants and in pregnancy.

Moreover, for example, diabetic microangiopathies (diabetic retinopathy, glomerulopathy, trophic disorders, diabetic gangrene) and also venous occlusive diseases of the liver, cerebral vasculitis, and microthromboses of the placenta, and thus the repeated miscarriages resulting therefrom, can also be treated.

The present invention further relates to the use of selective factor Xa inhibitors for the production of a medicament for the treatment and/or prophylaxis of occlusive syndromes, in particular occlusive syndromes resulting on the skin and other organs, of primary forms of thrombotic microangiopathies (TMA), in particular of thrombotic thrombocytopenic purpura (TTP) and of hemolytic uremic syndrome (HUS), of secondary forms of TMA, in particular after infections, taking of medicaments, endocarditis, collagenosis, malignant tumors, transplants and secondary forms of TMA occurring in pregnancy, of diabetic microangiopathies, in particular diabetic retinopathy, glomerulopathy, trophic disorders and diabetic gangrene, of venous occlusive diseases of the liver, cerebral vasculitis and microthromboses of the placenta, and the repeated miscarriages resulting therefrom.

The present invention further relates to the use of the compounds according to the invention for the production of a medicament for the treatment and/or prophylaxis of occlusive syndromes, in particular occlusive syndromes resulting on the skin and other organs, of primary forms of thrombotic microangiopathies (TMA), in particular thrombotic thrombocytopenic purpura (TTP) and of the hemolytic uremic syndrome (HUS), of secondary forms of TMA, in particular after infections, taking of medicaments, endocarditis, collagenosis, malignant tumors, transplants and secondary forms of TMA occurring in pregnancy, of diabetic microangiopathies, in particular diabetic retinopathy, glomerulopathy, trophic disorders and diabetic gangrene, of venous occlusive diseases of the liver, cerebral vasculitis and microthromboses of the placenta, and the repeated miscarriages resulting therefrom.

In the case of progressive damage, especially in hypoxemic areas such as, for example, in the case of retinopathies, neovascularization (angiogenesis) and thus vitreous body hemorrhages and retinal detachments in the retina occur. The activation of blood clotting induced by tissue thromboplastin (tissue factor; TF) promotes angiogenesis. A number of proteases (factor VIIa, TF-VIIa-Xa complex, factor FXa, thrombin) and their receptors, PAR1, PAR2 (protease-activatable receptors), are involved in this process. By inhibition of factor Xa, the formation of thrombin is inhibited and thus the activation of PAR1, the further generation of VIIa by thrombin and thus in turn the TF-Vila-mediated activation of PAR1 and PAR2, and also the activation of PAR2 by FXa. Therefore FXa inhibitors are also suitable for reducing or preventing the harmful capillary buds resulting in the case of microangiopathies.

The present invention further relates to the use of selective factor Xa inhibitors for the production of a medicament for the treatment and/or prophylaxis of harmful capillary buds resulting in the case of microangiopathies.

The present invention further relates to the use of the compounds according to the invention for the production of a medicament for the treatment and/or prophylaxis of harmful capillary buds resulting in the case of microangiopathies.

The present invention further relates to a procedure for the control of microangiopathies in humans and animals by administration of an efficacious amount of at least one selective factor Xa inhibitor or of a medicament comprising at least one selective factor Xa inhibitor in combination with an inert, non-toxic, pharmaceutically suitable excipient.

The present invention further relates to a procedure for the control of microangiopathies in humans and animals by administration of an efficacious amount of at least one compound according to the invention or of a medicament comprising at least one compound according to the invention in combination with an inert, non-toxic, pharmaceutically suitable excipient.

The present invention further relates to a procedure for the control of harmful capillary buds resulting in the case of microangiopathies in humans and animals by administration of an efficacious amount of at least one selective factor Xa inhibitor or of a medicament comprising at least one selective factor Xa inhibitor in combination with an inert, non-toxic, pharmaceutically suitable excipient.

The present invention further relates to a procedure for the control of harmful capillary buds resulting in the case of microangiopathies in humans and animals by administration of an efficacious amount of at least one compound according to the invention or of a medicament comprising at least one compound according to the invention in combination with an inert, non-toxic, pharmaceutically suitable excipient.

The medicaments to be prepared corresponding to the use according to the invention or to be used according to the invention contain at least one compound according to the invention, customarily together with one or more inert, non-toxic, pharmaceutically suitable excipients.

The compounds according to the invention can act systemically and/or locally. For this purpose, they can be administered in a suitable manner, such as, for example, orally, parenterally, pulmonarily, nasally, sublingually, lingually, buccally, rectally, dermally, transdermally, conjunctivally, otically or as an implant or stent.

For these administration routes, the compounds according to the invention can be administered in suitable administration forms.

For oral administration, suitable administration forms are those functioning according to the prior art, releasing the compounds according to the invention rapidly and/or in modified form, which contain the compounds according to the invention in crystalline and/or amorphized and/or dissolved form, such as, for example, tablets (noncoated or coated tablets, for example having enteric coatings or coatings dissolving with a delay or insoluble coatings, which control the release of the compound according to the invention), tablets disintegrating rapidly in the oral cavity or films/wafers, films/lyophilizates, capsules (for example hard or soft gelatin capsules), coated tablets, granules, pellets, powders, emulsions, suspensions, aerosols or solutions.

Parenteral administration can be carried out circumventing an absorption step (e.g. intravenously, intraarterially, intracardially, intraspinally or intralumbarly) or with the insertion of an absorption (e.g. intramuscularly, subcutaneously, intracutaneously, percutaneously or intraperitoneally). For parenteral administration, suitable administration forms are, inter alia, injection and infusion preparations in the form of solutions, suspensions, emulsions, lyophilizates or sterile powders.

For the other administration routes, for example, inhalation pharmaceutical forms are suitable (inter alia powder inhalers, nebulizers), nose drops, solutions or sprays, tablets to be administered lingually, sublingually or buccally, films/wafers or capsules, suppositories, ear or eye preparations, vaginal capsules, aqueous suspensions (lotions, shake mixtures), lipophilic suspensions, ointments, creams, transdermal therapeutic systems (e.g. patches), milk, pastes, foams, dusting powders, implants or stents.

Oral and parenteral administration are preferred, in particular oral administration.

The compounds according to the invention can be converted into the administration forms mentioned. This can be carried out in a manner known per se by mixing with inert, non-toxic, pharmaceutically suitable excipients. These excipients include, inter alia, vehicles (for example microcrystalline cellulose, lactose, mannitol), solvents (e.g. liquid polyethylene glycols), emulsifiers and dispersants or wetting agents (for example sodium dodecylsulfate, polyoxysorbitan oleate), binders (for example polyvinylpyrrolidone), synthetic and natural polymers (for example albumin), stabilizers (e.g. antioxidants such as, for example, ascorbic acid), colorants (e.g. inorganic pigments such as, for example, iron oxides) and taste and/or odor corrigents.

In general, it has proved advantageous in the case of parenteral administration to administer amounts of approximately 0.001 to 1 mg/kg, preferably approximately 0.01 to 0.5 mg/kg of body weight, to achieve efficacious results. In the case of oral administration, the dose is approximately 0.01 to 100 mg/kg, preferably approximately 0.01 to 20 mg/kg and very particularly preferably 0.1 to 10 mg/kg, of body weight.

In spite of this, it may optionally be necessary to depart from the amounts mentioned, namely depending on body weight, route of administration, individual behavior toward the active substance, type of preparation and time or interval at which administration takes place. Thus, in some cases it may be adequate to manage with less than the abovementioned minimum amount, while in other cases the upper limit mentioned must be exceeded. In the case of the administration of larger amounts, it may be advisable to divide these into a number of individual doses over the course of the day.

The following working examples illustrate the invention. The invention is not restricted to the examples.

The percentages in the following tests and examples are, if not stated otherwise, percentages by weight; parts are parts by weight. Solvent ratios, dilution ratios and concentration data of liquid/liquid solutions relate, if not stated otherwise, to the volume in each case.

### EXAMPLES

### A. Assessment of the Physiological Activity

The compounds of the formula (I) act, in particular, as selective inhibitors of blood clotting factor Xa and do not also inhibit other serine proteases such as plasmin or trypsin or only inhibit them at markedly higher concentrations.

Those inhibitors of blood clotting factor Xa are designated as “selective” in which the IC₅₀ values for the factor Xa inhibition are smaller by at least 100-fold compared to the IC₅₀ values for the inhibition of other serine proteases, in particular plasmin and trypsin, reference being made with respect to the test methods for the selectivity to the test methods of Examples A.a.1) and A.a.2) described below.

Advantageous pharmacological properties of the compounds which can be used according to the invention can be determined by the following methods.

### a) Test Description (In Vitro)

### a.1) Measurement of the Factor Xa Inhibition

The enzymatic activity of human factor Xa (FXa) is measured by means of the conversion of a chromogenic substrate specific for FXa. In this, factor Xa cleaves p-nitroaniline from the chromogenic substrate. The determinations are carried out in microtiter plates as follows.

The test substances are dissolved in DMSO in different concentrations and incubated at 25° C. for 10 minutes with human FXa (0.5 nmol/l dissolved in 50 mmol/l of tris buffer [C,C,C-tris(hydroxymethyl)-aminomethane], 150 mmol/l of NaCl, 0.1% BSA (bovine serum albumin), pH=8.3). Pure DMSO is used as a control. Subsequently, the chromogenic substrate (150 μmol/l of Pefachrome® FXa from Pentapharm) is added. After an incubation period of 20 minutes at 25° C., the extinction at 405 nm is determined. The extinctions of the test batches with test substance are compared with the control batches without test substance and the IC₅₀ values are calculated therefrom.

### a.2) Determination of the Selectivity

For the detection of the selective FXa inhibition, the test substances are investigated for their inhibition of other human serine proteases such as trypsin and plasmin. For the determination of the enzymatic activity of trypsin (500 mU/ml) and plasmin (3.2 nmol/l), these enzymes are dissolved in tris buffer (100 mmol/l, 20 mmol/l CaCl₂, pH=8.0) and incubated with test substance or solvent for 10 minutes. Subsequently, the enzymatic reaction is started by addition of the corresponding specific chromogenic substrates (Chromozym Trypsin® and Chromozym Plasmin®; Roche Diagnostics) and the extinction is determined at 405 nm after 20 minutes. All determinations are carried out at 37° C. The extinctions of the test batches with test substance are compared with the control samples without test substance and the IC₅₀ values are calculated therefrom.

### a.3) Determination of the Anticoagulatory Action

The anticoagulatory action of the test substances is determined in vitro in human and rabbit plasma. For this, blood is taken in a mixing ratio of sodium citrate/blood 1/9 using a 0.11 molar sodium citrate solution as a receiver. The blood is well mixed immediately after taking and centrifuged for 10 minutes at about 2500 g. The supernatant is pipetted off. The prothrombin time (PT, synonyms: thromboplastin time, Quick test) is determined in the presence of varying concentrations of test substance or the corresponding solvent using a commercially available test kit (Neoplastin® from Boehringer Mannheim or Hemoliance® RecombiPlastin, Fa. from Instrumentation Laboratory). The test compounds are incubated with the plasma for 3 minutes at 37° C. Subsequently, clotting is induced by addition of thromboplastin and the time of onset of clotting is determined. The concentration of test substance which causes a doubling of the prothrombin time is determined.

### b) Determination of the Antithrombotic Action (In Vivo)

### b.1) Arteriovenous Shunt Model (Rat)

Fasting male rats (strain: HSD CPB:WU) having a weight of 200-250 g are anesthetized using a Rompun/Ketavet solution (12 mg/kg/50 mg/kg). Thrombus formation is induced in an arteriovenous shunt following the method described by Christopher N. Berry et al., Br. J. Pharmacol. (1994), 113, 1209-1214. For this, the left jugular vein and the right carotid artery are exposed. An extracorporeal shunt is arranged between the two vessels by means of a 10 cm long polyethylene tube (PE 60). This polyethylene tube was linked in the center to a further 3 cm long polyethylene tube (PE 160), which contained a nylon thread which was roughened and linked to a loop for the production of a thrombogenic surface. The extracorporeal circulation is maintained for 15 minutes. The shunt is then removed and the nylon thread with the thrombus is immediately weighed. The unloaded weight of the nylon thread had been determined before the start of the experiment. Before starting the extracorporeal circulation, test substances are administered to conscious animals either intravenously via the caudal vein or orally by means of stomach tube. Results obtained in this way are shown in Table 1:

TABLE 1
Antithrombotic action in the arteriovenous shunt model (rat)
after oral or intravenous dosage

| Example | ED₅₀ [mg/kg] p.o. | ED₅₀ [mg/kg] i.v. |
|---|---|---|
| 1 | | 10 |
| 17 | | 6 |
| 44 | 3 | |
| 95 | | 3 |
| 114 | | 3 |
| 115 | | 3 |
| 123 | 3 | |
| 162 | | 3 |

### b.2) Arteriovenous Shunt Model (Rabbit)

Fasting male ratsrabbits (strain: HSD CPB:WUEsd: NZW) with a weight of 200-250 g are anesthetized by intramuscular dosage with a Rompun/Ketavet solution (12 5 mg/kg or 50 40 mg/kg). Thrombus formation is induced in an arteriovenous shunt following the method described by Christopher C. N. Berry et al., Br. J. Pharmacol [Semin. Thromb. Hemost. (19941996), 11322, 1209-1214 233-241]. For this, the left jugular vein and the right carotid artery are exposed. An extracorporeal shunt is arranged between the two vessels by means of a 10 cm long polyethylene tube (PE 60) venous catheter. This polyethylene tube catheter is linked in the center to a further, 3 4 cm long polyethylene tube (PE 160, Becton Dickenson), which contains a nylon thread which is roughened and arranged to give a loop for the production of a thrombogenic surface. The extracorporeal circulation is maintained for 15 minutes. The shunt is then removed and the nylon thread with the thrombus is immediately weighed. The empty weight of the nylon thread has been determined before the start of the experiment. The test substances are administered to conscious animals before applying the extra-corporeal circulation either intravenously via the caudal vein, an ear vein or orally by means of stomach tube.

### B. Exemplary Embodiments of Pharmaceutical Compositions

The compounds according to the invention can be converted to pharmaceutical preparations in the following way:

### Tablet:

### Composition:

100 mg of the compound according to the invention, 50 mg of lactose (monohydrate), 50 mg of cornstarch (native), 10 mg of polyvinylpyrrolidone (PVP 25) (BASF, Ludwigshafen, Germany) and 2 mg of magnesium stearate.

Tablet weight 212 mg. diameter 8 mm, radius of curvature 12 mm.

### Preparation:

The mixture of compound according to the invention, lactose and starch is granulated with a 5% strength solution (m/m) of the PVP in water. The granules are mixed with the magnesium stearate for 5 minutes after drying. This mixture is compressed using a customary tablet press (for format of the tablet see above). As a standard value for the compression, a compressive force of 15 kN is used.

### Orally Administrable Suspension:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to an individual dose of 100 mg of the compound according to the invention.

### Preparation:

The Rhodigel is suspended in ethanol and the compound according to the invention is added to the suspension. The addition of the water is carried out with stirring. The mixture is stirred for about 6 h up to the completion of the swelling of the Rhodigel.

### Orally Administrable Solution:

### Composition:

500 mg of the compound according to the invention, 2.5 g of polysorbate and 97 g of polyethylene glycol 400.20 g of oral solution correspond to an individual dose of 100 mg of the compound according to the invention.

### Preparation:

The compound according to the invention is suspended in the mixture of polyethylene glycol and polysorbate with stirring. The stirring process is continued up to complete dissolution of the compound according to the invention.

i.v. solution:

The compound according to the invention is dissolved in a concentration below the saturation solubility in a physiologically tolerable solvent (e.g. isotonic saline solution, glucose solution 5% and/or PEG 400 solution 30%). The solution is sterile filtered and filled into sterile and pyrogen-free injection containers.

### C. Preparation Examples

### Starting Compounds

The preparation of 3-morpholinone is described in U.S. Pat. No. 5,349,045.

The preparation of N-(2,3-epoxypropyl)phthalimide is described in J.-W. Chern et al. Tetrahedron Lett. 1998, 39, 8483.

The substituted anilines can be obtained, for example, by reacting 4-fluoronitrobenzene, 2,4-difluoronitrobenzene or 4-chloronitrobenzene with the appropriate amines or amides in the presence of a base. This can also be done using Pd catalysts such as Pd(OAc)₂/DPPF/NaOt-Bu (Tetrahedron Lett. 1999, 40, 2035) or copper (Renger, Synthesis 1985, 856; Aebischer et al., Hetero-cycles 1998, 48, 2225). In the same manner, haloaromatics without a nitro group can first be converted to the corresponding amides, in order subsequently to nitrate them in the 4-position (U.S. Pat. No. 3,279,880).

### I. 4-(4-Morpholin-3-onyl)-nitrobenzene

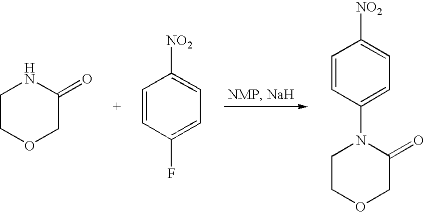

2 mol (202 g) of morpholin-3-one (E. Pfeil, U. Harder, Angew. Chem. 79, 1967, 188) are dissolved in 2 l of N-methylpyrrolidone (NMP). Over a period of 2 h, the addition of 88 g (2.2 mol) of sodium hydride (60% in paraffin) is then carried out in portions. After completion of the evolution of hydrogen, 282 g (2 mol) of 4-fluoronitrobenzene are added dropwise in the course of 1 h with cooling at room temperature and the reaction mixture is stirred overnight. Subsequently, 1.7 l of the volume of liquid are distilled off at 12 mbar and 76° C., the residue is poured onto 2 l of water and this mixture is extracted twice with 1 l each of ethyl acetate. After washing the combined organic phases with water, the mixture is dried over sodium sulfate and the solvent is distilled off in vacuo. Purification is carried out by chromatography on silica gel using hexane/ethyl acetate (1:1) and subsequent crystallization from ethyl acetate. 78 g of the product are obtained as a colorless to brownish solid in 17.6% of theory.

¹H-NMR (300 MHz, CDCl₃): 3.86 (m, 2H, CH₂CH₂), 4.08 (m, 2H, CH₂CH₂), 4.49 (s, 2H, CH₂CO), 7.61 (d, 2H, ³J=8.95 Hz, CHCH), 8.28 (d, 2H, ³J==8.95 Hz, CHCH)

MS (rel. int. %)=222 (74, M⁺), 193 (100), 164 (28), 150 (21), 136 (61), 117 (22), 106 (24), 90 (37), 76 (38), 63 (32), 50 (25)

The following compounds were synthesized analogously:
3-Fluoro-4-(4-morpholin-3-onyl)nitrobenzene
4-(N-Piperidonyl)nitrobenzene
3-Fluoro-4-(N-piperidonyl)nitrobenzene
4-(N-pyrrolidonyl)nitrobenzene
3-Fluoro-4-(N-pyrrolidonyl)nitrobenzene

### II. 4-(4-Morpholin-3-onyl)aniline

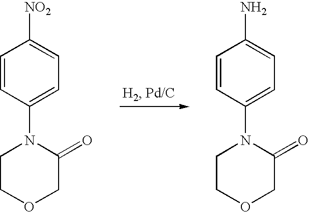

In an autoclave, 63 g (0.275 mol) of 4-(4-morpholin-3-onyl)nitrobenzene are dissolved in 200 ml of tetrahydrofuran, treated with 3.1 g of Pd/C (5% strength) and hydrogenated for 8 h at 70° C. and a hydrogen pressure of 50 bar. After filtration of the catalyst, the solvent is distilled off in vacuo and the product is purified by crystallization from ethyl acetate. 20 g of the product are obtained as a colorless to brownish solid in 37.6% of theory.

Purification can also be carried out by chromatography on silica gel using hexane/ethyl acetate.

¹H-NMR (300 MHz, CDCl₃): 3.67 (m, 2H, CH₂CH₂), 3.99 (m, 2H, CH₂CH₂), 4.27 (s, 2H, CH₂CO), 6.68 (d, 2H, ³J=8.71 Hz, CHCH), 7.03 (d, 2H, ³J=8.71 Hz, CHCH)

MS (rel. int. %)=192 (100, M⁺), 163 (48), 133 (26), 119 (76), 106 (49), 92 (38), 67 (27), 65 (45), 52 (22), 28 (22)

The following compounds were synthesized analogously:
3-Fluoro-4-(4-morpholin-3-onyl)aniline
4-(N-Piperidonyl)aniline
3-Fluoro-4-(N-piperidonyl)aniline
4-(N-Pyrrolidonyl)aniline
3-Fluoro-4-(N-pyrrolidonyl)aniline
   General Method for the Preparation of 4-Substituted Anilines by Reaction of 1-fluoro-4-nitrobenzenes and 1-chloro-4-nitrobenzenes with Primary or Secondary Amines and Subsequent Reduction

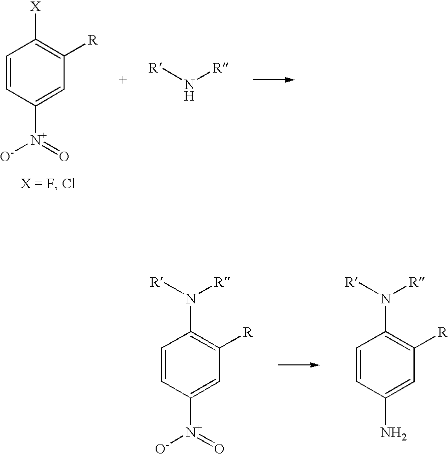

Equimolar amounts of the fluoronitrobenzene or chloronitrobenzene and of the amine are dissolved in dimethyl sulfoxide or acetonitrile (0.1 M to 1 M solution) and stirred overnight at 100° C. After cooling to RT, the reaction mixture is diluted with ether and washed with water. The organic phase is dried over MgSO₄, filtered and concentrated. If a precipitate is formed in the reaction mixture, this is filtered off and washed with ether or acetonitrile. If the product is also to be found in the mother liquor, this is worked up with ether and water as described. The crude products can be purified by chromatography on silica gel (dichloromethane/cyclohexane and dichloromethane/ethanol mixtures).

For the subsequent reduction, the nitro compound is dissolved in methanol, ethanol or ethanol/dichloromethane mixtures (0.01 M to 0.5 M solution), treated with palladium on carbon (10%) and stirred overnight under hydrogen at normal pressure. The mixture is then filtered and concentrated. The crude product can be purified by chromatography on silica gel (dichloromethane/ethanol mixtures) or preparative reversed-phase HPLC (acetonitrile/water mixtures).

Alternatively, iron powder can also be used as a reductant. For this, the nitro compound is dissolved in acetic acid (0.1 M to 0.5 M solution) and six equivalents of iron powder and water (0.3 to 0.5 times volume of acetic acid) are added in portions at 90° C. in the course of 10-15 min. After a further 30 min at 90° C., the mixture is filtered and the filtrate is concentrated. The residue is worked up by extraction with ethyl acetate and 2N sodium hydroxide solution. The organic phase is dried over magnesium sulfate, filtered and concentrated. The crude product can be purified by chromatography on silica gel (dichloromethane/ethanol mixtures) or preparative reversed-phase HPLC (acetonitrile/water mixtures).

The following starting compounds were prepared in an analogous manner:

### III-1. Tert-butyl-1-(4-aminophenyl)-L-prolinate

MS (ESI): m/z (%)=304 (M+H+MeCN, 100), 263 (M+H, 20);

HPLC (method 4): rt=2.79 min.

### III-2. 1-(4-Aminophenyl)-3-piperidinecarboxamide

MS (ESI): m/z (%)=220 (M+H, 100);

HPLC (method 4): rt=0.59 min.

### III-3. 1-(4-Aminophenyl)-4-piperidinecarboxamide

MS (ESI): m/z (%)=220 (M+H, 100);

HPLC (method 4): rt=0.57 min,

### III-4. 1-(4-Aminophenyl)-4-piperidinone

MS (ESI): m/z (%)=191 (M+H, 100);

HPLC (method 4): rt=0.64 min.

### III-5. 1-(4-Aminophenyl)-L-prolinamide

MS (ESI): m/z (%)=206 (M+H, 100);

HPLC (method 4): rt=0.72 min.

### III-6. [1-(4-Aminophenyl)-3-piperidinyl]methanol

MS (ESI): m/z (%)=207 (M+H, 100);

HPLC (method 4): rt=0.60 min.

### III-7. [1-(4-Aminophenyl)-2-piperidinyl]methanol

MS (ESI): m/z (%)=207 (M+H, 100);

HPLC (method 4): rt=0.59 min.

### III-8. Ethyl-1-(4-aminophenyl)-2-piperidinecarboxylate

MS (ESI): m/z (%)=249 (M+H, 35), 175 (100);

HPLC (method 4): rt=2.43 min.

### III-9. [1-(4-Aminophenyl)-2-pyrrolidinyl]methanol

MS (ESI): m/z (%)=193 (M+H, 45);

HPLC (method 4): rt=039 min.

### III-10. 4-(2-Methylhexahydro-5H-pyrrolo[3,4-d]isoxazol-5-yl)phenylamine

starting from 2-methylhexahydro-2H-pyrrolo[3,4-d]isoxazole (Ziegler, Carl B., et al.; J. Heterocycl. Chem.; 25; 2; 1988; 719-723)

MS (EST): m/z (%)=220 (M+H, 50), 171 (100);

HPLC (method 4): rt=0.54 min.

### III-11. 4-(1-Pyrrolidinyl)-3-(trifluoromethyl)aniline

MS (ESI): m/z (%)=231 (M+H, 100);

HPLC (method 7): rt=3.40 min.

### III-12. 3-Chloro-4-(1-pyrrolidinyl)aniline

MS (ESI): m/z (%)=197 (M+H, 100);

HPLC (method 4): rt=0.78 min.

### III.-13. 5-Amino-2-(4-morpholinyl)benzamide

MS (ESI): m/z (%)=222 (M+H, 100);

HPLC (method 4): rt=0.77 min.

### III-14. 3-Methoxy-4-(4-morpholinyl)aniline

MS (ESI): m/z (%)=209 (M+H, 100);

HPLC (method 4): rt=0.67 min.

### III-15. 1-[5-Amino-2-(4-morpholinyl)phenyl]ethanone

MS (ESI): m/z (%)=221 (M+H, 100);

HPLC (method 4): rt=0.77 min.

General Method for the Preparation 4-Substituted Anilines by Reaction of 1-fluoro-4-nitrobenzenes with Amides and Subsequent Reduction

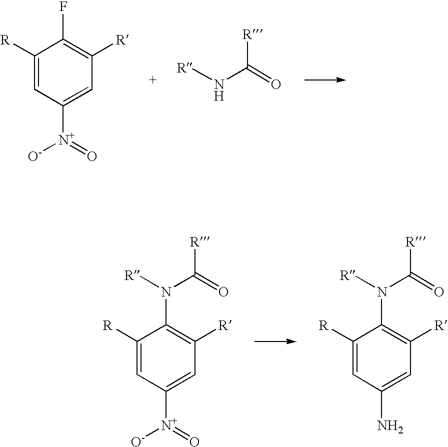

The amide is dissolved in DMF and treated with 1.5 equivalents of potassium tert-butoxide. The mixture is stirred for 1 h at RT, then 1.2 equivalents of 1-fluoro-4-nitrobenzene are added in portions. The reaction mixture is stirred overnight at RT, diluted with ether or ethyl acetate and washed with satd. aq. sodium hydrogencarbonate solution. The organic phase is dried over magnesium sulfate, filtered and concentrated. The crude product can be purified by chromatography on silica gel (dichloromethane/ethanol mixtures).

For the subsequent reduction, the nitro compound is dissolved in ethanol (0.01 M to 0.5 M solution), treated with palladium on carbon (10%) and stirred overnight under hydrogen at normal pressure. The mixture is then filtered and concentrated. The crude product can be purified by chromatography on silica gel (dichloromethane/ethanol mixtures) or preparative reversed-phase HPLC (acetonitrile/water mixtures).

Alternatively, iron powder can also be used as a reductant. For this, the nitro compound is dissolved in acetic acid (0.1 M to 0.5 M solution) and six equivalents of iron powder and water (0.3 to 0.5 times the volume of acetic acid) are added in portions at 90° C. in the course of 10-15 min. After a further 30 min at 90° C., the mixture is filtered and the filtrate is concentrated. The residue is worked up by extraction with ethyl acetate and 2N sodium hydroxide solution. The organic phase is dried over magnesium sulfate, filtered and concentrated. The crude product can be purified by chromatography on silica gel (dichloromethane/ethanol mixtures) or preparative reversed-phase HPLC (acetonitrile/water mixtures).

The following starting compounds were prepared in an analogous manner:

### IV-1. 1-[4-Amino-2-(trifluoromethyl)phenyl]-2-pyrrolidinone

MS (ESI): m/z (%)=245 (M+H, 100);

HPLC (method 4): rt=2.98 min

### IV-2. 4-[4-Amino-2-(trifluoromethyl)phenyl]-3-morpholinone

MS (ESI): m/z (%)=261 (M+H, 100);

HPLC (method 4): rt=2.54 min.

### IV-3. 4-(4-Amino-2-chlorophenyl)-3-morpholinone

MS (ESI): m/z (%)=227 (M+H, 100);

HPLC (method 4): rt=1.96 min.

### IV-4. 4-(4-Amino-2-methylphenyl)-3-morpholinone

MS (ESI): m/z (%)=207 (M+H, 100);

HPLC (method 4): rt=0.71 min.

### IV-5. 5-Amino-2-(3-oxo-4-morpholinyl)benzonitrile

MS (ESI): m/z (%)=218 (M+H, 100);

HPLC (method 4): rt=1.85 min.

### IV-6. 1-(4-Amino-2-chlorophenyl)-2-pyrrolidinone

MS (ESI): m/z (%)=211 (M+H, 100);

HPLC (method 4): rt=2.27 min.

### IV-7. 4-(4-Amino-2,6-dimethylphenyl)-3-morpholinone

starting from 2-fluoro-1,3-dimethyl-5-nitrobenzene (Bartoli et al., J. Org. Chem. 1975, 40, 872):

MS (ESI): m/z (%)=221 (M+H, 100);

HPLC (method 4): rt=0.77 min.

### IV-8. 4-(2,4-Diaminophenyl)-3-morpholinone

starting from 1-fluoro-2,4-dinitrobenzene:

MS (ESI): m/z (%)=208 (M+H, 100);

HPLC (method 4): rt=0.60 min.

### IV-9. 4-(4-Amino-2-chlorophenyl)-2-methyl-3-morpholinone

starting from 2-methyl-3-morpholinone (Pfeil, E.; Harder, U.; Angew. Chem. 1967, 79, 188):

MS (ESI): m/z (%)=241 (M+1-1, 100);

HPLC (method 4): rt=2.27 min.

### IV-10. 4-(4-Amino-2-chlorophenyl)-6-methyl-3-morpholinone

starting from 6-methyl-3-morpholinone (EP 0 350 002):

MS (ESI): m/z (%)=241 (M+H, 100);

HPLC (method 4): rt=2.43 min.

### Synthesis Examples

The following examples 1 to 13, 17 to 19 and 36 to 57 relate to process variant [A].

### Example 1

### Preparation of 5-chloro-N-{[(5S)-3-(3-fluoro-4-morpholinophenyl)-2-oxo-1,3-oxazolidin-5-yl]methyl}-2-thiophenecarboxamide

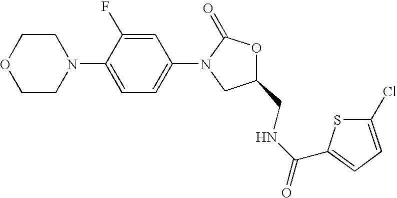

(5S)-5-(Aminomethyl)-3-(3-fluoro-4-morpholinophenyl)-1,3-oxazolidin-2-one (for preparation see S. J. Brickner et al., J. Med. Chem. 1996, 39, 673) (0.45 g, 1.52 mmol), 5-chlorothiophene-2-carboxylic acid (0.25 g, 1.52 mmol) and 1-hydroxy-1H-benzotriazole hydrate (HOBT) (0.3 g, 1.3 equivalents) are dissolved in 9.9 ml of DMF. 0.31 g (1.98 mmol, 1.3 equivalents) of N′-(3-dimethylaminopropyl)-N-ethylcarbodiimide (EDCI) are added and 0.39 g (0.53 ml, 3.05 mmol, 2 equivalents) of diisopropylethylamine (DIEA) are added dropwise at room temperature. The mixture is stirred overnight at room temperature. 2 g of silica gel are added and the batch is evaporated to dryness in vacuo. The residue is chromatographed on silica gel using a toluene-ethyl acetate-gradient. 0.412 g (61.5% of theory) of the target compound is obtained with a melting point (m.p.) of 197° C.

R_{f} (SiO₂, toluene/ethyl acetate 1:1)=0.29 (starting material=0.0);

MS (DCI) 440.2 (M+H), Cl sample;

¹H-NMR (d₆-DMSO, 300 MHz) 2.95 (m, 4H), 3.6 (t, 2H), 3.72 (m, 4H), 3.8 (dd, 1H), 4.12 (t, 1H), 4.75-4.85 (m, 1H), 7.05 (t, 1H), 7.15-7.2 (m, 3H), 7.45 (dd, 1H), 7.68 (d, 1H), 8.95 (t, 1H).

### Example 2

### 5-Chloro-N-{[(5S)-3-(4-morpholinophenyl)-2-oxo-1,3-oxazolidin-5-yl]methyl}-2-thiophenecarboxamide

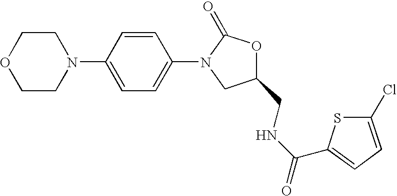

is obtained analogously from benzyl 4-morpholinophenylcarbamate via the stage of (5S)-5-(aminomethyl)-3-(3-fluoro-4-morpholinophenyl)-1,3-oxazolidin-2-one (see Example 1).

M.p.: 198° C.;

IC₅₀ value=43 nM;

R_{f} (SiO₂, toluene/ethyl acetate 1:1)=0.24.

### Example 3

### 5-Chloro-N-({(5S)-3-[3-fluoro-4-(1,4-thiazinan-4-yl)phenyl]-2-oxo-1,3-oxazolidin-5-yl}methyl)-2-thiophenecarboxamide

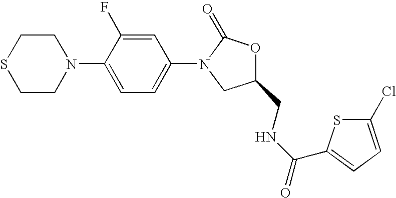

is obtained analogously from (5S)-5-(aminomethyl)-3-[3-fluoro-4-(1,4-thiazinan-4-yl)phenyl]-1,3-oxazolidin-2-one (for preparation see M. R. Barbachyn et al., J. Med. Chem. 1996, 39, 680).

M.p.: 193° C.;

yield: 82%;

R_{f} (SiO₂, toluene/ethyl acetate 1:1)=0.47 (starting material=0.0).

### Example 4

### 5-Bromo-N-({(5S)-3-[3-fluoro-4-(1,4-thiazinan-4-yl)phenyl]-2-oxo-1,3-oxazolidin-5-yl}methyl)-2-thiophenecarboxamide

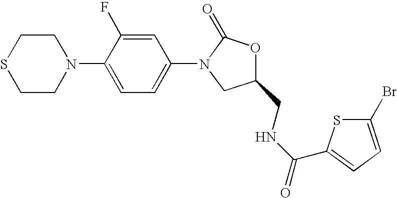

is obtained analogously from 5-bromothiophene-2-carboxylic acid.

M.p.: 200° C.

### Example 5

### N-({(5S)-3-[3-Fluoro-4-(1,4-thiazinan-4-yl)phenyl]-2-oxo-1,3-oxazolidin-5-yl}methyl)-5-methyl-2-thiophenecarboxamide

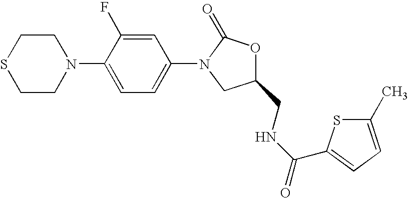

is obtained analogously from 5-methylthiophene-2-carboxylic acid.

M.p.: 167° C.

### Example 6

### 5-Chloro-N-{[(5S)-3-(6-methylthieno[2,3-b]pyridin-2-yl)-2-oxo-1,3-oxazolidin-5-yl]methyl}-2-thiophenecarboxamide

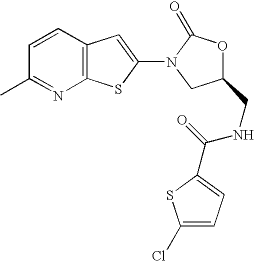

is obtained analogously from (5S)-5-(aminomethyl)-3-(6-methylthieno[2,3-b]pyridin-2-yl)-1,3-oxazolidin-2-one (for preparation see EP 0 785 200).

M.p.: 247° C.

### Example 7

### 5-Chloro-N-{[(5S)-3-(3-methyl-2-oxo-2,3-dihydro-1,3-benzothiazol-6-yl)-2-oxo-1,3-oxazolidin-5-yl]methyl}-2-thiophenecarboxamide

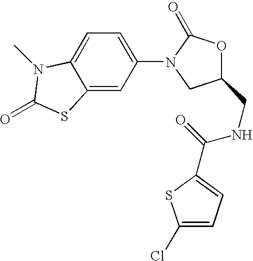

is obtained analogously from 6-[(5S)-5-(aminomethyl)-2-oxo-1,3-oxazolidin-3-yl]-3-methyl-1,3-benzothiazol-2(3H)-one (for preparation see EP 0 738 726).

M.p.: 217° C.

### Example 8

### 5-Chloro-N-[((5S)-3-{3-fluoro-4-[4-(4-pyridinyl)piperazino]phenyl}-2-oxo-1,3-oxazolidin-5-yl)methyl]-2-thiophenecarboxamide

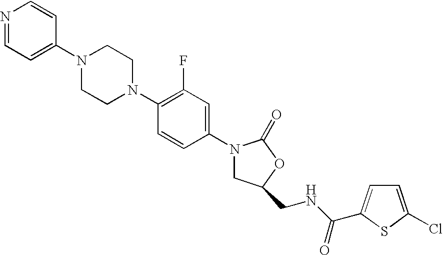

is obtained analogously from (5S)-5-(aminomethyl)-3-{3-fluoro-4-[4-(4-pyridinyl)piperazino]-phenyl}-1,3-oxazolidin-2-one (preparation analogous to J. A. Tucker et al., J. Med. Chem. 1998, 41, 3727).

MS (ESI) 516 (M+H), Cl sample.

### Example 9

### 5-Chloro-N-({(5S)-3-[3-fluoro-4-(4-methylpiperazino)phenyl]-2-oxo-1,3-oxazolidin-5-yl}methyl)-2-thiophenecarboxamide

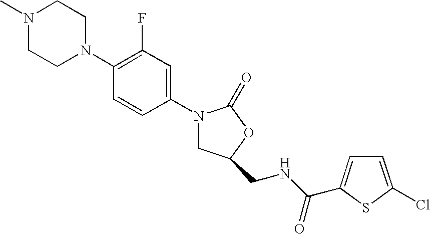

is obtained analogously from (5S)-5-(aminomethyl)-3-[3-fluoro-4-(4-methylpiperazino)phenyl]-1,3-oxazolidin-2-one.

### Example 10

### 5-Chloro-N-({(5S)-3-[3-fluoro-4-(4-tert-butoxycarbonylpiperazin-1-yl)phenyl]-2-oxo-1,3-oxazolidin-5-yl}methyl)-2-thiophenecarboxamide

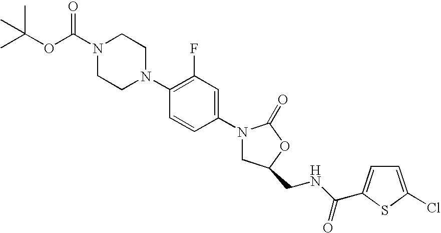

is obtained analogously from (5S)-5-(aminomethyl)-3-[3-fluoro-4-(4-tert-butoxycarbonylpiperazin-1-yl)phenyl]-1,3-oxazolidin-2-one (for preparation see already cited WO 93/23384).

M.p.: 184° C.;

R_{f} (SiO₂, toluene/ethyl acetate 1:1)=0.42.

### Example 11

### 5-Chloro-N-({(5S)-3-[3-fluoro-4-(piperazin-1-yl)phenyl]-2-oxo-1,3-oxazolidin-5-yl}methyl)-2-thiophenecarboxamide

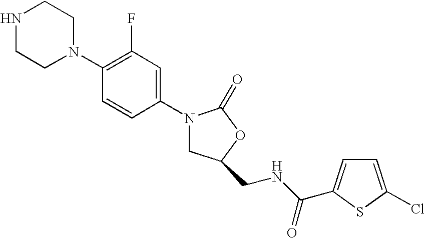

is obtained by reaction of Example 12 with trifluoroacetic acid in methylene chloride.

IC₅₀ value=140 nM;

¹H-NMR [d₆-DMSO]: 3.01-3.25 (m, 8H), 3.5-3.65 (m, 2H), 3.7-3.9 (m, 1H), 4.05-4.2 (m, 1H), 4.75-4.9 (m, 1H), 7.05-7.25 (m, 3H), 7.5 (dd, 1H), 7.7 (d, 1H), 8.4 (broad s, 1H), 9.0 (t, 1H).

### Example 12

### 5-Chloro-N-[((5S)-3-(2,4′-bipyridinyl-5-yl)-2-oxo-1,3-oxazolidin-5-yl)methyl]-2-thiophenecarboxamide

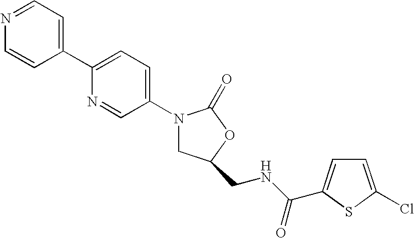

is obtained analogously from (5S)-5-aminomethyl-3-(2,4′-bipyridinyl-5-yl)-2-oxo-1,3-oxazolidin-2-one (for preparation see EP 0 789 026).

R_{f} (SiO₂, ethyl acetate/ethanol 1:2)=0.6;

MS (ESI) 515 (M+H), Cl sample.

### Example 13

### 5-Chloro-N-{[(5S)-2-oxo-3-(4-piperidinophenyl)-1,3-oxazolidin-5-yl]methyl}-2-thiophenecarboxamide

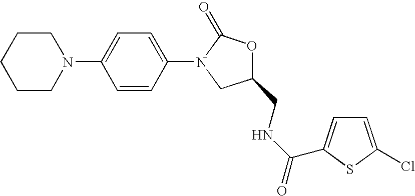

is obtained from 5-(hydroxymethyl)-3-(4-piperidinophenyl)-1,3-oxazolidin-2-one (for preparation see DE 2708236) by mesylation, reaction with potassium phthalimide, hydrazinolysis and reaction with 5-chlorothiophene-2-carboxylic acid.

R_{f} (SiO₂, ethyl acetate/toluene 1:1)=0.31;

m.p. 205° C.

### Example 17

### 5-Chloro-N-({(5S)-2-oxo-3-[4-(2-oxo-1-pyrrolidinyl)phenyl]-1,3-oxazolidin-5-yl}methyl)-2-thiophenecarboxamide

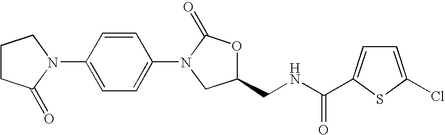

Starting from 1-(4-aminophenyl)pyrrolidin-2-one (for preparation see Reppe et al., Justus Liebigs Ann. Chem.; 596; 1955; 209), in analogy to the known synthesis scheme (see S. J. Brickner et al., J. Med. Chem. 1996, 39, 673), by reaction with benzyloxycarbonyl chloride, subsequent reaction with R-glycidyl butyrate, mesylation, reaction with potassium phthalimide, hydrazinolysis in methanol and reaction with 5-chlorothiophene-2-carboxylic acid, 5-chloro-N-({(5S)-2-oxo-3-[4-(2-oxo-1-pyrrolidinyl)phenyl]-1,3-oxazolidin-5-yl}methyl)-2-thiophenecarboxamide is finally obtained. The 5-chloro-N-({(5S)-2-oxo-3-[4-(2-oxo-1-pyrrolidinyl)phenyl]-1,3-oxazolidin-5-yl}methyl)-2-thiophenecarboxamide obtained in this way has a value of IC₅₀ 4 nM (test method for the IC₅₀ value according to previously described Example A-1. a.1) “Measurement of the factor Xa inhibition”).

M.p.: 229° C.;

R_{f} value (SiO₂, toluene/ethyl acetate 1:1)=0.05 (starting material:=0.0);

MS (ESI): 442.0 (21%, M+Na, Cl sample), 420.0 (72%, M+H, Cl sample), 302.3 (12%), 215(52%), 145 (100%);

¹H-NMR (d₅-DMSO, 300 MHz): 2.05 (m, 2H), 2.45 (m, 2H), 3.6 (t, 2H), 3.77-3.85 (m, 3H), 4.15 (t, 1H), 4.75-4.85 (m, 1H), 7.2 (d, 1H), 7.5 (d, 2H), 7.65 (d, 2H), 7.69 (d, 1H), 8.96 (t, 1H).

The individual stages of the previously described synthesis of Example 17 with the respective precursors are as follows:

4 g (22.7 mmol) of 1-(4-aminophenyl)pyrrolidin-2-one and 3.6 ml (28.4 mmol) of N,N-dimethylaniline are slowly treated at −20° C. with 4.27 g (25.03 mmol) of benzyl chloroformate in 107 ml of tetrahydrofuran. The mixture is stirred for 30 minutes at −20° C. and the whole is subsequently allowed to come to room temperature. 0.5 l ethyl acetate is added and the organic phase is washed with 0.5 l of saturated NaCl solution. The separated organic phase is dried using MgSO₄ and the solvent is evaporated in vacuo. The residue is triturated with diethyl ether and filtered off with suction. 5.2 g (73.8% of theory) of benzyl 4-(2-oxo-1-pyrrolidinyl)phenyl-carbamate are obtained as light beige crystals having a melting point of 174° C.

1.47 g (16.66 mmol) of isoamyl alcohol in 200 ml of tetrahydrofuran under argon are treated dropwise at −10° C. with 7.27 ml of a 2.5 M solution of n-butyllithium (BuLi) in hexane, a further 8 ml of the BuLi solution being necessary up to the change of the added indicator N-benzylidenebenzylamine. The mixture is stirred for 10 minutes at −10° C., cooled to −78° C. and a solution of 4.7 g (15.14 mmol) of benzyl 4-(2-oxo-1-pyrrolidinyl)phenylcarbamate is added slowly. Subsequently, up to the color change of the indicator to pink, another 4 ml of n-BuLi solution are added. The mixture is stirred for 10 minutes at −78° C. and 2.62 g (18.17 mmol) of R-glycidyl butyrate are added and the mixture is stirred again for 30 minutes at −78° C.

The whole is allowed to come to room temperature overnight, 200 ml water are added to the batch and the THF component is evaporated in vacuo. The aqueous residue is extracted with ethyl acetate, and the organic phase is dried using MgSO₄ and evaporated in vacuo. The residue is triturated with 500 ml of diethyl ether and the crystals deposited are filtered off with suction in vacuo.

3.76 g (90% of theory) of (5R)-5-(hydroxymethyl)-3-[4-(2-oxo-1-pyrrolidinyl)phenyl]-1,3-oxazolidin-2-one are obtained with a melting point of 148° C. and an R_{f} value (SiO₂, toluene/ethyl acetate 1:1)=0.04 (starting material=0.3).

3.6 g (13.03 mmol) of (5R)-5-(hydroxymethyl)-3-[4-(2-oxo-1-pyrrolidinyl)phenyl]-1,3-oxazolidin-2-one and 2.9 g (28.67 mmol) of triethylamine are introduced with stirring into 160 ml of dichloromethane at 0° C. 1.79 g (15.64 mmol) of methanesulfonyl chloride are added with stirring and the mixture is stirred for 1.5 hours at 0° C. and for 3 h at room temperature.

The reaction mixture is washed with water and the aqueous phase is extracted again with methylene chloride. The combined organic extracts are dried using MgSO₄ and evaporated. Subsequently, the residue (1.67 g) is dissolved in 70 ml of acetonitrile, treated with 2.62 g (14.16 mmol) of potassium phthalimide and stirred for 45 minutes at 180° C. in a closed vessel in a microwave oven.

The batch is filtered off from insoluble residue, the filtrate is evaporated in vacuo, and the residue (1.9 g) is dissolved in methanol and treated with 0.47 g (9.37 mmol) of hydrazine hydrate. The mixture is boiled for 2 hours, cooled, treated with saturated sodium bicarbonate solution and extracted six times with a total of 2 l of methylene chloride. The combined organic extracts of the crude (5S)-5-(aminomethyl)-3-[4-(2-oxo-1-pyrrolidinyl)phenyl]-1,3-oxazolidin-2-one are dried using MgSO₄ and evaporated in vacuo.

The final stage, 5-chloro-N-({(5S)-2-oxo-3-[4-(2-oxo-1-pyrrolidinyl)phenyl]-1,3-oxazolidin-5-yl}methyl)-2-thiophenecarboxamide, is prepared by dissolving 0.32 g (1.16 mmol) of the (5S)-5-(aminomethyl)-3-[4-(2-oxo-1-pyrrolidinyl)phenyl]-1,3-oxazolidin-2-one prepared above, 5-chlorothiophene-2-carboxylic acid (0.19 g; 1.16 mmol) and 1-hydroxy-1H-benzotriazole hydrate (HOBT) (0.23 g, 1.51 mmol) in 7.6 ml of DMF. 0.29 g (1.51 mmol) of N′-(3-dimethylamino-propyl)-N-ethylcarbodiimide (EDCI) are added and 0.3 g (0.4 ml; 2.32 mmol, 2 equivalents) of diisopropylethylamine (DIEA) are added dropwise at room temperature. The mixture is stirred overnight at room temperature.

The batch is evaporated to dryness in vacuo, and the residue is dissolved in 3 ml of DMSO and chromatographed on an RP-MPLC using an acetonitrile/water/0.5% TFA gradient. The acetonitrile component is evaporated from the appropriate fractions and the precipitated compound is filtered off with suction. 0.19 g (39% of theory) of the target compound is obtained.

The following were prepared in an analogous manner:

### Example 18

### 5-Chloro-N-({(5S)-2-oxo-3-[4-(1-pyrrolidinyl)phenyl]-1,3-oxazolidin-5-yl}methyl)-2-thiophenecarboxamide

Analogously to Example 17, starting from 4-pyrrolidin-1-yl-aniline (Reppe et al., Justus Liebigs Ann. Chem.; 596; 1955; 151) the compound 5-chloro-N-({(5S)-2-oxo-3-[4-(1-pyrrolidinyl)phenyl]-1,3-oxazolidin-5-yl}methyl)-2-thiophenecarboxamide is obtained.

IC₅₀=40 nM;

m.p.: 216° C.;

R_{f} value (SiO₂, toluene/ethyl acetate 1:1)=0.31 [starting material:=0.0].

### Example 19

### 5-Chloro-N-({(5S)-2-oxo-3-[4-(diethylamino)phenyl]-1,3-oxazolidin-5-yl}methyl)-2-thiophenecarboxamide

Analogously, starting from N,N-diethylphenyl-1,4-diamine (U.S. Pat. No. 2,811,555; 1955), the compound 5-chloro-N-({(5S)-2-oxo-3-[4-(diethylamino)phenyl]-1,3-oxazolidin-5-yl}methyl)-2-thiophenecarboxamide is obtained.

IC₅₀=270 nM;

M.p.: 181° C.;

R_{f} value (SiO₂, toluene/ethyl acetate 1:1)=0.25 [starting material:=0.0].

### Example 36

### 5-Chloro-N-({(5S)-3-[2-methyl-4-(4-morpholinyl)phenyl]-2-oxo-1,3-oxazolidin-5-yl}methyl)-2-thiophenecarboxamide

starting from 2-methyl-4-(4-morpholinyl)aniline (J. E. LuValle et al. J. Am. Chem. Soc. 1948, 70, 2223):

MS (ESI): m/z (%)=436 ([M+H]⁺, 100), Cl sample;

HPLC (method 1): rt (%)=3.77 (98).

IC₅₀: 1.26 μM

### Example 37

### 5-Chloro-N-{[(5S)-3-(3-chloro-4-morpholinophenyl)-2-oxo-1,3-oxazolidin-5-yl]methyl}-2-thiophenecarboxamide

starting from 3-chloro-4-(4-morpholinyl)aniline (H. R. Snyder et al. J. Pharm. Sci. 1977, 66, 1204):

MS (ESI): m/z (%)=456 ([M+H]⁺, 100), Cl₂ sample;

HPLC (method 2): rt (%)=4.31 (100).

IC₅₀: 33 nM

### Example 38

### 5-Chloro-N-({(5S)-3-[4-(4-morpholinylsulfonyl)phenyl]-2-oxo-1,3-oxazolidin-5-yl}methyl)-2-thiophenecarboxamide

starting from 4-(4-morpholinylsulfonyl)aniline (Adams et al. J. Am. Chem. Soc. 1939, 61, 2342):

MS (ESI): m/z (%)=486 ([M+H]⁺, 100), Cl sample;

HPLC (method 3): rt (%)=4.07 (100).

IC₅₀: 2 μM

### Example 39

### 5-Chloro-N-({(5S)-3-[4-(1-azetidinylsulfonyl)phenyl]-2-oxo-1,3-oxazolidin-5-yl}methyl)-2-thiophenecarboxamide

starting from 4-(1-azetidinylsulfonyl)aniline:

MS (DCI, NH₃): m/z (%)=473 ([M+NH₄]⁺, 100), Cl sample;

HPLC (method 3): rt (%)=4.10 (100).

IC₅₀: 0.84 μM

### Example 40

### 5-Chloro-N-[((5S)-3-{4-[(dimethylamino)sulfonyl]phenyl}-2-oxo-1,3-oxazolidin-5-yl)methyl]-2-thiophenecarboxamide

starting from 4-amino-N,N-dimethylbenzenesulfonamide (I. K. Khanna et al. J. Med. Chem. 1997, 40, 1619):

MS (ESI): m/z (%)=444 ([M+H]⁺, 100), Cl sample;

HPLC (method 3): rt (%)=4.22 (100).

IC₅₀: 90 nM

General Method for the Acylation of 5-(aminomethyl)-3-[4-(2-oxo-1-pyrrolidinyl)phenyl]-1,3-oxazolidin-2-one with Carbonyl Chlorides.

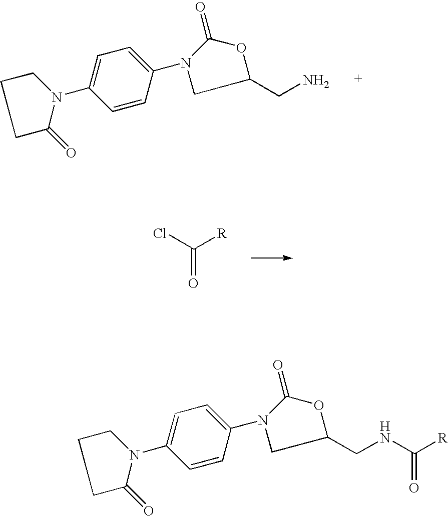

An about 0.1 molar solution of 5-(aminomethyl)-3-[4-(2-oxo-1-pyrrolidinyl)phenyl]-1,3-oxazolidin-2-one (from Example 45) (1.0 eq.) and absolute pyridine (about 6 eq) in absolute dichloromethane is added dropwise under argon at room temperature to the appropriate acid chloride (2.5 eq.). The mixture is stirred for about 4 h at room temperature, before about 5.5 eq of PS-trisamine (Argonaut Technologies) are added. The suspension is gently stirred for 2 h, filtered after diluting with dichloromethane/DMF (3:1) (the resin is washed with dichloromethane/DMF) and the filtrate is concentrated. The product obtained is optionally purified by preparative RP-HPLC.

The following was prepared in an analogous manner:

### Example 41

### N-({2-Oxo-3-[4-(2-oxo-1-pyrrolidinyl)phenyl]-1,3-oxazolidin-5-yl}methyl)-2-thiophenecarboxamide

LC-MS (method 6): m/z (%)=386 (M+H, 100);

LC-MS: rt (%)—-3.04 (100).

IC₅₀: 1.3 μM

General Method for the Preparation of Acyl Derivatives Starting from 5-(aminomethyl)-3-[4-(2-oxo-1-pyrrolidinyl)phenyl]-1,3-oxazolidin-2-one and Carboxylic Acids

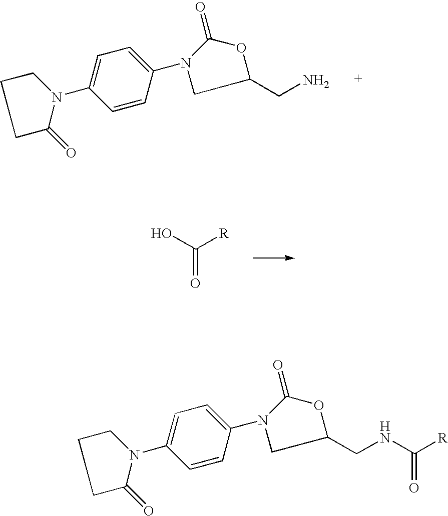

The appropriate carboxylic acid (about 2 eq) and a mixture of absolute dichloromethane/DMF (about 9:1) are added to 2.9 eq. of resin-bound carbodiimide (PS carbodiimide, Argonaut Technologies). After gentle shaking at room temperature for about 15 min, 5-(aminomethyl)-3-[4-(2-oxo-1-pyrrolidinyl)phenyl]-1,3-oxazolidin-2-one (from Example 45) (1.0 eq.) is added and the mixture is shaken overnight, before being filtered off from the resin (washed with dichloromethane), and the filtrate is concentrated. The product obtained is optionally purified by preparative RP-HPLC.

The following were prepared in an analogous manner:

### Example 42

### 5-Methyl-N-({2-oxo-3-[4-(2-oxo-1-pyrrolidinyl)phenyl]-1,3-oxazolidin-5-yl}methyl)-2-thiophenecarboxamide

LC-MS: m/z (%)=400 (M+H, 100);

LC-MS (method 6): rt (%)=3.23 (100).

IC₅₀: 0.16 μM

### Example 43

### 5-Bromo-N-({2-oxo-3-[4-(2-oxo-1-pyrrolidinyl)phenyl]-1,3-oxazolidin-5-yl}methyl)-2-thiophenecarboxamide

LC-MS: m/z (%)=466 (M+H, 100);

LC-MS (method 5): rt (%)=3.48 (78).

IC₅₀: 0.014 μM

### Example 44

### 5-Chloro-N-({(5S)-2-oxo-3-[4-(3-oxo-4-morpholinyl)phenyl]-1,3-oxazolidin-5-yl}methyl)-2-thiophenecarboxamide

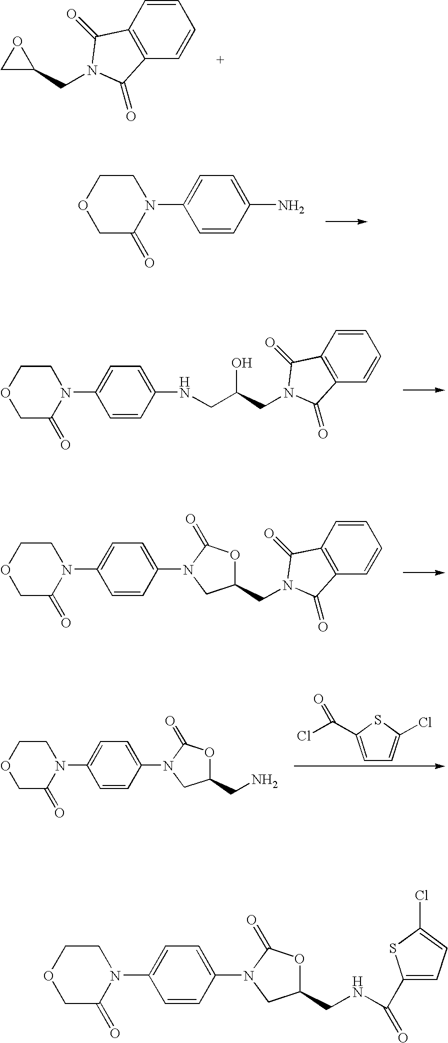

### a) 2-((2R)-2-Hydroxy-3-{[4-(3-oxo-4-morpholinyl)phenyl]amino}propyl)-1H-isoindole-1,3(2H)-dione

A suspension of 2-[(2S)-2-oxiranylmethyl]-1H-isoindole-1,3(2H)-dione (A. Gutcait et al. Tetrahedron Asym. 1996, 7, 1641) (5.68 g, 27.9 mmol) and 4-(4-aminophenyl)-3-morpholinone (5.37 g, 27.9 mmol) in ethanol-water (9:1, 140 ml) is refluxed for 14 h (the precipitate goes into solution, after some time fresh formation of a precipitate). The precipitate (desired product) is filtered off, washed three times with diethyl ether and dried. The combined mother liquors are concentrated in vacuo and, after addition of a second portion of 2-[(2S)-2-oxiranylmethyl]-1H-isoindole-1,3(2H)-dione (2.84 g, 14.0 mmol), suspended in ethanol-water (9:1, 70 ml) and refluxed for 13 h (the precipitate goes into solution, after some time fresh formation of a precipitate). The precipitate (desired product) is filtered off, washed three times with diethyl ether and dried. Total yield: 10.14 g, 92% of theory.

MS (ESI): m/z (%)=418 ([M+Na]⁺, 84), 396 ([M+H]⁺, 93);

HPLC (method 3): rt (%)=3.34 (100).

### b) 2-({(5S)-2-oxo-3-[4-(3-oxo-4-morpholinyl)phenyl]-1,3-oxazolidin-5-yl}methyl)-1H-isoindole-1,3(2H)-dione

N,N′-Carbonyldiimidazole (2.94 g, 18.1 mmol) and dimethylaminopyridine (catalytic amount) are added at room temperature to a suspension of the amino alcohol (3.58 g, 9.05 mmol) in tetrahydrofuran (90 ml) under argon. The reaction suspension is stirred at 60° C. for 12 h (the precipitate goes into solution, after some time fresh formation of a precipitate), treated with a second portion of N,N′-carbonyldiimidazole (2.94 g, 18.1 mmol) and stirred for a further 12 h at 60° C. The precipitate (desired product) is filtered off, washed with tetrahydrofuran and dried. The filtrate is concentrated in vacuo and further product is purified by means of flash chromatography (dichloromethane-methanol mixtures). Total yield: 3.32 g, 87% of theory.

MS (ESI): m/z (%)=422 ([M+H]⁺, 100);

HPLC (method 4): rt (%)=3.37 (100).

### c) 5-Chloro-N-({(5S)-2-oxo-3-[4-(3-oxo-4-morpholinyl)phenyl]-1,3-oxazolidin-5-yl}methyl)-2-thiophenecarboxamide

Methylamine (40% strength in water, 10.2 ml, 0.142 mol) is added dropwise at room temperature to a suspension of the oxazolidinone (4.45 g, 10.6 mmol) in ethanol (102 ml). The reaction mixture is refluxed for 1 h and concentrated in vacuo. The crude product is employed in the next reaction without further purification.

5-Chlorothiophene-2-carbonyl chloride (2.29 g, 12.7 mmol) is added dropwise under argon at 0° C. to a solution of the amine in pyridine (90 ml). The ice cooling is removed and the reaction mixture is stirred for 1 h at room temperature and treated with water. After addition of dichloromethane and phase separation, the aqueous phase is extracted with dichloromethane. The combined organic phases are dried (sodium sulfate), filtered and concentrated in vacuo. The desired product is purified by means of flash chromatography (dichloromethane-methanol mixtures). Total yield: 3.92 g, 86% of theory.

M.p: 232-233° C.;

¹H NMR (DMSO-d⁶, 200 MHz): 9.05-8.90 (t, J=5.8 Hz, 1H), 7.70 (d, J=4.1 Hz, 1H), 7.56 (d, J=9.0 Hz, 2H), 7.41 (d, J=9.0 Hz, 2H), 7.20 (d, J=4.1 Hz, 1H), 4.93-4.75 (m, 1H), 4.27-4.12 (m, 3H), 4.02-3.91 (m, 2H), 3.91-3.79 (dd, J=6.1 Hz, 9.2 Hz, 1H), 3.76-3.66 (m, 2H), 3.66-3.54 (m, 2H);

MS (ESI): m/z (%)=436 [M+H]⁺, 100, Cl sample);

HPLC (method 2): rt (%)=3.60 (100);

[α]²¹_{D}=−38° (c 0.2985, DMSO); ee: 99%.

IC₅₀: 0.7 nM

The following were prepared in an analogous manner:

### Example 45

### 5-Methyl-N-({(5S)-2-oxo-3-[4-(3-oxo-4-morpholinyl)phenyl]-1,3-oxazolidin-5-yl}methyl)-2-thiophenecarboxamide

MS (ESI): m/z (%)=831 ([2M+H]⁺, 100), 416 ([M+H]⁺, 66);

HPLC (method 3): rt (%)=3.65 (100).

IC₅₀: 4.2 nM

### Example 46

### 5-Bromo-N-({(5S)-2-oxo-3-[4-(3-oxo-4-morpholinyl)phenyl]-1,3-oxazolidin-5-yl}methyl)-2-thiophenecarboxamide

MS (ESI): m/z (%)=480 ([M+H]⁺, 100, Br sample);

HPLC (method 3): rt (%)=3.87 (100).

IC₅₀: 0.3 nM

### Example 47

### 5-Chloro-N-{[(5S)-3-(3-isopropyl-2-oxo-2,3-dihydro-1,3-benzoxazol-6-yl)-2-oxo-1,3-oxazolidin-5-yl]methyl}-2-thiophenecarboxamide

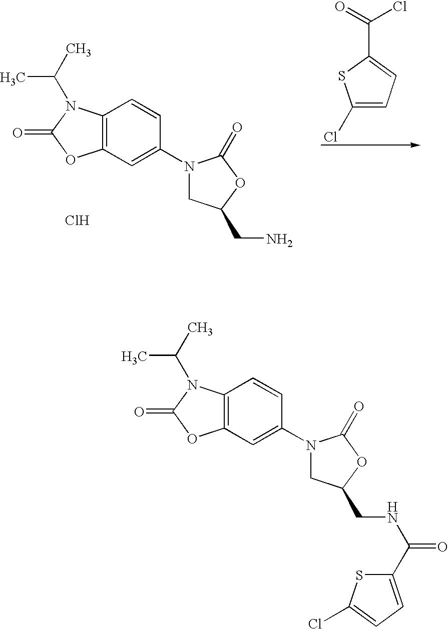

200 mg (0.61 mmol) of 6-[(5S)-5-(aminomethyl)-2-oxo-1,3-oxazolidin-3-yl]-3-isopropyl-1,3-benzoxazol-2(3H)-one hydrochloride (EP 0 738 726) are suspended in 5 ml of tetrahydrofuran and treated with 0.26 ml (1.83 mmol) of triethylamine and 132 mg (0.73 mmol) of 5-chlorothiophene-2-carbonyl chloride. The reaction mixture is stirred overnight at room temperature and subsequently concentrated. The product is isolated by column chromatography (silica gel, methylene chloride/ethanol=50/1 to 20/1). 115 mg (43% of theory) of the desired compound are obtained.

MS (ESI): m/z (%)=436 (M+H, 100);

HPLC (method 4): rt=3.78 min.

The following compounds were prepared in an analogous manner:

| Example | | M.p. | IC₅₀ |
|---|---|---|---|
| No. | Structure | [° C.] | [μm] |
| 48 | 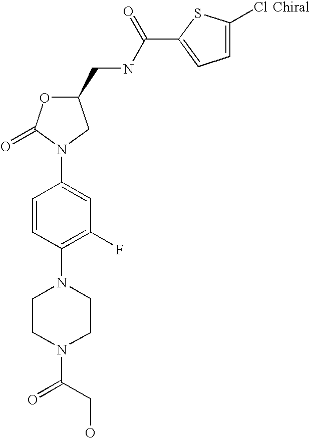 | 210 | 0.12 |
| 49 | 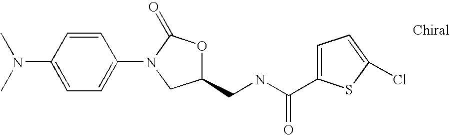 | 234 | 0.074 |
| 50 | 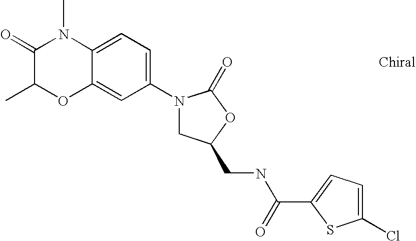 | 195 | 1.15 |
| 51 | 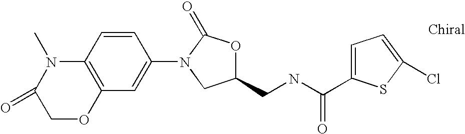 | 212 | 1.19 |
| 52 | 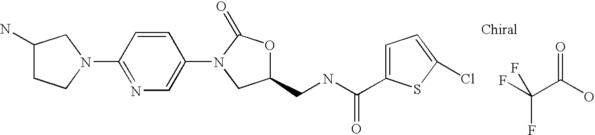 | 160 | 0.19 |
| 53 | 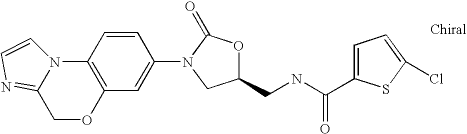 | MS (ESI): m/z (%) = 431 ([M + H]⁺. 100), Cl sample | 0.74 |
| 54 | 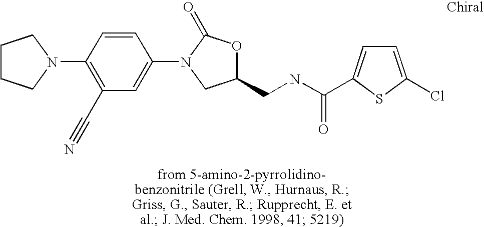 | 221 | 0.13 |
| 55 | 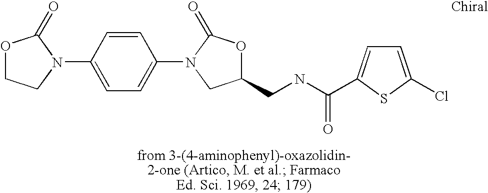 | 256 | 0.04 |
| 56 | 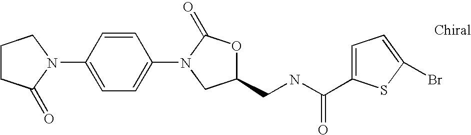 | 218 | 0.004 |
| 57 | 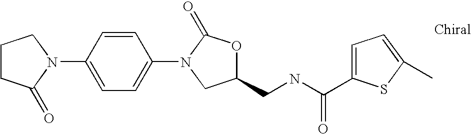 | 226 | 0.58 |
| 255 | 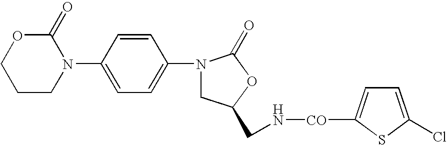 | 228-230 | |

The following Examples 20 to 30 and 58 to 139 relate to process variant [B], Examples 20 and 21 describing the preparation of precursors.

### Example 20

### Preparation of N-allyl-5-chloro-2-thiophenecarboxamide

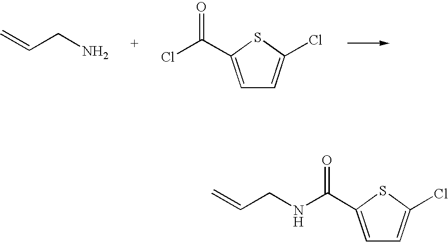

5-Chlorothiophene-2-carbonyl chloride (7.61 g, 42 mmol) is added dropwise to an ice-cooled solution of 2.63 ml (35 mmol) of allylamine in 14.2 ml of absolute pyridine and 14.2 ml of absolute THF. The ice cooling is removed and the mixture is stirred for 3 h at room temperature, before being concentrated in vacuo. The residue is treated with water and the solid is filtered off. The crude product is purified by flash chromatography on silica gel (dichloromethane).

Yield: 7.20 g (99% of theory);

MS (DCI, m/z (%)=219 (M+NH₄, 100), 202 (M+H, 32);

HPLC (method 1): rt (%)=3.96 min (98.9).

### Example 21

### Preparation of 5-chloro-N-(2-oxiranylmethyl)-2-thiophenecarboxamide

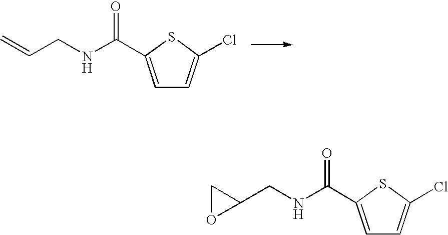

An ice-cooled solution of 2.0 g (9.92 mmol) of N-allyl-5-chloro-2-thiophenecarboxamide in 10 ml of dichloromethane is treated with meta-chloroperbenzoic acid (3.83 g, about 60% strength). The mixture is stirred overnight, warming in the course of this to room temperature, and subsequently washed (three times) with 10% sodium hydrogensulfate solution. The organic phase is washed (twice) with saturated sodium hydrogencarbonate solution and with saturated sodium chloride solution, dried over magnesium sulfate and concentrated. The product is purified by means of chromatography on silica gel (cyclohexane/ethyl acetate 1:1).

Yield: 837 mg (39% of theory);

MS (DCI, NH₄): m/z (%)=253 (M+NH₄, 100), 218 (M+H, 80);

HPLC (method 1): rt (%)=3.69 min (about 80).

General Method for Preparation of Substituted N-(3-amino-2-hydroxypropyl)-5-chloro-2-thiophenecarboxamide Derivatives Starting from 5-chloro-N-(2-oxiranylmethyl)-2-thiophenecarboxamide

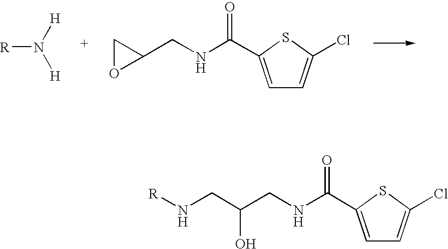

5-Chloro-N-(2-oxiranylmethyl)-2-thiophenecarboxamide (1.0 eq.) is added in portions at room temperature or at temperatures up to 80° C. to a solution of primary amine or aniline derivative (1.5 to 2.5 eq.) in 1,4-dioxane, 1,4-dioxane-water mixtures or ethanol, ethanol-water mixtures (about 0.3 to 1.0 mol/l). The mixture is stirred for 2 to 6 hours, before being concentrated. The product can be isolated from the reaction mixture by chromatography on silica gel (cyclohexane-ethyl acetate mixtures, dichloromethane-methanol mixtures or dichloromethane-methanol-triethylamine mixtures).

The following were prepared in an analogous manner:

### Example 22

### N-[3-(Benzylamino)-2-hydroxypropyl]-5-chloro-2-thiophenecarboxamide

MS (ESI): m/z (%)=325 (M+H, 100);

HPLC (method 1): rt (%)=3.87 min (97.9).

### Example 23

### 5-Chloro-N-[3-(3-cyanoanilino)-2-hydroxypropyl]-2-thiophenecarboxamide

MS (ESI): (%)=336 (M+H, 100);

HPLC (method 2): rt (%)=4.04 min (100).

### Example 24

### 5-Chloro-N-[3-(4-cyanoanilino)-2-hydroxypropyl]-2-thiophenecarboxamide

MS (ESI): m/z (%)=336 (M+H, 100);

HPLC (method 1): rt (%)=4.12 min (100).

### Example 25

### 5-Chloro-N-{3-[4-(cyanomethyl)anilino]-2-hydroxypropyl}-2-thiophenecarboxamide

MS (ESI): m/z (%)=350 (M+H, 100);

HPLC (method 4): rt (%)=3.60 min (95.4).

### Example 26

### 5-Chloro-N-{3-[3-(cyanomethyl)anilino]-2-hydroxypropyl}-2-thiophenecarboxamide

MS (ESI): m/z (%)=350 (M+H, 100);

HPLC (method 4): rt (%)-3.76 min (94.2).

### Example 58

### tert-Butyl 4-[(3-{[(5-chloro-2-thienyl)carbonyl]amino}-2-hydroxypropyl)amino]-benzylcarbamate

Starting from tert-butyl 4-aminobenzylcarbamate (Bioorg. Med. Chem. Lett.; 1997; 1921-1926):

MS (ES-pos): m/z (%)=440 (M+H, 100), (ES-neg): m/z (%)=438 (M−H, 100);

HPLC (method 1): rt (%)=4.08 (100).

### Example 59

### tert-Butyl 4-[(3-{[(5-chloro-2-thienyl)carbonyl]amino}-2-hydroxypropyl)amino]phenylcarbamate

Starting from N-tert-butyloxycarbonyl-1,4-phenylendiamine:

MS (ESI): m/z (%)=426 (M+H, 45), 370 (100);

HPLC (method 1): rt (%)=4.06 (100).

### Example 60

### tert-Butyl 2-hydroxy-3-{[4-(2-oxo-1-pyrrolidinyl)phenyl]amino}propylcarbamate

Starting from 1-(4-aminophenyl)-2-pyrrolidinone (Justus Liebigs Ann. Chem.; 1955; 596; 204):

MS (DCI, NH₃): m/z (%)=350 (M+H, 100);

HPLC (method 1): rt (%)=3.57 (97).

### Example 61

### 5-Chloro-N-(3-{[3-fluoro-4-(3-oxo-4-morpholinyl)phenyl]amino}-2-hydroxypropyl)-2-thiophenecarboxamide

800 mg (3.8 mmol) of 4-(4-amino-2-fluorophenyl)-3-morpholinone and 700 mg (3.22 mmol) of 5-chloro-N-(2-oxiranylmethyl)-2-thiophenecarboxamide are heated under reflux in 15 ml of ethanol and 1 ml of water for 6 hours. The mixture is evaporated in vacuo, filtered off with suction from precipitated crystals after treating with ethyl acetate and, by chromatography of the mother liquor, 276 mg (17% of theory) of the target compound are obtained.

R_{f} (ethyl acetate): 0.25.

### Example 62

### (N-(3-Anilino-2-hydroxypropyl)-5-chloro-2-thiophenecarboxamide

starting from aniline:

MS (DCI, NH₃): m/z (%)=311 ([M+H]⁺, 100), Cl sample;

HPLC (method 3): rt (%)=3.79 (100).

### Example 63

### 5-Chloro-N-(2-hydroxy-3-{[4-(3-oxo-4-morpholinyl)phenyl]amino}propyl)-2-thiophenecarboxamide

starting from 4-(4-aminophenyl)-3-morpholinone:

MS (ESI): m/z (%)=410 ([M+H]⁺, 50), Cl sample;

HPLC (method 3): rt (%)=3.58 (100).

### Example 64

### N-[3-({4-[Acetyl(cyclopropyl)amino]phenyl}amino)-2-hydroxypropyl]-5-chloro-2-thiophenecarboxamide

starting from N-(4-aminophenyl)-N-cyclopropylacetamide:

MS (ESI): m/z (%)=408 ([M+H]⁺, 100), Cl sample;

HPLC (method 3): rt (%)=3.77 (100).

### Example 65

### N-[3-({4-[Acetyl(methyl)amino]phenyl}amino)-2-hydroxypropyl]-5-chloro-2-thiophenecarboxamide

starting from N-(4-aminophenyl)-N-methylacetamide:

MS (EST): m/z (%)=382 (M+H, 100);

HPLC (method 4): rt=3.31 min.

### Example 66

### 5-Chloro-N-(2-hydroxy-3-{[4-(1H-1,2,3-triazol-1-yl)phenyl]amino}propyl)-2-thiophenecarboxamide

starting from 4-(1H-1,2,3-triazol-1-yl)aniline (Bouchet et al.; J. Chem. Soc. Perkin Trans. 2; 1974; 449):

MS (ESI): m/z (%)=378 (M+H, 100);

HPLC (method 4): rt=3.55 min.

### Example 67

### Tert-butyl 1-{4-[(3-{[(5-chloro-2-thienyl)carbonyl]amino}-2-hydroxypropyl)amino]phenyl}-L-prolinate

MS (ESI): m/z (%)=480 (M+H, 100);

HPLC (method 4): rt=3.40 min.

### Example 68

### 1-{4-[(3-{[(5-Chloro-2-thienyl)carbonyl]amino}-2-hydroxypropyl)amino]phenyl}-4-piperidinecarboxamide

MS (ESI): m/z (%)=437 (M+H, 100);

HPLC (method 4): rt=2.39 min.

### Example 69

### 1-{4-[(3-{[(5-Chloro-2-thienyl)carbonyl]amino}-2-hydroxypropyl)amino]phenyl}-3-piperidinecarboxamide

MS (ESI): (%)=437 (M+H, 100);

HPLC (method 4): rt=2.43 min.

### Example 70

### 5-Chloro-N-(2-hydroxy-3-{[4-(4-oxo-1-piperidinyl)phenyl]amino}propyl)-2-thiophenecarboxamide

MS (ESI): m/z (%)=408 (M+H, 100);

HPLC (method 4): rt=2.43 min.

### Example 71

### 1-{4-[(3-{[(5-Chloro-2-thienyl)carbonyl]amino}-2-hydroxypropyl)amino]phenyl}-L-prolinamide

MS (ESI): m/z (%)-423 (M+H, 100);

HPLC (method 4): rt=2.51 min.

### Example 72

### 5-Chloro-N-[2-hydroxy-3-({4-[3-(hydroxymethyl)-1-piperidinyl]phenyl}amino)propyl]-2-thiophenecarboxamide

MS (ESI): m/z (%)=424 (M+H, 100);

HPLC (method 4): rt=2.43 min.

### Example 73

### 5-Chloro-N-[2-hydroxy-3-({4-[2-(hydroxymethyl)-1-piperidinyl]phenyl}amino)propyl]-2-thiophenecarboxamide

MS (ESI): m/z (%)=424 (M+H, 100);

HPLC (method 4): rt=2.49 min.

### Example 74

### Ethyl 1-{4-[3-{[(5-chloro-2-thienyl)carbonyl]amino}-2-hydroxypropyl)amino]phenyl}-2-piperidinecarboxylate

MS (ESI): m/z (%)=466 (M+H, 100);

HPLC (method 4): rt=3.02 min.

### Example 75

### 5-Chloro-N-[2-hydroxy-3-({4-[2-(hydroxymethyl)-1-pyrrolidinyl]phenyl}amino)propyl]-2-thiophenecarboxamide

MS (ESI): m/z (%)=410 (M+H, 100);

HPLC (method 4): rt=2.48 min.

### Example 76

### 5-Chloro-N-(2-hydroxy-3-{[4-(2-methylhexahydro-5H-pyrrolo[3,4-d]isoxazol-5-yl)-phenyl]amino}propyl)-2-thiophenecarboxamide

MS (ESI): m/z (%)=437 (M+H, 100).

HPLC (method 5): rt=1.74 min.

### Example 77

### 5-Chloro-N-(2-hydroxy-3-{[4-(1-pyrrolidinyl)-3-(trifluoromethyl)phenyl]amino}propyl)-2-thiophenecarboxamide

MS (ESI): m/z (%)=448 (M+H, 100);

HPLC (method 4): rt=3.30 min.

### Example 78

### 5-Chloro-N-(2-hydroxy-3-{[4-(2-oxo-1-pyrrolidinyl)-3-(trifluoromethyl)phenyl]-amino}propyl)-2-thiophenecarboxamide

MS (ESI): m/z (%)=462 (M+H, 100);

HPLC (method 4): rt=3.50 min.

### Example 79

### 5-Chloro-N-(3-{[3-chloro-4-(3-oxo-4-morpholinyl)phenyl]amino}-2-hydroxypropyl)-2-thiophenecarboxamide

MS (ESI): m/z (%)=444 (M+H, 100);

HPLC (method 4): rt=3.26 min.

### Example 80

### 5-Chloro-N-(2-hydroxy-3-{[4-(3-oxo-4-morpholinyl)-3-(trifluoromethyl)phenyl]-amino}propyl)-2-thiophenecarboxamide

MS (ESI): m/z (%)=478 (M+H, 100);

HPLC (method 4): rt=3.37 min.

### Example 81

### 5-Chloro-N-(2-hydroxy-3-{[3-methyl-4-(3-oxo-4-morpholinyl)phenyl]amino}propyl)-2-thiophenecarboxamide

MS (ESI): m/z (%)=424 (M+H, 100);

HPLC (method 4): rt=2.86 min.

### Example 82

### 5-Chloro-N-(3-{[3-cyano-4-(3-oxo-4-morpholinyl)phenyl]amino}-2-hydroxypropyl)-2-thiophenecarboxamide

MS (ESI): m/z (%)=435 (M+H, 100);

HPLC (method 4): rt=3.10 min.

### Example 83

### 5-Chloro-N-(3-{[3-chloro-4-(1-pyrrolidinyl)phenyl]amino}-2-hydroxypropyl)-2-thiophenecarboxamide

MS (ESI): m/z (%)=414 (M+H, 100);

HPLC (method 4): rt=2.49 min.

### Example 84

### 5-Chloro-N-(3-{[3-chloro-4-(2-oxo-1-pyrrolidinyl)phenyl]amino}-2-hydroxypropyl)-2-thiophenecarboxamide

MS (ESI): m/z (%)=428 (M+H, 100);

HPLC (method 4): rt=3.39 min.

### Example 85

### 5-Chloro-N-(3-{[3,5-dimethyl-4-(3-oxo-4-morpholinyl)phenyl]amino}-2-hydroxypropyl)-2-thiophenecarboxamide

MS (ESI): m/z (%)=438 (M+H, 100);

HPLC (method 4): rt=2.84 min.

### Example 86

### N-(3-{[3-(Aminocarbonyl)-4-(4-morpholinyl)phenyl]amino}-2-hydroxypropyl)-5-chloro-2-thiophenecarboxamide

MS (ESI): m/z (%)-439 (M+H, 100);

HPLC (method 4): rt=2.32 min.

### Example 87

### 5-Chloro-N-(2-hydroxy-3-{[3-methoxy-4-(4-morpholinyl)phenyl]amino}propyl)-2-thiophenecarboxamide

MS (ESI): m/z (%)—-426 (M+H, 100);

HPLC (method 4): rt=2.32 min.

### Example 88

### N-(3-{[3-Acetyl-4-(4-morpholinyl)phenyl]amino}-2-hydroxypropyl)-5-chloro-2-thiophenecarboxamide

MS (ESI): m/z (%) 438 (M+H, 100);

HPLC (method 4): rt 2.46 min.

### Example 89

### N-(3-{[3-Amino-4-(3-oxo-4-morpholinyl)phenyl]amino}-2-hydroxypropyl)-5-chloro-2-thiophenecarboxamide

MS (ESI): m/z (%)=425 (M+H, 100);

HPLC (method 4): rt 2.45 min.

### Example 90

### 5-Chloro-N-(3-{[3-chloro-4-(2-methyl-3-oxo-4-morpholinyl)phenyl]amino}-2-hydroxypropyl)-2-thiophenecarboxamide

MS (ESI): m/z (%)=458 (M+H, 100);

HPLC (method 4): rt=3.44 min.

### Example 91

### 5-Chloro-N-(3-{[3-chloro-4-(2-methyl-5-oxo-4-morpholinyl)phenyl]amino}-2-hydroxypropyl)-2-thiophenecarboxamide

MS (ESI): m/z (%)=458 (M+H, 100);

HPLC (method 4): rt=3.48 min.

### Example 91a

### 5-Chloro-N-[2-hydroxy-3-({4-[(3-oxo-4-morpholinyl)methyl]phenyl}amino)propyl]-2-thiophenecarboxamide

Starting from 4-(4-aminobenzyl)-3-morpholinone (Surrey et al.; J. Amer. Chem. Soc.; 77; 1955; 633):

MS (ESI): m/z (%)=424 (M+H, 100);

HPLC (method 4): rt 2.66 min.

General Method for Preparation of 3-Substituted 5-chloro-N-[(2-oxo-1,3-oxazolidin-5-yl)methyl]-2-thiophenecarboxamide Derivatives Starting from Substituted N-(3-amino-2-hydroxypropyl)-5-chloro-2-thiophenecarboxamide Derivatives

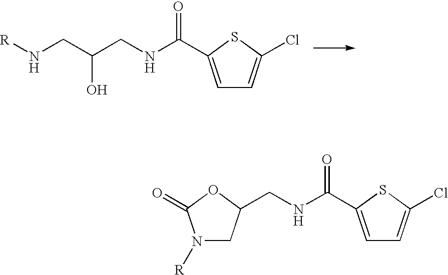

Carbodiimidazole (1.2 to 1.8 eq.) or a comparable phosgene equivalent is added at room temperature to a solution of substituted N-(3-amino-2-hydroxypropyl)-5-chloro-2-thiophenecarboxamide derivative (1.0 eq.) in absolute THF (about 0.1 mol/l). The mixture is stirred at room temperature or optionally at elevated temperature (up to 70° C.) for 2 to 18 h, before being concentrated in vacuo. The product can be purified by chromatography on silica gel (dichloromethane-methanol mixtures or cyclohexane-ethyl acetate mixtures).

The following were prepared in an analogous manner:

### Example 27

### N-[(3-Benzyl-2-oxo-1,3-oxazolidin-5-yl)methyl]-5-chloro-2-thiophenecarboxamide

MS (DCI, NH₄): m/z (%)=372 (M+Na, 100), 351 (M+H, 45);

HPLC (method 1): rt (%)=4.33 min (100).

### Example 28

### 5-Chloro-N-{[3-(3-cyanophenyl)-2-oxo-1,3-oxazolidin-5-yl]methyl}-2-thiophenecarboxamide

MS (DCI, m/z (%)=362 (M+H, 42), 145 (100);

HPLC (method 2): rt (%)=4.13 min (100).

### Example 29

### 5-Chloro-N-({3-[4-(cyanomethyl)phenyl]-2-oxo-1,3-oxazolidin-5-yl}methyl)-2-thiophenecarboxamide

MS (ESI): m/z (%)=376 (M+H, 100);

HPLC (method 4): rt=4.12 min

### Example 30

### 5-Chloro-N-({3-[3-(cyanomethyl)phenyl]-2-oxo-1,3-oxazolidin-5-yl}methyl)-2-thiophenecarboxamide

MS (ESI): m/z (%)=376 (M+H, 100);

HPLC (method 4): rt=4.17 min

### Example 92

### tert-Butyl 4-[5-({[(5-chloro-2-thienyl)carbonyl]amino}methyl)-2-oxo-1,3-oxazolidin-3-yl]benzylcarbamate

starting from Example 58:

MS (ESI): m/z (%)=488 (M+Na, 23), 349 (100);

HPLC (method 1): rt (%)=4.51 (98.5).

### Example 93

### tert-Butyl 4-[5-({[(5-chloro-2-thienyl)carbonyl]amino}methyl)-2-oxo-1,3-oxazolidin-3-yl]phenylcarbamate

starting from Example 59:

MS (ESI): m/z (%)=493 (M+Na, 70), 452 (M+H, 10), 395 (100);

HPLC (method 1): rt (%)=4.41 (100).

### Example 94

### tert-Butyl 2-oxo-3-[4-(2-oxo-1-pyrrolidinyl)phenyl]-1,3-oxazolidin-5-yl}methylcarbamate

starting from Example 60:

MS (DCI, NH₃): m/z (%)=393 (M+NH₄, 100);

HPLC (method 3): rt (%)=3.97 (100).

### Example 95

### 5-Chloro-N-({3-[3-fluoro-4-(3-oxo-4-morpholinyl)phenyl]-2-oxo-1,3-oxazolidin-5-yl}methyl)-2-thiophenecarboxamide

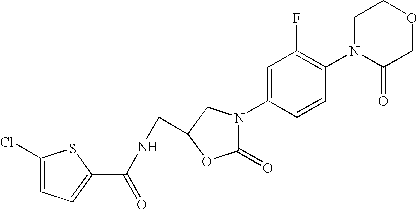

260 mg (0.608 mmol) of 5-chloro-N-(3-{[3-fluoro-4-(3-oxo-4-morpholinyl)phenyl]amino}-2-hydroxypropyl)-2-thiophenecarboxamide (from Example 61), 197 mg (1.22 mmol) of carbonylimidazole and 7 mg of dimethylaminopyridine are refluxed in 20 ml of dioxane for 5 hours. Subsequently, 20 ml of acetonitrile are added and the mixture is stirred in a closed container in a microwave oven for 30 minutes at 180° C. The solution is concentrated in a rotary evaporator and chromatographed on an RP-HPLC column. 53 mg (19% of theory) of the target compound are obtained.

NMR (300 MHz, d₆-DMSO): δ=3.6-3.7 (m, 4H), 3.85 (dd, 1H), 3.95 (m, 2H), 4.2 (m, 1H), 4.21 (s, 2H), 4.85 (m, 1H), 4.18 (s, 2H), 7.19 (d, 1H, thiophene), 7.35 (dd, 1H), 7.45 (t, 1H), 7.55 (dd, 1H), 7.67 (d, 1H, thiophene), 8.95 (t, 1H, CONH).

### Example 96

### 5-Chloro-N-[(2-oxo-3-phenyl-1,3-oxazolidin-5-yl)methyl]-2-thiophenecarboxamide

starting from Example 62:

MS (ESI): m/z (%)=359 ([M+Na]⁺, 71), 337 ([M+H]⁺, 100), Cl sample;

HPLC (method 3): rt (%)=4.39 (100).

IC₅₀: 2 μM

### Example 97

### 5-Chloro-N-({2-oxo-3-[4-(3-oxo-4-morpholinyl)phenyl]-1,3-oxazolidin-5-yl}methyl)-2-thiophenecarboxamide

starting from Example 63:

MS (ESI): m/z (%)=458 ([M+Na]⁺, 66), 436 ([M+H]⁺, 100), Cl sample;

HPLC (method 3): rt (%)=3.89 (100).

IC₅₀: 1.4 nM

### Example 98

### N-[(3-{4-[Acetyl(cyclopropyl)amino]phenyl}-2-oxo-1,3-oxazolidin-5-yl)methyl]-5-chloro-2-thiophenecarboxamide

starting from Example 64:

MS (ESI): m/z (%)=456 ([M+Na]⁺, 55), 434 ([M+H]⁺, 100), Cl sample;

HPLC (method 3): rt (%)=4.05 (100).

IC₅₀: 50 nM

### Example 99

### N-[(3-{4-[Acetyl(methyl)amino]phenyl}-2-oxo-1,3-oxazolidin-5-yl)methyl]-5-chloro-2-thiophenecarboxamide

MS (ESI): m/z (%)=408 (M+H, 30), 449 (M+H+MeCN, 100);

HPLC (method 4): rt=3.66 min.

### Example 100

### 5-Chloro-N-({2-oxo-3-[4-(1H-1,2,3-triazol-1-yl)phenyl]-1,3-oxazolidin-5-yl}methyl)-2-thiophenecarboxamide

MS (ESI): m/z (%)=404 (M+H, 45), 445 (M+H+MeCN, 100);

HPLC (method 4): rt=3.77 min.

### Example 101

### Tert-butyl 1-{4-[5-({[(5-chloro-2-thienyl)carbonyl]amino}methyl)-2-oxo-1,3-oxazolidin-3-yl]phenyl}-L-prolinate

MS (ESI): m/z (%)=450 (M+H−56, 25), 506 (M+H, 100);

HPLC (method 4): rt=5.13 min.

### Example 102

### 1-{4-[5-({[(5-Chloro-2-thienyl)carbonyl]amino}methyl)-2-oxo-1,3-oxazolidin-3-yl]phenyl}-4-piperidinecarboxamide

MS (ESI): m/z (%)=463 (M+H, 100);

HPLC (method 4): rt=2.51 min.

### Example 103

### 1-{4-[5-({[(5-Chloro-2-thienyl)carbonyl]amino}methyl)-2-oxo-1,3-oxazolidin-3-yl]phenyl}-3-piperidinecarboxamide

MS (ESI): m/z (%)=463 (M+H, 100);

HPLC (method 4): rt 2.67 min.

### Example 104

### 5-Chloro-N-({2-oxo-3-[4-(4-oxo-1-piperidinyl)phenyl]-1,3-oxazolidin-5-yl}methyl)-2-thiophenecarboxamide

MS (ESI): m/z (%)=434 (M+H, 40), 452 (M+H+H₂O, 100), 475 (M+H+MeCN, 60);

HPLC (method 4): rt=3.44 min.

### Example 105

### 1-{4-[5-({[(5-Chloro-2-thienyl)carbonyl]amino}methyl)-2-oxo-1,3-oxazolidin-3-yl]phenyl}-L-prolinamide

MS (ESI): m/z (%)=449 (M+H, 100);

HPLC (method 4): rt=3.54 min.

### Example 106

### 5-Chloro-N-[(3-{4-[3-(hydroxymethyl)-1-piperidinyl]phenyl}-2-oxo-1,3-oxazolidin-5-yl)methyl]-2-thiophenecarboxamide

MS (ESI): m/z (%)=450 (M+H, 100);

HPLC (method 5): rt=2.53 min.

### Example 107

### 5-Chloro-N-[(3-{4-[2-(hydroxymethyl)-1-piperidinyl]phenyl}-2-oxo-1,3-oxazolidin-5-yl)methyl]-2-thiophenecarboxamide

MS (ESI): m/z (%)-450 (M+H, 100);

HPLC (method 5): rt=2.32 min.

### Example 108

### Ethyl 1-{-4-[5-({[(5-chloro-2-thienyl)carbonyl]amino}methyl)-2-oxo-1,3-oxazolidin-3-yl]phenyl}-2-piperidinecarboxylate

MS (ESI): m/z (%)=492 (M+H, 100);

HPLC (method 5): rt=4.35 min.

### Example 109

### 5-Chloro-N-[(3-{4-[2-(hydroxymethyl)-1-pyrrolidinyl]phenyl}-2-oxo-1,3-oxazolidin-5-yl)methyl]-2-thiophenecarboxamide

MS (ESI): m/z (%)=436 (M+H, 100);

HPLC (method 4): rt=2.98 min.

### Example 110

### 5-Chloro-N-({2-oxo-3-[4-(1-pyrrolidinyl)-3-(trifluoromethyl)phenyl]-1,3-oxazolidin-5-yl}methyl)-2-thiophenecarboxamide

MS (EST): m/z (%)=474 (M+H, 100);

HPLC (method 4): rt=4.63 min.

### Example 111

### 5-Chloro-N-({3-[4-(2-methylhexahydro-5H-pyrrolo[3,4-d]isoxazol-5-yl)phenyl]-2-oxo-1,3-oxazolidin-5-yl}methyl)-2-thiophenecarboxamide

MS (ESI): m/z (%)=463 (M+H, 100);

HPLC (method 4): rt=2.56 min.

### Example 112

### 5-Chloro-N-({2-oxo-3-[4-(2-oxo-1-pyrrolidinyl)-3-(trifluoromethyl)phenyl]-1,3-oxazolidin-5-yl}methyl)-2-thiophenecarboxamide

MS (EST): m/z (%)=488 (M+H, 100);

HPLC (method 4): rt=3.64 min.

### Example 113

### 5-Chloro-N-({3-[3-chloro-4-(3-oxo-4-morpholinyl)phenyl]-2-oxo-1,3-oxazolidin-5-yl}methyl)-2-thiophenecarboxamide

MS (ESI): m/z (%)=470 (M+H, 100);

HPLC (method 4): rt=3.41 min.

### Example 114

### 5-Chloro-N-({2-oxo-3-[4-(3-oxo-4-morpholinyl)-3-(trifluoromethyl)phenyl]-1,3-oxazolidin-5-yl}methyl)-2-thiophenecarboxamide

MS (ESI): m/z (%)=504 (M+H, 100);

HPLC (method 4): rt=3.55 min.

### Example 115

### 5-Chloro-N-({3-[3-methyl-4-(3-oxo-4-morpholinyl)phenyl]-2-oxo-1,3-oxazolidin-5-yl}methyl)-2-thiophenecarboxamide

MS (ESI): m/z (%)=450 (M+H, 100);

HPLC (method 4): rt=3.23 min.

### Example 116

### 5-Chloro-N-({3-[3-cyano-4-(3-oxo-4-morpholinyl)phenyl]-2-oxo-1,3-oxazolidin-5-yl}methyl)-2-thiophenecarboxamide

MS (ESI): m/z (%)=461 (M+H, 100);

HPLC (method 4): rt=3.27 min.

### Example 117

### 5-Chloro-N-({3-[3-chloro-4-(1-pyrrolidinyl)phenyl]-2-oxo-1,3-oxazolidin-5-yl}methyl)-2-thiophenecarboxamide

MS (ESI): m/z (%)=440 (M+H, 100);

HPLC (method 4): rt=3.72 min.

### Example 118

### 5-Chloro-N-({3-[3-chloro-4-(2-oxo-1-pyrrolidinyl)phenyl]-2-oxo-1,3-oxazolidin-5-yl}methyl)-2-thiophenecarboxamide

MS (ESI): m/z (%)=454 (M+H, 100);

HPLC (method 4): rt=3.49 min.

### Example 119

### 5-Chloro-N-({3-[3,5-dimethyl-4-(3-oxo-4-morpholinyl)phenyl]-2-oxo-1,3-oxazolidin-5-yl}methyl)-2-thiophenecarboxamide

MS (ESI): m/z (%)=464 (M+H, 100);

HPLC (method 4): rt=3.39 min.

### Example 120

### N-({3-[3-(Aminocarbonyl)-4-(4-morpholinyl)phenyl]-2-oxo-1,3-oxazolidin-5-yl}methyl)-5-chloro-2-thiophenecarboxamide

MS (ESI): m/z (%)=465 (M+H, 100);

HPLC (method 4): rt=3.07 min.

### Example 121

### 5-Chloro-N-({3-[3-methoxy-4-(4-morpholinyl)phenyl]-2-oxo-1,3-oxazolidin-5-yl}methyl)-2-thiophenecarboxamide

MS (ESI): m/z (%) 452 (M+H, 100);

HPLC (method 4): rt=2.86 min.

### Example 122

### N-({3-[3-Acetyl-4-(4-morpholinyl)phenyl]-2-oxo-1,3-oxazolidin-5-yl}methyl)-5-chloro-2-thiophenecarboxamide

MS (ESI): m/z (%)=464 (M+H, 100);

HPLC (method 4): rt=3.52 min.

### Example 123

### N-({3-[3-Amino-4-(3-oxo-4-morpholinyl)phenyl]-2-oxo-1,3-oxazolidin-5-yl}methyl)-5-chloro-2-thiophenecarboxamide

MS (ESI): m/z (%)=451 (M+H, 100);

HPLC (method 6): rt 3.16 min.

### Example 124

### 5-Chloro-N-({3-[3-chloro-4-(2-methyl-3-oxo-4-morpholinyl)phenyl]-2-oxo-1,3-oxazolidin-5-yl}methyl)-2-thiophenecarboxamide

MS (ESI): m/z (%)=484 (M+H, 100);

HPLC (method 4): rt=3.59 min.

### Example 125

### 5-Chloro-N-({3-[3-chloro-4-(2-methyl-5-oxo-4-morpholinyl)phenyl]-2-oxo-1,3-oxazolidin-5-yl}methyl)-2-thiophenecarboxamide

MS (ESI): m/z (%)=484 (M+H, 100);

HPLC (method 4): rt=3.63 min.

### Example 125a

### 5-Chloro-N-[(2-oxo-3-{4-[(3-oxo-4-morpholinyl)methyl]phenyl}-1,3-oxazolidin-5-yl)methyl]-2-thiophenecarboxamide

MS (ESI): m/z (%)=450 (M+H, 100);

HPLC (method 4): rt=3.25 min,

The following compounds were moreover prepared via the route of the epoxide opening using an amine and subsequent cyclization to give the corresponding oxazolidinone:

| Example | | M.p. | IC₅₀ |
|---|---|---|---|
| No. | Structure | [° C.] | [μm] |
| 126 | 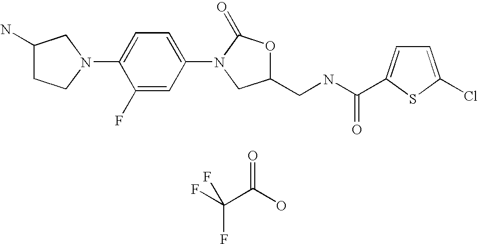 | 229Z | 0.013 |
| 127 | 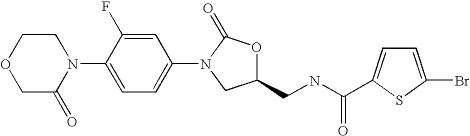 | 159 | 0.0007 |
| 128 | 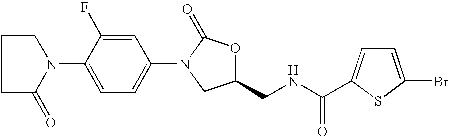 | 198 | 0.002 |
| 129 | 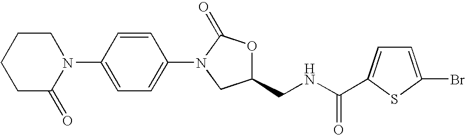 | 196 | 0.001 |
| 130 | 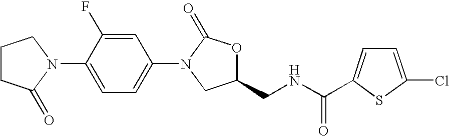 | 206 | 0.0033 |
| 130a | 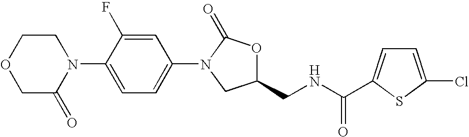 | 194 | |
| 131 | 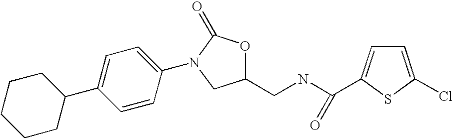 | 195 | 0.85 |
| 132 | 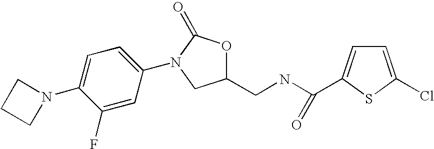 | 206 | 0.12 |
| 133 | 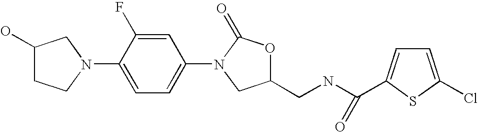 | 217 | 0.062 |
| 134 | 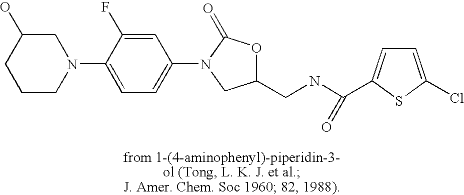 | 207 | 0.48 |
| 135 | 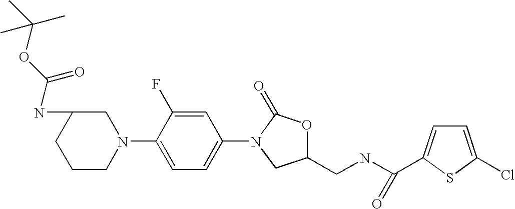 | 202 | 1.1 |
| 136 | 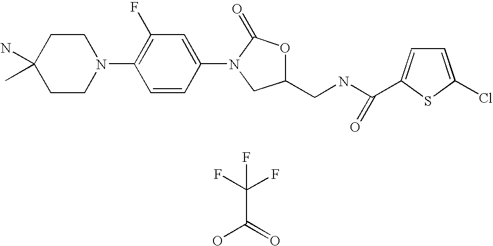 | 239 | 1.2 |
| 137 | 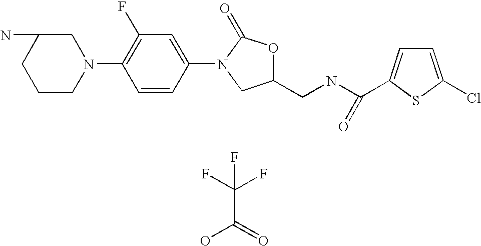 | 219 | 0.044 |
| 138 | 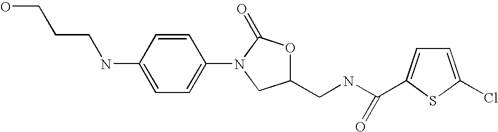 | 95 | 0.42 |
| 139 | 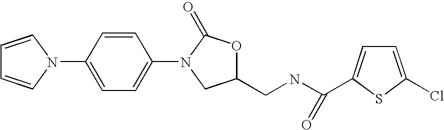 | 217 | 1.7 |

The following Examples 14 to 16 are working examples for the voluntary, i.e. optionally taking place, oxidation process step.

### Example 14

### 5-Chloro-N-({(5S)-3-[3-fluoro-4-(1-oxo-1[lambda]⁴,4-thiazinan-4-yl)phenyl]-2-oxo-1,3-oxazolidin-5-yl}methyl)-2-thiophenecarboxamide

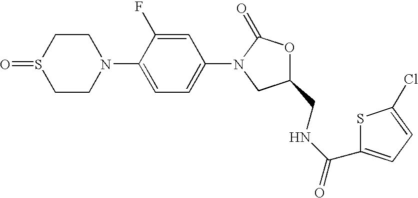

5-Chloro-N-({(5S)-3-[3-fluoro-4-(1,4-thiazinan-4-yl)phenyl]-2-oxo-1,3-oxazolidin-5-yl}methyl)-2-thiophenecarboxamide (0.1 g, 0.22 mmol) from Example 3 in methanol (0.77 ml) is added at 0° C. to a solution of sodium periodate (0.05 g, 0.23 mmol) in water (0.54 ml) and stirred for 3 h at 0° C. Subsequently, 1 ml of DMF is added and the mixture is stirred for 8 h at RT. After addition of a further 50 mg of sodium periodate, the mixture is again stirred overnight at RT. The batch is subsequently treated with 50 ml of water and the insoluble product is filtered off with suction. After washing with water and drying, 60 mg (58% of theory) of crystals are obtained.

M.p.: 257° C.;

R_{f} (silica gel, toluene/ethyl acetate 1:1)=0.54 (starting material=0.46);

IC₅₀ value 1.1 μM;

MS (DCI) 489 (M+NH₄), Cl sample.

### Example 15

### Preparation of 5-chloro-N-({(5S)-3-[4-(1,1-dioxo-1[lambda]⁶,4-thiazinan-4-yl)-3-fluorophenyl]-2-oxo-1,3-oxazolidin-5-yl}methyl)-2-thiophenecarboxamide

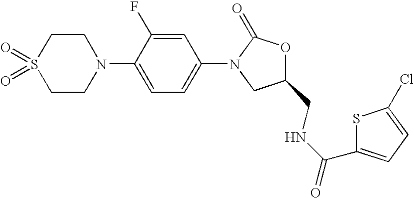

5-Chloro-N-({(5S)-3-[3-fluoro-4-(1,4-thiazinan-4-yl)phenyl]-2-oxo-1,3-oxazolidin-5-yl}methyl)-2-thiophenecarboxamide from Example 3 (0.1 g, 0.22 mmol) in 3.32 ml of a mixture of 1 part of water and 3 parts of acetone is treated with 80 mg (0.66 mmol) of N-methylmorpholine N-oxide (NMO) and 0.1 ml of a 2.5% strength solution of osmium tetroxide in 2-methyl-2-propanol. The mixture is stirred overnight at room temperature and 40 mg of NMO are added again. After a further night, the batch is added to 50 ml of water and extracted three times with ethyl acetate. From the organic phase, after drying and evaporating, 23 mg, and from the aqueous phase after filtering off the insoluble solid with suction, 19 mg (altogether 39% of theory) of the target compound are obtained.

M.p.: 238° C.;

R_{f} (toluene/ethyl acetate 1:1)=0.14 (starting material=0.46);

IC₅₀ value=210 nM;

MS (DCI): 505 (M+NH₄), Cl sample.

### Example 16

### 5-Chloro-N-{[(5S)-3-(3-fluoro-4-morpholinophenyl)-2-oxo-1,3-oxazolidin-5-yl]methyl}-2-thiophenecarboxamide N-oxide

is obtained by treating 5-chloro-N-{[(5S)-3-(3-fluoro-4-morpholinophenyl)-2-oxo-1,3-oxazolidin-5-yl]methyl}-2-thiophenecarboxamide from Example 1 with monoperoxyphthalate acid magnesium salt.

MS (ESI): 456 (M+H, 21%, Cl sample), 439 (100%).

The following examples 31 to 35 and 140 to 147 relate to the voluntary, i.e. optionally taking place, amidination process step.

General method for the preparation of amidines and amidine derivatives starting from cyanomethylphenyl-substituted 5-chloro-N-[(2-oxo-1,3-oxazolidin-5-yl)methyl]-2-thiophenecarboxamide derivatives

The respective cyanomethylphenyl-substituted 5-chloro-N-[(2-oxo-1,3-oxazolidin-5-yl)methyl]-2-thiophenecarboxamide derivative (1.0 eq.) is stirred in a saturated solution of hydrogen sulfide in pyridine (about 0.05-0.1 mol/l) for one to two days at RT together with triethylamine (8.0 eq.). The reaction mixture is diluted with ethyl acetate (EtOAc) and washed with 2 N hydrochloric acid. The organic phase is dried using MgSO₄, filtered and evaporated in vacuo.

The crude product is dissolved in acetone (0.01-0.1 mol/l) and treated with methyl iodide (40 eq.). The reaction mixture is stirred for 2 to 5 h at room temperature (RT) and then concentrated in vacuo.

The residue is dissolved in methanol (0.01-0.1 mol/l) and, for the preparation of the unsubstituted amidines, treated with ammonium acetate (3 eq.) and ammonium chloride (2 eq.). For the preparation of the substituted amidine derivatives, primary or secondary amines (1.5 eq.) and acetic acid (2 eq.) are added to the methanolic solution. After 5-30 h, the solvent is removed in vacuo and the residue is purified by chromatography on an RP8 silica gel column (water/acetonitrile 9/1-1/1+0.1% trifluoroacetic acid).

The following were prepared in an analogous manner:

### Example 31

### N-({3-[4-(2-Amino-2-iminoethyl)phenyl]-2-oxo-1,3-oxazolidin-5-yl}methyl)-5-chloro-2-thiophenecarboxamide

MS (ESI): m/z (%)=393 (M+H, 100);

HPLC (method 4): rt=2.63 min

### Example 32

### 5-Chloro-N-({3-[3-(4,5-dihydro-1H-imidazol-2-ylmethyl)phenyl]-2-oxo-1,3-oxazolidin-5-yl}methyl)-2-thiophenecarboxamide

MS (ESI): m/z (%)=419 (M+H, 100);

HPLC (method 4): rt=2.61 min

### Example 33

### 5-Chloro-N-[(3-{3-[2-imino-2-(4-morpholinyl)ethyl]phenyl}-2-oxo-1,3-oxazolidin-5-yl)methyl]-2-thiophenecarboxamide

MS (ESI): m/z (%)=463 (M+H, 100);

HPLC (method 4): rt=2.70 min

### Example 34

### 5-Chloro-N-[(3-{3-[2-imino-2-(1-pyrrolidinyl)ethyl]phenyl}-2-oxo-1,3-oxazolidin-5-yl)methyl]-2-thiophenecarboxamide

MS (ESI): m/z (%)=447 (M+H, 100);

HPLC (method 4): rt=2.82 min

### Example 35

### N-({3-[3-(2-Amino-2-iminoethyl)phenyl]-2-oxo-1,3-oxazolidin-5-yl}methyl)-5-chloro-2-thiophenecarboxamide

MS (ESI): m/z (%)=393 (M+H, 100);

HPLC (method 4): rt 2.60 min

### Example 140

### 5-Chloro-N-({3-[4-(4,5-dihydro-1H-imidazol-2-ylmethyl)phenyl]-2-oxo-1,3-oxazolidin-5-yl}methyl)-2-thiophenecarboxamide

MS (ESI): m/z (%)=419 (M+H, 100);

HPLC (method 4): rt=2.65 min

### Example 141

### 5-Chloro-N-[(3-{4-[2-imino-2-(4-morpholinyl)ethyl]phenyl}-2-oxo-1,3-oxazolidin-5-yl)methyl]-2-thiophenecarboxamide

MS (ESI): m/z (%)=463 (M+H, 100);

HPLC (method 4): rt=2.65 min

### Example 142

### 5-Chloro-N-[(3-{4-[2-imino-2-(1-piperidinyl)ethyl]phenyl}-2-oxo-1,3-oxazolidin-5-yl)methyl]-2-thiophenecarboxamide

MS (ESI): m/z (%)=461 (M+H, 100);

HPLC (method 4): rt=2.83 min

### Example 143

### 5-Chloro-N-[(3-{4-[2-imino-2-(1-pyrrolidinyl)ethyl]phenyl}-2-oxo-1,3-oxazolidin-5-yl)methyl]-2-thiophenecarboxamide

MS (ESI): m/z (%)=447 (M+H, 100);

HPLC (method 4): rt=2.76 min

### Example 144

### 5-Chloro-N-[(3-{4-[2-(cyclopentylamino)-2-iminoethyl]phenyl}-2-oxo-1,3-oxazolidin-5-yl)methyl]-2-thiophenecarboxamide

MS (ESI): m/z (%)=461 (M+H, 100);

HPLC (method 4): rt=2.89 min

### Example 145

### 5-Chloro-N-{[3-(4-{2-imino-2-[(2,2,2-trifluoroethyl)amino]ethyl}phenyl)-2-oxo-1,3-oxazolidin-5-yl]methyl}-2-thiophenecarboxamide

MS (ESI): m/z (%)=475 (M+H, 100);

HPLC (method 4): rt=2.79 min

### Example 146

### N-({3-[4-(2-Anilino-2-iminoethyl)phenyl]-2-oxo-1,3-oxazolidin-5-yl}methyl)-5-chloro-2-thiophenecarboxamide

MS (ESI): m/z (%)=469 (M+H, 100);

HPLC (method 4): rt=2.83 min

### Example 147

### 5-Chloro-N-[(3-{4-[2-imino-2-(2-pyridinylamino)ethyl]phenyl}-2-oxo-1,3-oxazolidin-5-yl)methyl]-2-thiophenecarboxamide

MS (ESI): m/z (%)=470 (M+H, 100);

HPLC (method 4): rt=2.84 min

The following Examples 148 to 151 relate to the cleavage of BOC amino-protective groups:

### General Method for the Cleavage of Boc Protective Groups (Tert-Butyloxycarbonyl):

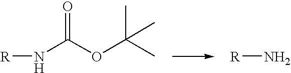

Aqueous trifluoroacetic acid (TFA, about 90%) is added dropwise to an ice-cooled solution of a tert-butyloxycarbonyl-(Boc)-protected compound in chloroform or dichloromethane (about 0.1 to 0.3 mol/l). After about 15 min, the ice cooling is removed and the mixture is stirred for about 2-3 h at room temperature, before concentrating the solution and drying it in a high vacuum. The residue is taken up in dichloromethane or dichloromethane/methanol and washed with saturated sodium hydrogencarbonate or 1N sodium hydroxide solution. The organic phase is washed with saturated sodium chloride solution, dried over a little magnesium sulfate and concentrated. Purification by crystallization from ether or ether/dichloromethane mixtures optionally takes place.

In an analogous manner, the following were prepared from the corresponding Boc-protected precursors:

### Example 148

### N-({3-[4-(Aminomethyl)phenyl]-2-oxo-1,3-oxazolidin-5-yl}methyl)-5-chloro-2-thiophenecarboxamide

starting from Example 92:

MS (ESI): m/z (%)=349 (M−NH₂, 25), 305 (100);

HPLC (method 1): rt (%)=3.68 (98).

IC₅₀: 2.2 μM

### Example 149

### N-{[3-(4-Aminophenyl)-2-oxo-1,3-oxazolidin-5-yl]methyl}-5-chloro-2-thiophenecarboxamide

starting from Example 93:

MS (ESI): m/z (%)=352 (M+H, 25);

HPLC (method 1): rt (%)=3.50 (100).

IC₅₀: 2 μM

An enantiomerically pure alternative synthesis of this compound is shown in the following scheme (cf also Delalande S. A., DE 2836305, 1979; Chem. Abstr. 90, 186926):

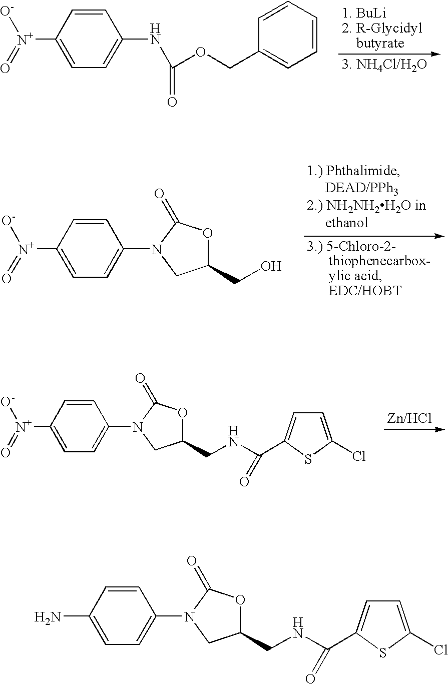

### Example 150

### 5-Chloro-N-({3-[4-(glycylamino)phenyl]-2-oxo-1,3-oxazolidin-5-yl}methyl)-2-thiophenecarboxamide

starting from Example 152:

MS (ES-pos): m/z (%)=408 (100);

HPLC (method 3): rt (%)=3.56 (97).

IC₅₀: 2 μM

### Example 151

### 5-(Aminomethyl)-3-[4-(2-oxo-1-pyrrolidinyl)phenyl]-1,3-oxazolidin-2-one

starting from Example 60:

MS (ESI): m/z (%)=276 (M+H, 100);

HPLC (method 3): rt (%)=2.99 (100).

IC₅₀: 2 μM

The following Examples 152 to 166 relate to the amino group derivatization of aniline- or benzylamine-substituted oxazolidinones with different reagents:

### Example 152

### 5-Chloro-N-({3-[4-(N-tert-butyloxycarbonylglycylamino)phenyl]-2-oxo-1,3-oxazolidin-5-yl}methyl)-2-thiophenecarboxamide

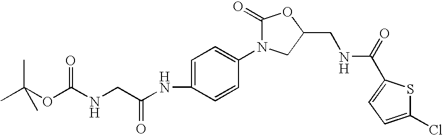

754 mg (2.1 mmol) of N-{[3-(4-aminophenyl)-2-oxo-1,3-oxazolidin-5-yl]methyl}-5-chloro-2-thiophenecarboxamide (from Example 149) are added at 0° C. to a solution of 751 mg (4.3 mmol) of Boc-glycine, 870 mg (6.4 mmol) of HOBT (1-hydroxy-1H-benzotriazole×H₂O), 1790 mg (4.7 mmol) of HBTU [o-(benzotriazol-1-yl)-N,N,N′,N′-tetramethyluronium hexafluorophosphate] and 1.41 ml (12.9 mmol) of N-methylmorpholine in 15 ml of DMF/CH₂Cl₂ (1:1). The mixture is stirred overnight at room temperature, before diluting with water. The precipitated solid is filtered off and dried. Yield: 894 mg (79.7% of theory);

MS (DCI, NH₃): m/z (%) 526 (M+NH₄, 100);

HPLC (method 3): rt (%)—-4.17 (97).

### Example 153

### N-[(3-{4-[(Acetylamino)methyl]phenyl}-2-oxo-1,3-oxazolidin-5-yl)methyl]-5-chloro-2-thiophenecarboxamide

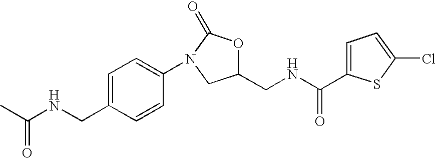

A mixture of 30 mg (0.082 mmol) of N-({3-[4-(aminomethyl)phenyl]-2-oxo-1,3-oxazolidin-5-yl}methyl)-5-chloro-2-thiophenecarboxamide (from Example 148) in 1.5 ml of absolute THF and 1.0 ml of absolute dichloromethane, 0.02 ml of absolute pyridine is treated at 0° C. with acetic anhydride (0.015 ml, 0.164 mmol). The mixture is stirred overnight at room temperature. The product is obtained by addition of ether and crystallization. Yield: 30 mg (87% of theory),

MS (ESI): m/z (%)-408 (M+H, 18), 305 (85);

HPLC (method 1): rt (%)=3.78 (97).

IC₅₀; 0.6 μM

### Example 154

### N-{[3-(4-{[(Aminocarbonyl)amino]methyl}phenyl)-2-oxo-1,3-oxazolidin-5-yl]methyl}-5-chloro-2-thiophenecarboxamide

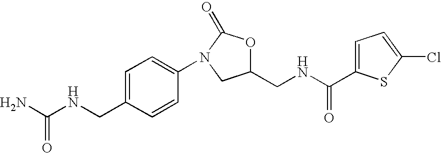

0.19 ml (0.82 mmol) of trimethylsilyl isocyanate is added dropwise at room temperature to a mixture of 30 mg (0.082 mmol) of N-({3-[4-(aminomethyl)phenyl]-2-oxo-1,3-oxazolidin-5-yl}methyl)-5-chloro-2-thiophenecarboxamide (from Example 148) in 1.0 ml dichloromethane. The mixture is stirred overnight, before obtaining the product by filtration after addition of ether. Yield: 21.1 mg (52% of theory),

MS (ESI): m/z (%)=409 (M+H, 5), 305 (72);

HPLC (method 1): rt (%)=3.67 (83).

IC₅₀: 1.3 μM

General Method for the Acylation of N-{[3-(4-aminophenyl)-2-oxo-1,3-oxazolidin-5-yl]methyl}-5-chloro-2-thiophenecarboxamide with Carbonyl Chlorides:

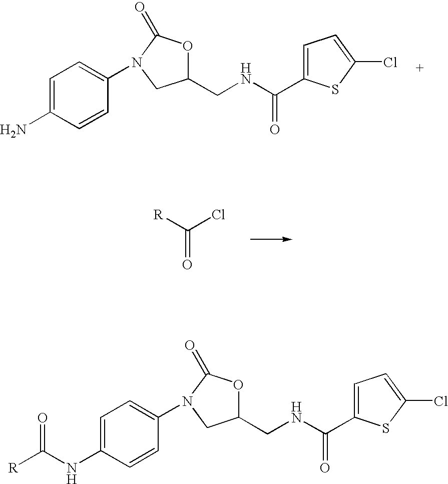

Under argon, an about 0.1 molar solution of N-{[3-(4-aminophenyl)-2-oxo-1,3-oxazolidin-5-yl]methyl}-5-chloro-2-thiophenecarboxamide (from Example 149) (1.0 eq.) in absolute dichloromethane/pyridine (19:1) is added dropwise to the appropriate acid chloride (2.5 eq.). The mixture is stirred overnight, before being treated with about 5 eq of PS-trisamine (Argonaut Technologies) and 2 ml of absolute dichloromethane. After gentle stirring for 1 h, it is filtered off and the filtrate is concentrated. A purification of the products by preparative RP-HPLC optionally takes place.

The following were prepared in an analogous manner:

### Example 155

### N-({3-[4-(Acetylamino)phenyl]-2-oxo-1,3-oxazolidin-5-yl}methyl)-5-chloro-2-thiophenecarboxamide

LC-MS: m/z (%)=394 (M+H, 100);

LC-MS (method 6): rt (%)=3.25 (100).

IC₅₀: 1.2 μM

### Example 156

### 5-Chloro-N-[(2-oxo-3-{4-[(2-thienylcarbonyl)amino]phenyl}-1,3-oxazolidin-5-yl)methyl]-2-thiophenecarboxamide

LC-MS: m/z (%) 462 (M+H, 100);

LC-MS (method 6): rt (%)=3.87 (100).

IC₅₀: 1.3 μM

### Example 157

### 5-Chloro-N-[(3-{4-[(methoxyacetyl)amino]phenyl}-2-oxo-1,3-oxazolidin-5-yl)methyl]-2-thiophenecarboxamide

LC-MS: m/z (%)=424 (MA-H, 100);

LC-MS (method 6): rt (%)=3.39 (100).

IC₅₀: 0.73 μM

### Example 158

### N-{4-[5-({[(5-Chloro-2-thienyl)carbonyl]-amino}methyl)-2-oxo-1,3-oxazolidin-3-yl]phenyl}-3,5-dimethyl-4-isoxazolecarboxamide

LC-MS: m/z (%)=475 (M+H, 100).

IC₅₀: 0.46 μM

### Example 159

### 5-Chloro-N-{[3-(4-{[(3-chloropropyl)sulfonyl]amino}phenyl)-2-oxo-1,3-oxazolidin-5-yl]methyl}-2-thiophenecarboxamide

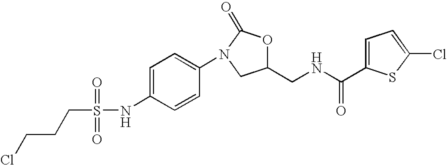

35 mg (0.1 mmol) of N-{[3-(4-aminophenyl)-2-oxo-1,3-oxazolidin-5-yl]methyl}-5-chloro-2-thiophenecarboxamide (from Example 149) are added to an ice-cooled solution of 26.4 mg (0.15 mmol) of 3-chloro-1-propanesulfonyl chloride and 0.03 ml (0.2 mmol) of triethylamine in 3.5 ml of absolute dichloromethane. After 30 min, the ice cooling is removed and the mixture is stirred overnight at room temperature, before adding 150 mg (about 5.5 eq) of PS-trisamine (Argonaut Technologies) and 0.5 ml of dichloromethane. The suspension is gently stirred for 2 h, filtered (the resin is washed with dichloromethane/methanol) and the filtrate is concentrated. The product is purified by preparative RP-HPLC. Yield: 19.6 mg (40% of theory),

LC-MS: m/z (%)=492 (M+H, 100);

LC-MS (method 5): rt (%)=3.82 (91).

IC₅₀: 1.7 μM

### Example 160

### 5-Chloro-N-({3-[4-(1,1-dioxido-2-isothiazolidinyl)phenyl]-2-oxo-1,3-oxazolidin-5-yl}methyl)-2-thiophenecarboxamide

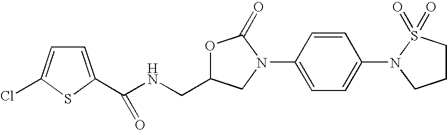

A mixture of 13.5 mg (0.027 mmol) of 5-chloro-N-{[3-(4-{[(3-chloro-propyl)sulfonyl]amino}phenyl)-2-oxo-1,3-oxazolidin-5-yl]methyl}-2-thiophenecarboxamide (from Example 159) and 7.6 mg (0.055 mmol) of potassium carbonate in 0.2 ml of DMF is heated for 2 h at 100° C. After cooling, it is diluted with dichloromethane and washed with water. The organic phase is dried and concentrated. The residue is purified by preparative thin layer chromatography (silica gel, dichloromethane/methanol, 95:5). Yield: 1.8 mg (14.4% of theory),

MS (ESI): m/z (%)=456 (M+H, 15), 412 (100);

LC-MS (method 4): rt (%)=3.81 (90).

IC₅₀: 0.14 μA

### Example 161

### 5-Chloro-N-[((5S)-3-{4-[(5-chloropentanoyl)amino]phenyl}-2-oxo-1,3-oxazolidin-5-yl)methyl]-2-thiophenecarboxamide

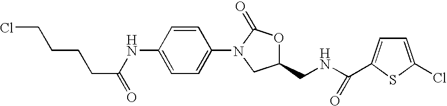

0.5 g (1.29 mmol) of N-{[(5S)-3-(4-aminophenyl)-2-oxo-1,3-oxazolidin-5-yl]methyl}-5-chloro-2-thiophenecarboxamide (from Example 149) are dissolved in 27 ml of tetrahydrofuran and treated with 0.2 g (1.29 mmol) of 5-chlorovaleryl chloride and also 0.395 ml (2.83 mmol) of triethylamine. The batch is evaporated in vacuo and chromatographed on silica gel using a toluene/ethyl acetate=1:1->ethyl acetate gradient. 315 mg (52% of theory) of a solid are obtained.

M.p.: 211° C.

### Example 162

### 5-Chloro-N-({(5S)-2-oxo-3-[4-(2-oxo-1-piperidinyl)phenyl]-1,3-oxazolidin-5-yl}methyl)-2-thiophenecarboxamide

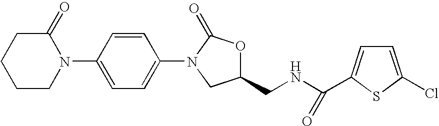

30 mg of 60 percent NaH in liquid paraffin are added under inert conditions to 5 ml of DMSO and the mixture is heated for 30 min to 75° C. until completion of the evolution of gas. Subsequently, a solution of 290 mg (0.617 mmol) of 5-chloro-N-[((5S)-3-{4-[(5-chloropentanoyl)amino]phenyl}-2-oxo-1,3-oxazolidin-5-yl)methyl]-2-thiophenecarboxamide (from Example 161) in 5 ml of methylene chloride is added and the mixture is stirred overnight at room temperature. The reaction is terminated and the mixture is added to 100 ml of water and extracted with ethyl acetate. The evaporated organic phase is chromatographed on an RP-8 column and eluted with acetonitrile/water. 20 mg (7.5% of theory) of the target compound are obtained.

M.p.: 205° C.;

NMR (300 MHz, d₆-DMSO): δ 1.85 (m, 4H), 2.35 (m, 2H), 3.58 (m, 4H), 3.85 (m, 1H), 4.2 (t, 1H), 4.82 (m, 1H), 7.18 (d, 1H, thiophene), 7.26 (d, 2H), 7.5 (d, 2H), 2.68 (d, 1H, thiophene), 9.0 (t, 1H, CONH).

IC₅₀: 2.8 nM

### Example 163

### 5-Chloro-N-[((5S)-3-{4-[(3-bromopropionyl)amino]phenyl}-2-oxo-1,3-oxazolidin-5-yl)methyl]-2-thiophenecarboxamide

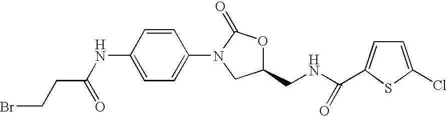

is obtained in an analogous manner from Example 149.

### Example 164

### 5-Chloro-N-({(5S)-2-oxo-3-[4-(2-oxo-1-azetidinyl)phenyl]-1,3-oxazolidin-5-yl}methyl)-2-thiophenecarboxamide

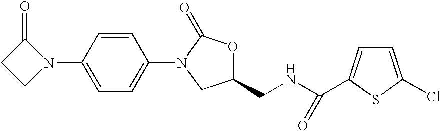

is obtained in an analogous manner by cyclization of the open-chain bromopropionyl compound from Example 163 by means of NaH/DMSO.

MS (ESI): m/z (%)=406 ([M+H]⁺, 100), Cl sample.

IC₅₀: 380 nM

### Example 165

### tert-Butyl 4-{-4-[5-({[(5-chloro-2-thienyl)carbonyl]amino}methyl)-2-oxo-1,3-oxazolidin-3-yl]phenyl}-3,5-dioxo-1-piperazinecarboxylate

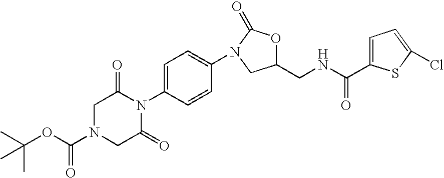

300 mg (0.85 mmol) of N-{[3-(4-aminophenyl)-2-oxo-1,3-oxazolidin-5-yl]methyl}-5-chloro-2-thiophenecarboxamide in 6 ml of a mixture of DMF and dichloromethane (1:1) are added to a solution of 199 mg (0.85 mmol) of Boc-iminodiacetic acid, 300 mg (2.2 mmol) of HOBT, 0.66 ml (6 mmol) of N-methylmorpholine and 647 mg (1.7 mmol) of HBTU. The mixture is stirred overnight, before, after diluting with dichloromethane, being washed with water, saturated ammonium chloride solution, saturated sodium hydrogencarbonate solution, water and saturated sodium chloride solution. The organic phase is dried over magnesium sulfate and concentrated. The crude product is purified by chromatography on silica gel (dichloromethane/methanol 98:2). Yield: 134 mg (29% of theory);

MS (ESI): m/z (%)=571 (M+Na, 82), 493 (100);

HPLC (method 3): rt (%)=4.39 (90).

IC₅₀: 2 μM

### Example 166

### N-[((5S)-3-{4-[(3R)-3-Amino-2-oxo-1-pyrrolidinyl]phenyl}-2-oxo-1,3-oxazolidin-5-yl)methyl]-5-chloro-2-thiophenecarboxamide trifluoroacetate

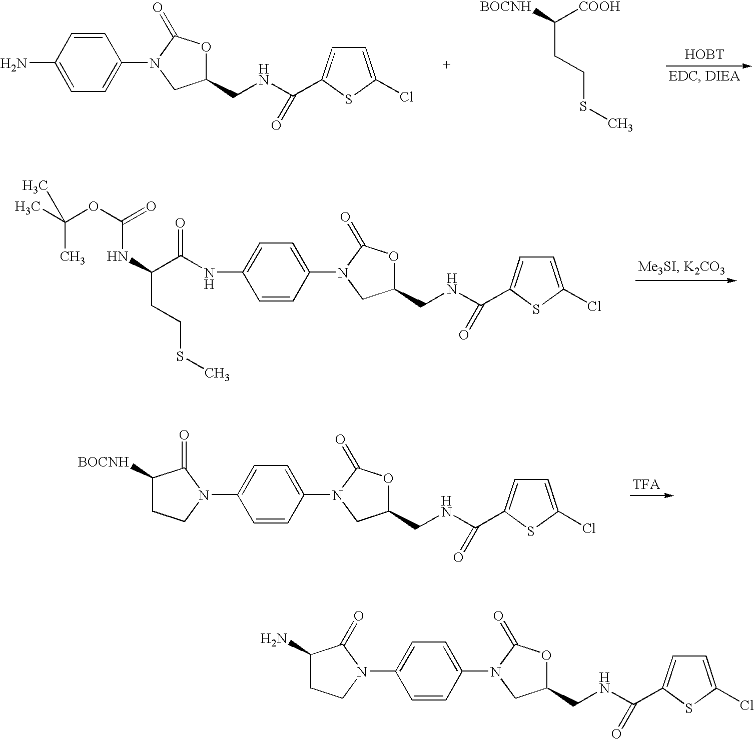

### N2-(tert-Butoxycarbonyl)-N1-{4-[(5S)-5-({[(5-chloro-2-thienyl)carbonyl]amino}methyl)-2-oxo-1,3-oxazolidin-3-yl]phenyl}-D-methioninamide

429 mg (1.72 mmol) of N-BOC-D-methionine, 605 mg (1.72 mmol) of N-{[(5S)-3-(4-aminophenyl)-2-oxo-1,3-oxazolidin-5-yl]methyl}-5-chloro-2-thiophenecarboxamide, and 527 mg (3.44 mmol) of HOBT hydrate are dissolved in 35 ml of DMF, and treated with 660 mg (3.441 mmol) of EDCI hydrochloride and subsequently dropwise with 689 mg (5.334 mmol) of N-ethyl-diisopropylamine. The mixture is stirred at room temperature for two days. The suspension obtained is filtered off with suction and the residue is washed with DMF. The combined filtrates are treated with some silica gel, evaporated in vacuo and chromatographed on silica gel using a toluene−>T10EE7 gradient. 170 mg (17% of theory) of the target compound having a melting point of 183° C. are obtained.

R_{f} (SiO₂, toluene/ethyl acetate=1:1):0.2.

¹H-NMR (300 MHz, d₆-DMSO): δ=1.4 (s, 1H, BOC), 1.88-1.95 (m, 2H), 2.08 (s, 3H, SMe), 2.4-2.5 (m, 2H, partially masked by DMSO), 3.6 (m, 2H), 3.8 (m, 1H), 4.15 (m, 2H), 4.8 (m, 1H), 7.2 (1H, thiophene), 7.42 (d, part of an AB system, 2H), 7.6 (d, part of an AB system, 2H), 7.7 (d, 1H, thiophene), 8.95 (t, 1H, CH₂NHCO), 9.93 (bs, 1H, NH).

### tert-Butyl (3R)-1-{4-[(5S)-5-({[(5-chloro-2-thienyl)carbonyl]amino}methyl)-2-oxo-1,3-oxazolidin-3-yl]phenyl}-2-oxo-3-pyrrolidinylcarbamate

170 mg (0.292 mmol) of N2-(tert-butoxycarbonyl)-N1-{4-[(5S)-5-({[(5-chloro-2-thienyl)carbonyl]amino}methyl)-2-oxo-1,3-oxazolidin-3-yl]phenyl}-D-methioninamide are dissolved in 2 ml of DMSO and treated with 178.5 mg (0.875 mmol) of trimethylsulfonium iodide and also 60.4 mg (0.437 mmol) of potassium carbonate and stirred for 3.5 hours at 80° C. Subsequently, the mixture is evaporated in a high vacuum and the residue is washed with ethanol. 99 mg of the target compound remain.

¹H-NMR (300 MHz, d₆-DMSO): δ=1.4 (s, 1H, BOC), 1.88-2.05 (m, 1H), 2.3-2.4 (m, 1H), 3.7-3.8 (m, 3H), 3.8-3.9 (m, 1H), 4.1-4.25 (m, 1H), 4.25-4.45 (m, 1H), 4.75-4.95 (m, 1H), 7.15 (1H, thiophene), 7.25 (d, 1H), 7.52 (d, part of an AB system, 2H), 7.65 (d, part of an AB system, 2H), 7.65 (d, 1H, thiophene), 9.0 (broad s, 1H).

### N-[((5S)-3-{4-[(3R)-3-Amino-2-oxo-1-pyrrolidinyl]phenyl}-2-oxo-1,3-oxazolidin-5-yl)methyl]-5-chloro-2-thiophenecarboxamide trifluoroacetate

97 mg (0.181 mmol) of tert-butyl (3R)-1-{4-[(5S)-5-({[(5-chloro-2-thienyl)carbonyl]amino}methyl)-2-oxo-1,3-oxazolidin-3-yl]phenyl}-2-oxo-3-pyrrolidinyl-carbamate are suspended in 4 ml of methylene chloride, 1.5 ml of trifluoroacetic acid are added and the mixture is stirred for 1 hour at room temperature. Subsequently, it is evaporated in vacuo and purified on an RP-HPLC (acetonitrile/water/0.1% TFA gradient). After evaporating the fraction concerned, 29 mg (37% of theory) of the target compound having a melting point of 241° C. (des.) are obtained.

R_{f} (SiO₂, EtOH/TEA-17:1) 0.19.

¹H-NMR (300 MHz, d₆-DMSO): δ=1.92-2.2 (m, 1H), 2.4-2.55 (m, 1H, partially masked by DMSO peak), 3.55-3.65 (m, 2H), 3.75-3.95 (m, 3H), 4.1-4.3 (m, 2H), 4.75-4.9 (m, 1H), 7.2 (1H, thiophene), 7.58 (d, part of an AB system, 2H), 7.7 (d, part of an AB system, 2H), 7.68 (d, 1H, thiophene), 8.4 (broad s, 3H, NH3), 8.9 (t, 1H, NHCO).

The following Examples 167 to 170 relate to the introduction of sulfonamide groups into phenyl-substituted oxazolidinones:

General Method for the Preparation of Substituted Sulfonamides Starting from 5-chloro-N-[(2-oxo-3-phenyl-1,3-oxazolidin-5-yl)methyl]-2-thiophenecarboxamide

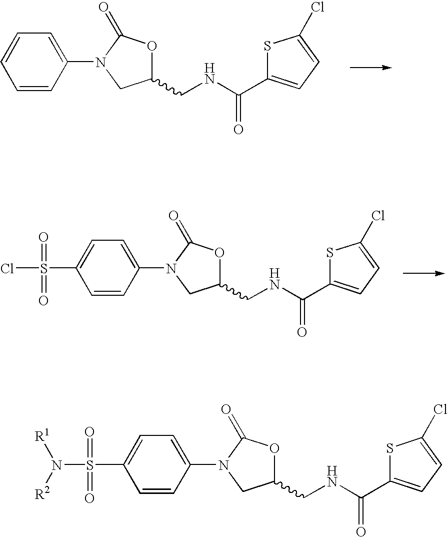

5-Chloro-N-[(2-oxo-3-phenyl-1,3-oxazolidin-5-yl)methyl]-2-thiophenecarboxamide (from Example 96) is added under argon at 5° C. to chlorosulfonic acid (12 eq.). The reaction mixture is stirred at room temperature for 2 h and subsequently added to ice water. The precipitate deposited is filtered, washed with water and dried.

Subsequently, it is dissolved in tetrahydrofuran (0.1 mol/l) under argon at room temperature and treated with the corresponding amine (3 eq.), triethylamine (1.1 eq.) and dimethylaminopyridine (0.1 eq.). The reaction mixture is stirred for 1-2 h and subsequently concentrated in vacuo. The desired product is purified by means of flash chromatography (dichloromethane-methanol mixtures).

The following were prepared in an analogous manner:

### Example 167

### 5-Chloro-N-({2-oxo-3-[4-(1-pyrrolidinylsulfonyl)phenyl]-1,3-oxazolidin-5-yl}methyl)-2-thiophenecarboxamide

MS (ESI): m/z (%)=492 ([M+Na]⁺, 100), 470 ([M+H]⁺, 68), Cl sample;

HPLC (method 3): rt (%)=4.34 (100).

IC₅₀: 0.5

### Example 168

### 5-Chloro-N-[(3-{4-[(4-methyl-1-piperazinyl)sulfonyl]phenyl}-2-oxo-1,3-oxazolidin-5-yl)methyl]-2-thiophenecarboxamide

MS (ESI): m/z (%)=499 ([M+H]⁺, 100), Cl sample;

HPLC (method 2): rt (%)=3.3 (100).

### Example 169

### 5-Chloro-N-({2-oxo-3-[4-(1-piperidinylsulfonyl)phenyl]-1,3-oxazolidin-5-yl}methyl)-2-thiophenecarboxamide

MS (ESI): m/z (%)=484 ([M+H]⁺, 100), Cl sample;

HPLC (method 2): rt (%)=4.4 (100).

### Example 170

### 5-Chloro-N-[(3-{4-[(4-hydroxy-1-piperidinyl)sulfonyl]phenyl}-2-oxo-1,3-oxazolidin-5-yl)methyl]-2-thiophenecarboxamide

MS (ESI): m/z (%)=500 ([M+H]⁺, 100), Cl sample;

HPLC (method 3): rt (%)=3.9 (100).

### Example 171

### 5-Chloro-N-({2-oxo-3-[4-(1-pyrrolidinyl)phenyl]-1,3-oxazolidin-5-yl}methyl)-2-thiophenecarboxamide

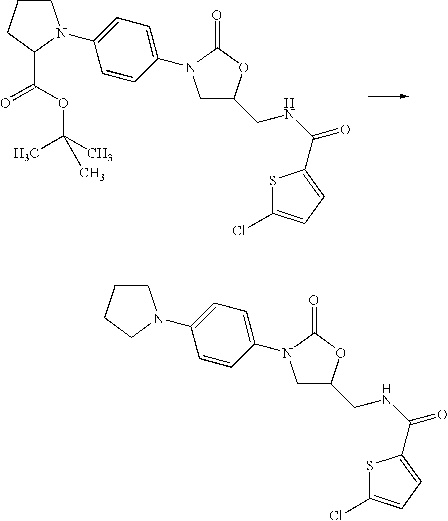

780 mg (1.54 mmol) of tert-butyl-1-{4-[5-({[(5-chloro-2-thienyl)carbonyl]amino}methyl)-2-oxo-1,3-oxazolidin-3-yl]phenyl}prolinate are dissolved in 6 ml of dichloromethane and 9 ml of trifluoroacetic acid and the mixture is stirred for two days at 40° C. The reaction mixture is then concentrated and stirred with ether and 2 N sodium hydroxide solution. The aqueous phase is concentrated and stirred with ether and 2 N hydrochloric acid. The organic phase from this extraction is dried over MgSO₄, filtered and concentrated. The crude product is chromatographed on silica gel (CH₂Cl₂/EtOH/conc. aq. NH₃ soln.=100/1/0.1 to 20/1/0.1).

280 mg (40% of theory) of the product are obtained.

MS (ESI): m/z (%)=406 (M+H, 100);

HPLC (method 4): rt=3.81 min.

### HPLC Parameters and LC-MS Parameters of the HPLC and LC-MS Data Indicated in the Preceding Examples (the Unit of the Retention Time (rt) is Minutes):

[1] Column: Kromasil C18, L-R temperature: 30° C., flow=0.75 mlmin⁻¹, eluent: A=0.01 M HClO₄, B=CH₃CN, gradient:−>0.5 min 98% A−>4.5 min 10% A−>6.5 min 10% A
[2] Column: Kromasil C18 60*2, L-R temperature: 30° C., flow=0.75 mlmin⁻¹, eluent: A=0.01 M H₃PO₄, B=CH₃CN, gradient:−>0.5 min 90% A−>4.5 min 10% A−>6.5 min 10% A
[3] Column: Kromasil C18 60*2, L-R temperature: 30° C., flow=0.75 mlmin⁻¹, eluent: A=0.005 M HClO₄, B=CH₃CN, gradient:−>0.5 min 98% A−>4.5 min 10% A−>6.5 min 10% A
[4] Column: Symmetry C18 2.1×150 mm, column oven: 50° C., flow=0.6 mlmin⁻¹, eluent: A=0.6 g of 30% strength HCl/1 water, B=CH₃CN, gradient: 0.0 min 90% A−>4.0 min 10% A−>9 min 10% A

### [5] MHZ-2Q, Instrument Micromass Quattro LCZ

Column Symmetry C18, 50 mm×2.1 mm, 3.5 μm, temperature: 40° C., flow=0.5 ml min⁻¹, eluent A=CH₃CN+0.1% formic acid, eluent B=water+0.1% formic acid, gradient: 0.0 min 10% A−>4 min 90% A−>6 min 90% A

### [6] MHZ-2P, Instrument Micromass Platform LCZ

Column Symmetry C18, 50 mm×2.1 mm, 3.5 μm, temperature: 40° C., flow=0.5 eluent A=CH₃CN+0.1% formic acid, eluent B=water+0.1% formic acid, gradient: 0.0 min 10% A−>4 min 90% A−>6 min 90% A

### [7] MHZ-7Q, Instrument Micromass Quattro LCZ

Column Symmetry C18, 50 mm×2.1 mm, 3.5 μm, temperature: 40° C., flow=0.5 mlmin⁻¹, eluent A=CH₃CN+0.1% formic acid, eluent B=water+0.1% formic acid, gradient: 0.0 min 5% A−>1 min 5% A−>5 min 90% A−>6 min 90% A

### General Method for Preparation of Oxazolidinones of the General Formula B by Solid Phase-Assisted Synthesis

Reactions with different resin-bound products took place in a set of separate reaction vessels.

5-(Bromomethyl)-3-(4-fluoro-3-nitrophenyl)-1,3-oxazolidin-2-one A (prepared from epibromohydrin and 4-fluorine-3-nitrophenyl isocyanate using LiBr/Bu₃PO in xylene analogously to U.S. Pat. No. 4,128,654, Ex. 2) (1.20 g, 3.75 mmol) and ethyldiisopropylamine (DIEA, 1.91 ml, 4.13 mmol) were dissolved in DMSO (70 ml), treated with a secondary amine (1.1 eq, amine component 1) and reacted for 5 h at 55° C. TentaGel SAM resin (5.00 g, 0.25 mmol/g) was added to this solution and reacted for 48 h at 75° C. The resin was filtered and repeatedly washed with methanol (MeOH), dimethylformamide (DMF), MeOH, dichloromethane (DCM) and diethyl ether and dried. The resin (5.00 g) was suspended in dichloromethane (80 ml), treated with DIEA eq) and 5-chlorothiophene-2-carbonyl chloride [prepared by reaction of 5-chlorothiophene-2-carboxylic acid (5 eq) and 1-chloro-1-dimethylamino-2-methylpropene (5 eq) in DCM (20 ml) at room temperature for 15 minutes] and reacted for 5 h at room temperature. The resin obtained was filtered and repeatedly washed with MeOH, DCM and diethyl ether and dried. Subsequently, the resin was suspended in DMF/water (v/v 9:2, 80 ml), treated with SnCl₂*2H₂O (5 eq) and reacted for 18 h at room temperature. The resin was in turn repeatedly washed with MeOH, DMF, water, MeOH, DCM and diethyl ether and dried. This resin was suspended in DCM, treated with DIEA (10 eq) and at 0° C. with an acid chloride (5 eq of acid derivative 1) and reacted at room temperature overnight. Carboxylic acids were converted into the corresponding acid chlorides before the reaction by reaction with 1-dimethylamino-1-chloro-2-methylpropene (1 eq, based on the carboxylic acid) in DCM at room temperature for 15 min. The resin was repeatedly washed with DMF, water, DMF, MeOH, DCM and diethyl ether and dried. In the case of the use of Fmoc-protected amino acids as acid derivative 1, the Fmoc protective group was cleaved at room temperature for 15 minutes in the last reaction step by reaction with piperidine/DMF (v/v, 1/4) and the resin was washed with DMF, MeOH, DCM and diethyl ether and dried. The products were subsequently cleaved from the solid phase using trifluoroacetic acid (TFA)/DCM (v/v, 1/1), the resin was filtered off and the reaction solutions were evaporated. The crude products were filtered through silica gel (DCM/MeOH, 9:1) and evaporated in order to obtain a set of products B.

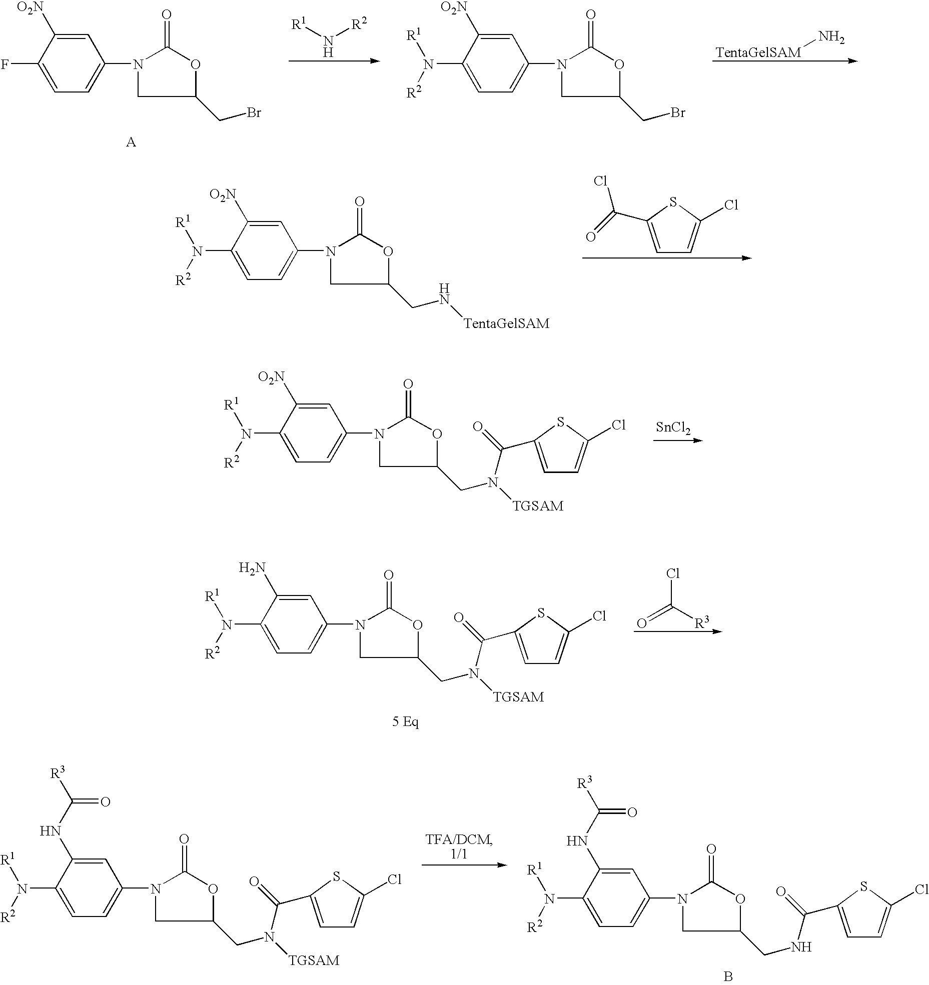

Compounds prepared by solid phase-assisted synthesis:

### Example 172

### N-({3-[3-Amino-4-(1-pyrrolidinyl)phenyl]-2-oxo-1,3-oxazolidin-5-yl}methyl)-5-chloro-2-thiophenecarboxamide

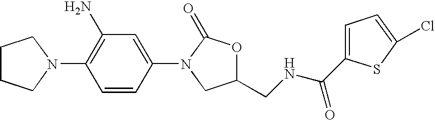

Analogously to the general working procedure for the preparation of the derivatives B, 5 g (1.25 mmol) of TentaGel SAM resin were reacted with pyrrolidine as amine derivative 1. The aniline obtained after the reaction with SnCl₂*2H₂O was cleaved from the solid phase without a further acylation step and evaporated. The crude product was partitioned between ethyl acetate and NaHCO₃ solution, the organic phase was salted out using NaCl, decanted and evaporated to dryness. This crude product was purified by vacuum flash chromatography on silica gel (dichloromethane/ethyl acetate, 3:1-1:2).

¹H-NMR (300 MHz, CDCl₃): 1.95-2.08, br, 4H, 3.15-3.30, br, 4H, 3.65-3.81, m, 2H, 3.89, ddd, 1H, 4.05, dd, 1H, 4.81, dddd, 1H, 6.46, dd, 1H, 6.72, dd, 1H, 6.90, dd, 1H, 6.99, dd, 1H, 7.03, dd, 1H, 7.29, d, 1H.

### Example 173

### N-[(3-{3-(β-Alanylamino)-4-[(3-hydroxypropyl)amino]phenyl}-2-oxo-1,3-oxazolidin-5-yl)methyl]-5-chloro-2-thiophenecarboxamide

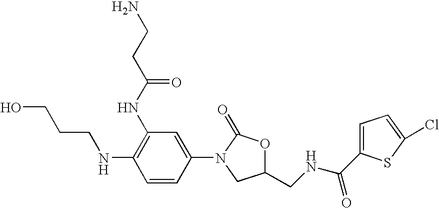

Analogously to the general working procedure for the preparation of the derivatives B, 5 g (1.25 mmol) of TentaGel SAM resin were reacted with azetidine as amine derivative 1 and Fmoc-β-alanine as acid derivative 1. The crude product obtained after the cleavage was stirred for 48 h in methanol at room temperature and evaporated to dryness. This crude product was purified by reversed phase HPLC using a water/TFA/acetonitrile gradient.

¹H-NMR (400 MHz, CD₃OD): 2.31, tt, 2H, 3.36, t, 2H, 3.54, t, 2H, 3.62, t, 2H, 3.72, dd, 1H, 3.79, dd, 1H, 4.01, dd, 1H, 4.29, dd, 2H, 4.43, t, 2H, 4.85-4.95, m, 1H, 7.01, d, 1H, 4.48-7.55, m, 2H, 7.61, d, 1H, 7.84, d, 1H.

### Example 174

### N-({3-[4-(3-Amino-1-pyrrolidinyl)-3-nitrophenyl]-2-oxo-1,3-oxazolidin-5-yl}methyl)-5-chloro-2-thiophenecarboxamide

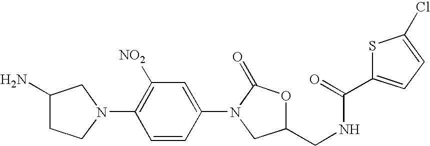

Analogously to the general general working procedure for the preparation of the derivatives B, 130 mg (32.5 μmol) of TentaGel SAM resin were reacted with tert-butyl 3-pyrrolidinylcarbamate as amine derivative 1. The nitrobenzene derivative obtained after the acylation with 5-chlorothiophenecarboxylic acid was cleaved from the solid phase and evaporated. This crude product was purified by reversed phase HPLC using a water/TFA/acetonitrile gradient.

¹H-NMR (400 MHz, CD₃OH): 2.07-2.17, m, 1H, 2.39-2.49, m, 1H, 3.21-3.40, m, 2H, 3.45, dd, 1H, 3.50-3.60, m, 1H, 3.67, dd, 1H, 3.76, dd, 1H, 3.88-4.00, m, 2H, 4.14-4.21, t, 1H, 4.85-4.95, m, 1H, 7.01, d, 1H, 7.11, d, 1H, 7.52, d, 1H, 7.66, dd, 1H, 7.93, d, 1H.

### Example 175

### N-({3-[3-Amino-4-(1-piperidinyl)phenyl]-2-oxo-1,3-oxazolidin-5-yl}methyl)-5-chloro-2-thiophenecarboxamide

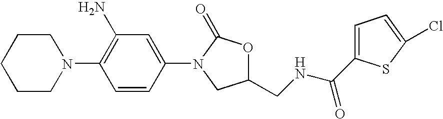

Analogously to the general working procedure for the preparation of the derivatives B, 130 mg (32.5 μmol) of TentaGel SAM resin were reacted with piperidine as amine derivative 1. The aniline obtained after the reduction was cleaved from the solid phase without a further acylation step and evaporated. This crude product was purified by reversed phase HPLC using a water/TFA/acetonitrile gradient.

¹H-NMR (400 MHz, CD₃OH): 1.65-1.75, m, 2H, 1.84-1.95, m, 4H, 3.20-3.28, m, 4H; 3.68, dd, 1H, 3.73, dd, 1H, 3.90, dd, 1H, 4.17, dd, 1H, 4.80-4.90, m, 1H, 7.00, d, 1H, 7.05, dd, 1H, 7.30-7.38, m, 2H, 7.50, d, 1H.

### Example 176

### N-({3-[3-(Acetylamino)-4-(1-pyrrolidinyl)phenyl]-2-oxo-1,3-oxazolidin-5-yl}methyl)-5-chloro-2-thiophenecarboxamide

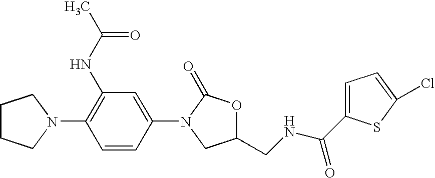

Analogously to the general working procedure for the preparation of the derivatives B, 130 mg (32.5 μmol) of TentaGel SAM resin were reacted with pyrrolidine as amine derivative 1 and acetyl chloride as acid derivative 1. The crude product was partitioned between ethyl acetate and NaHCO₃ solution, the organic phase was salted out using NaCl, decanted and evaporated to dryness. This crude product was purified by vacuum flash chromatography on silica gel (dichloromethane/ethyl acetate, 1:1-0:1).

¹H-NMR (400 MHz, CD₃OH): 1.93-2.03, br, 4H, 2.16, s, 3H; 3.20-3.30, br, 4H, 3.70, d, 2H, 3.86, dd, 1H, 4.10, dd, 1H, 4.14, dd, 1H, 4.80-4.90, m, 1H, 7.00, d, 1H, 7.07, d, 1H, 7.31, dd, 1H, 7.51, d, 1H, 7.60, d, 1H.

The following compounds were prepared analogously to the general working procedure.

| | | Ret. | HPLC |
|---|---|---|---|
| Example | Structure | time | [%] |
| 177 | 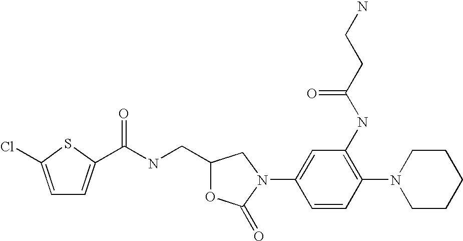 | 2.62 | 79.7 |
| 178 | 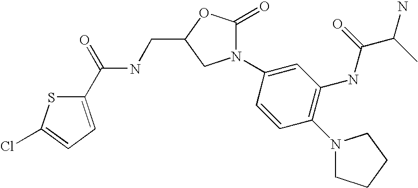 | 2.49 | 33.7 |
| 179 | 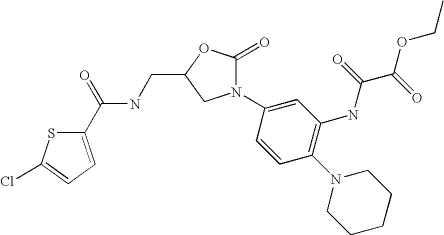 | 4.63 | 46.7 |
| 180 | 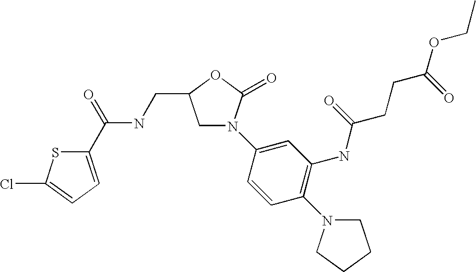 | 3.37 | 44.8 |
| 181 | 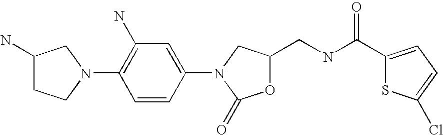 | 2.16 | 83 |
| 182 | 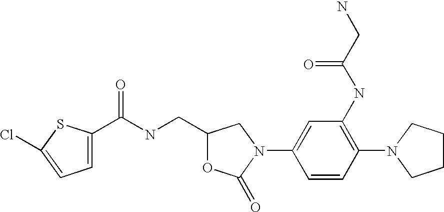 | 2.31 | 93.3 |
| 183 | 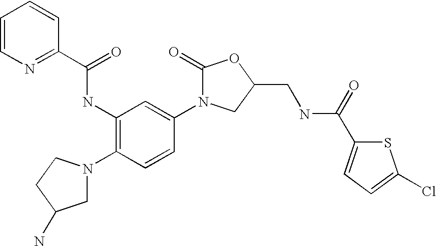 | 2.7 | 100 |
| 184 | 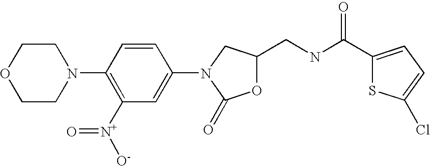 | 3.91 | 51 |
| 185 | 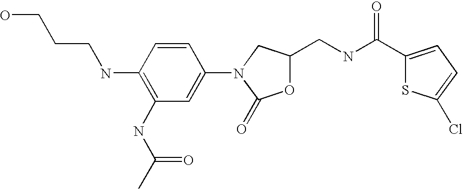 | 2.72 | 75.2 |
| 186 | 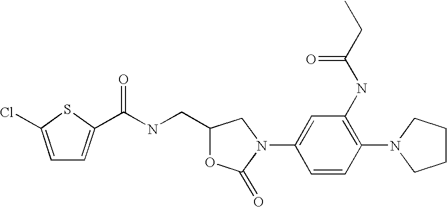 | 3.17 | 46 |
| 187 | 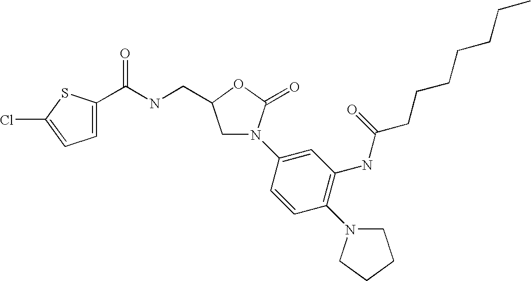 | 4.61 | 50.2 |
| 188 | 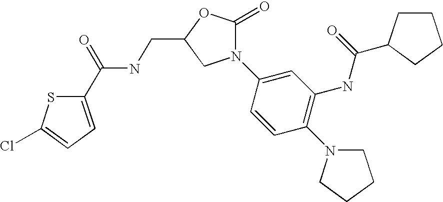 | 3.89 | 56.6 |
| 189 | 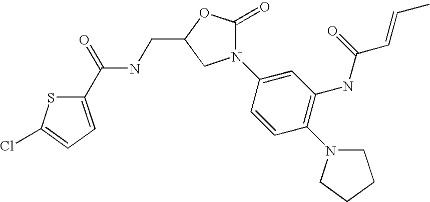 | 3.37 | 52.9 |
| 190 | 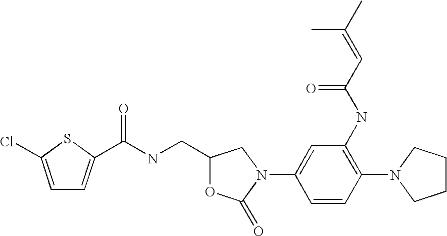 | 3.6 | 63.9 |
| 191 | 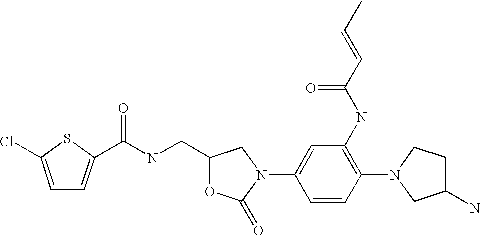 | 2.52 | 70.1 |
| 192 | 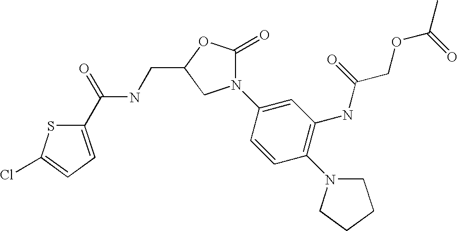 | 3.52 | 46.6 |
| 193 | 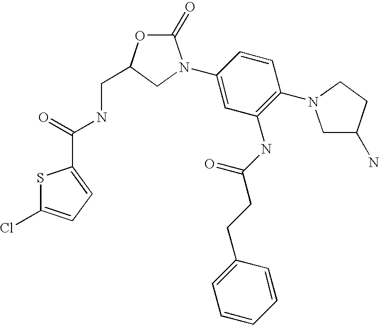 | 2.87 | 50.1 |
| 194 | 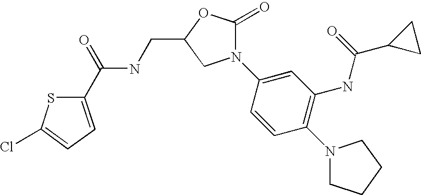 | 3.25 | 71.1 |
| 195 | 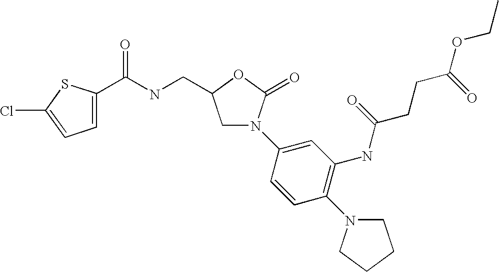 | 2.66 | 67 |
| 196 | 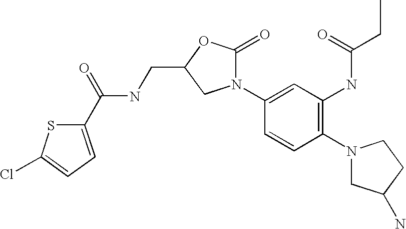 | 2.4 | 52.1 |
| 197 | 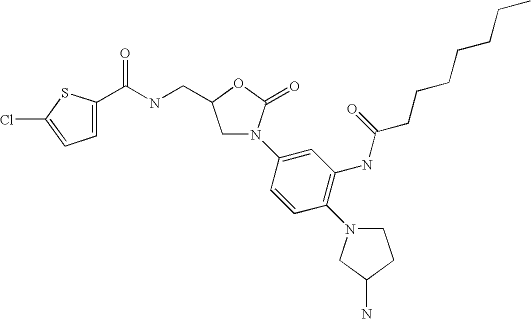 | 3.13 | 48.9 |
| 198 | 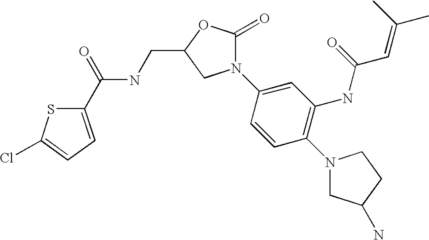 | 2.67 | 75.5 |
| 199 | 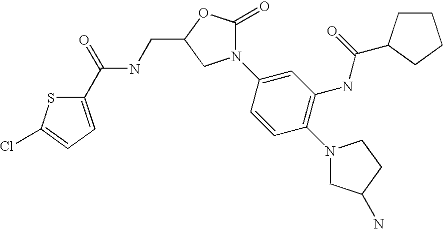 | 2.72 | 65.7 |
| 200 | 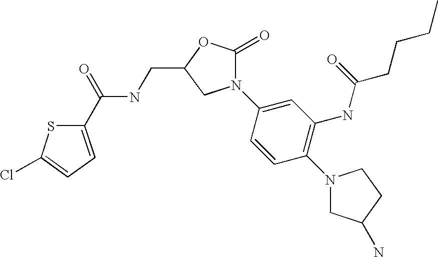 | 2.71 | 57.3 |
| 201 | 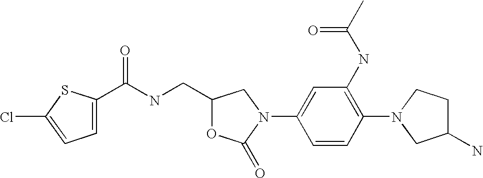 | 2.22 | 100 |
| 202 | 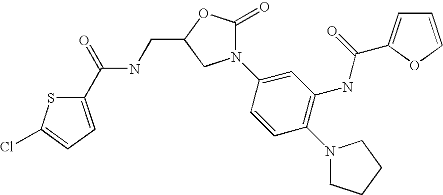 | 3.89 | 75.7 |
| 203 | 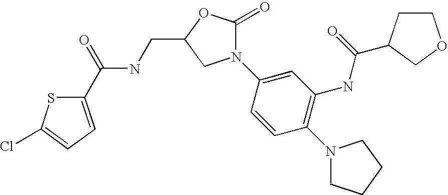 | 3.19 | 49.6 |
| 204 | 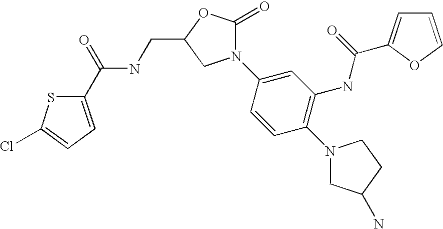 | 2.55 | 88.2 |
| 205 | 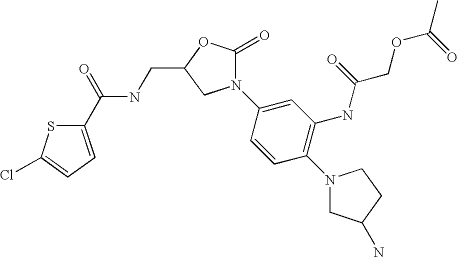 | 2.44 | 68.6 |
| 206 | 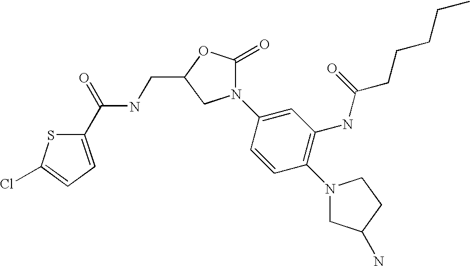 | 2.86 | 71.8 |
| 207 | 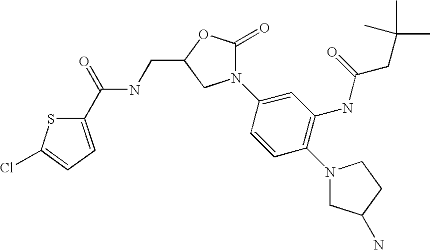 | 2.8 | 63.6 |
| 208 | 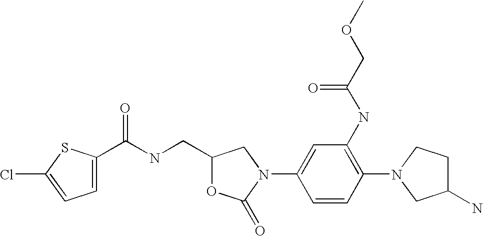 | 2.41 | 77 |
| 209 | 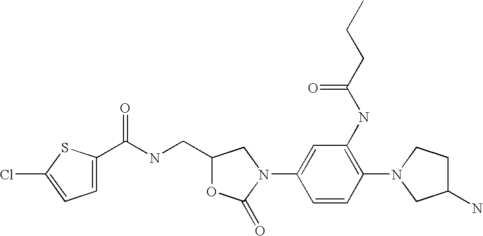 | 2.56 | 67.9 |
| 210 | 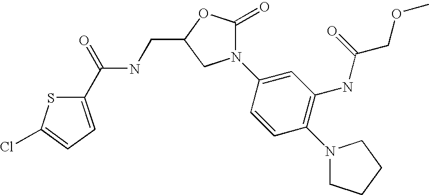 | 3.67 | 78.4 |
| 211 | 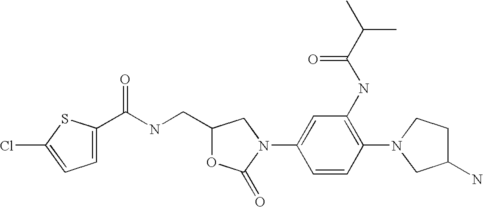 | 2.54 | 69.8 |
| 212 | 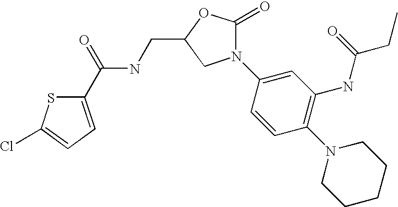 | 3.84 | 59.2 |
| 213 | 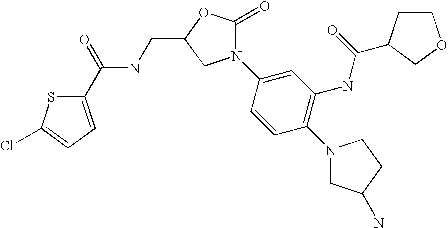 | 2.41 | 67.8 |
| 214 | 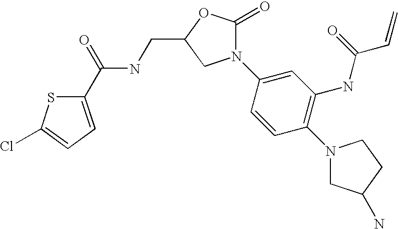 | 2.41 | 75.4 |
| 215 | 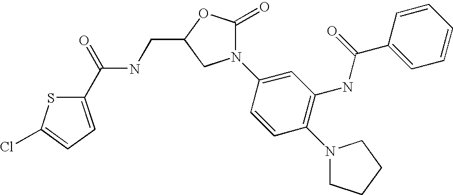 | 4.01 | 81.3 |
| 216 | 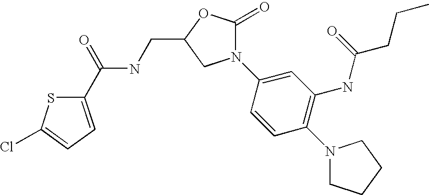 | 3.46 | 49.5 |
| 217 | 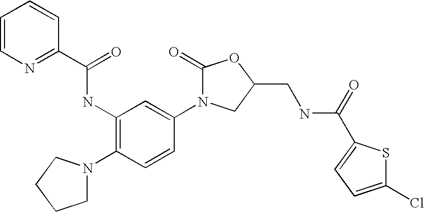 | 4.4 | 60.2 |
| 218 | 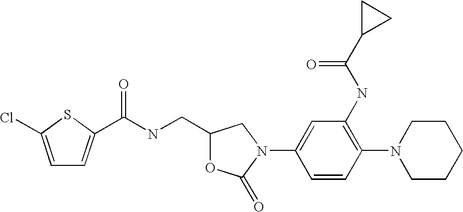 | 3.79 | 70.9 |
| 219 | 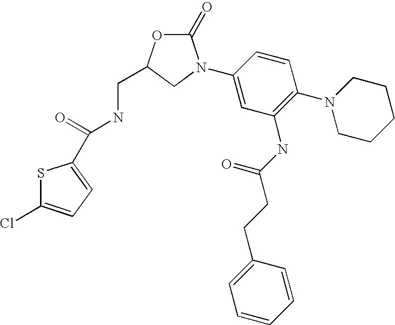 | 4.57 | 51.5 |
| 220 | 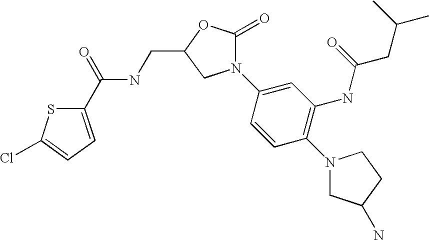 | 2.68 | 100 |
| 221 | 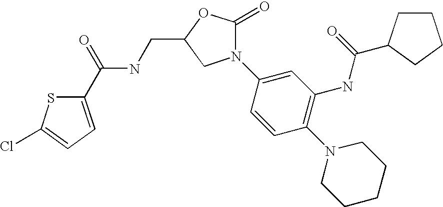 | 4.53 | 63.5 |
| 222 | 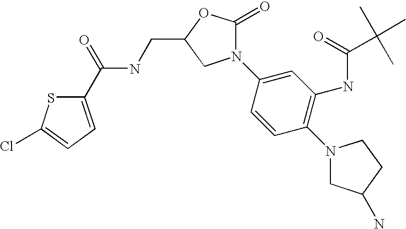 | 2.66 | 89.2 |
| 223 | 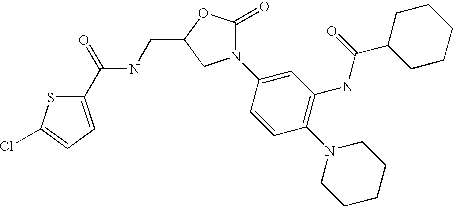 | 4.76 | 69.3 |
| 224 | 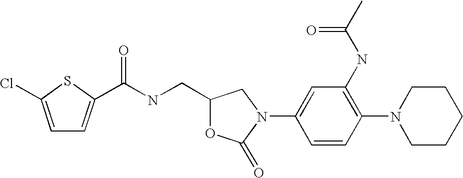 | 3.45 | 77.4 |
| 225 | 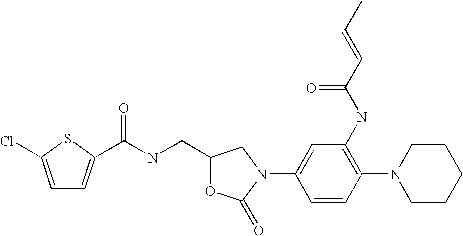 | 3.97 | 63.2 |
| 226 | 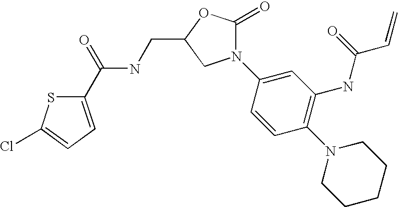 | 3.94 | 61.4 |
| 227 | 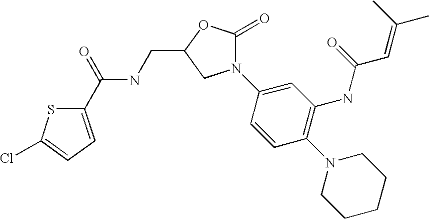 | 4.15 | 66.3 |
| 228 | 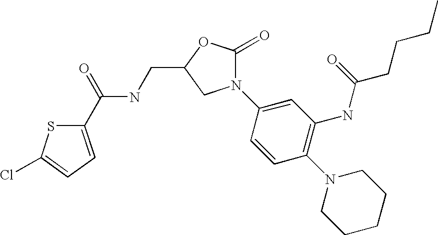 | 4.41 | 55.1 |
| 229 | 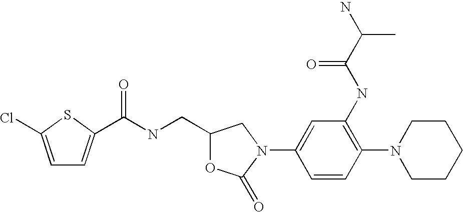 | 2.83 | 41.1 |
| 230 | 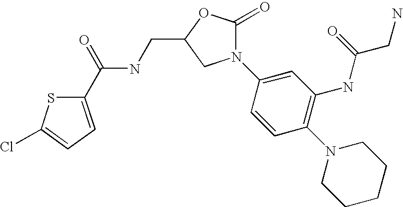 | 2.7 | 83 |
| 231 | 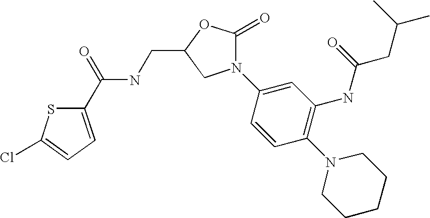 | 4.39 | 64.2 |
| 232 | 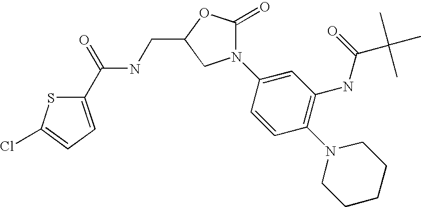 | 4.85 | 74.9 |
| 233 | 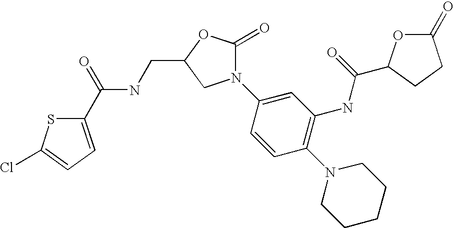 | 4.17 | 41 |
| 234 | 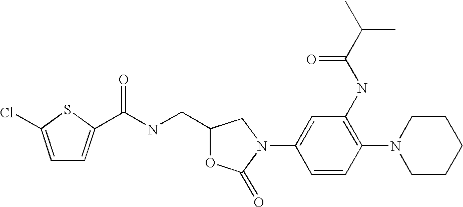 | 4.21 | 61.8 |
| 235 | 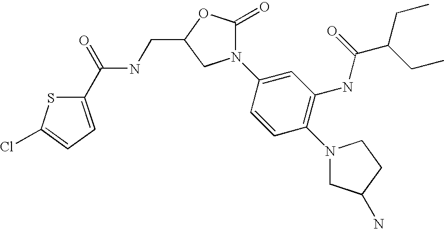 | 2.75 | 100 |
| 236 | 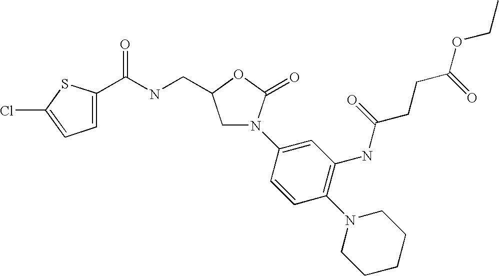 | 3.94 | 50 |
| 237 | 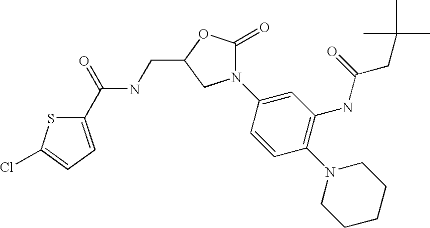 | 4.65 | 75.8 |
| 238 | 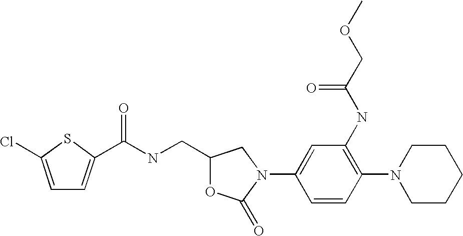 | 4.4 | 75.3 |
| 239 | 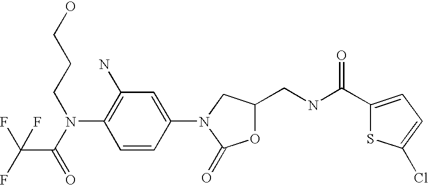 | 4.24 | 62.2 |
| 240 | 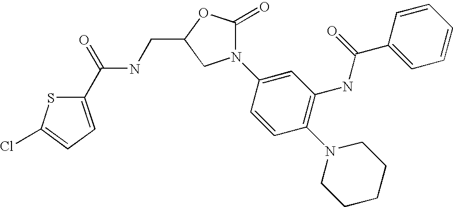 | 4.76 | 75.1 |
| 241 | 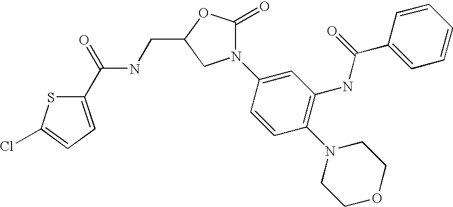 | 4.17 | 72.5 |
| 242 | 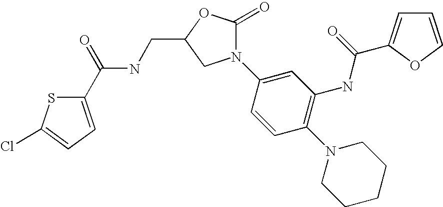 | 4.6 | 74.8 |
| 243 | 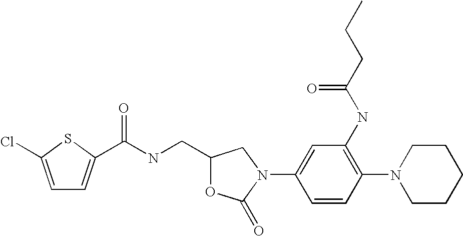 | 4.12 | 51.6 |
| 244 | 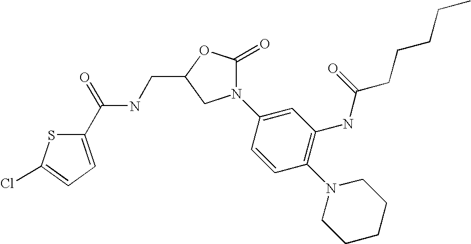 | 4.71 | 66.2 |
| 245 | 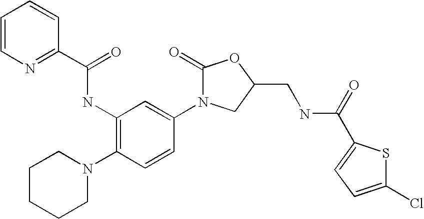 | 4.86 | 62 |
| 246 | 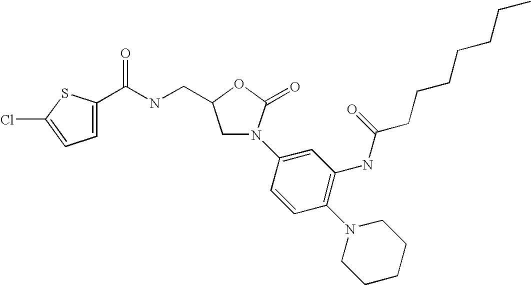 | 5.23 | 58.3 |
| 247 | 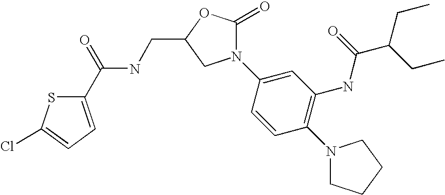 | 4.17 | 72.4 |
| 248 | 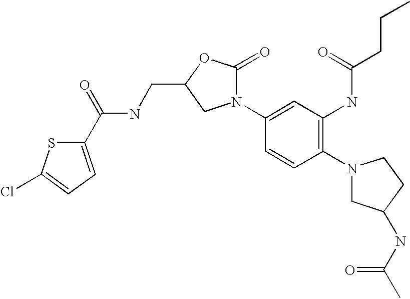 | 3.35 | 59.6 |
| 249 | 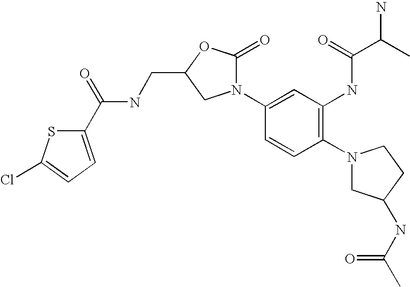 | 2.41 | 60.3 |
| 250 | 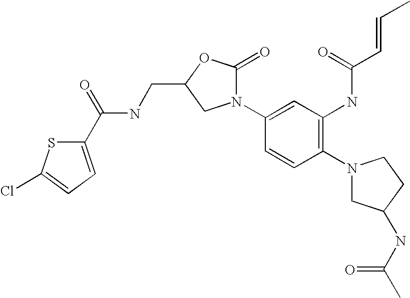 | 3.31 | 65.2 |
| 251 | 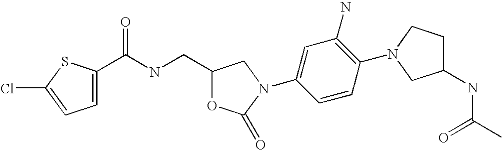 | 2.86 | 36.5 |
| 252 | 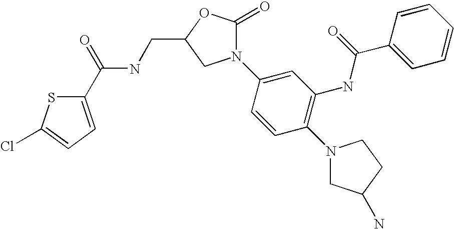 | 2.69 | 89.8 |
| 253 | 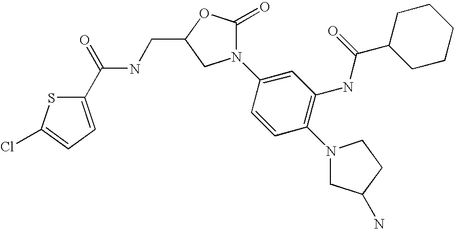 | 2.81 | 67.4 |
| 254 | 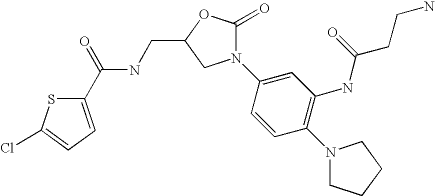 | 2.19 | 75.4 |

All products of the solid phase-assisted synthesis were characterized by means of LC-MS. For this, the following separation system was used as standard: HP 1100 with UV detector (208-400 nm), 40° C. oven temperature, Waters Symmetry C18 column (50 mm×2.1 mm, 3.5 μm), eluent A: 99.9% acetonitrile/0.1% formic acid, eluent B: 99.9% water/0.1% formic acid; gradient:

| Time | A: % | B: % | Flow |
|---|---|---|---|
| 0.00 | 10.0 | 90.0 | 0.50 |
| 4.00 | 90.0 | 10.0 | 0.50 |
| 6.00 | 90.0 | 10.0 | 0.50 |
| 6.10 | 10.0 | 90.0 | 1.00 |
| 7.50 | 10.0 | 90.0 | 0.50 |

The detection of the substances was carried out by means of a Micromass Quattro LCZ MS, ionization: ESI positive/negative.

In the structures mentioned above which contain the or the radicals

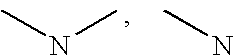

### or —O, an

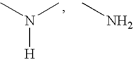

or —OH function is always meant.

1-6. (canceled)

7. A method for the control of microangiopathies in humans and animals comprising administering a therapeutically effective amount of at least one compound of the formula (I) 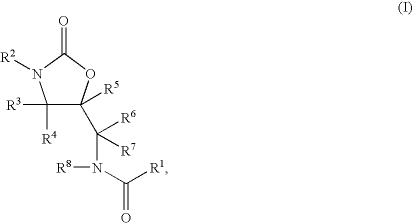
in which
R¹ represents 2-thiophene, which is substituted in the 5-position by a radical from the group consisting of chlorine, bromine, methyl or trifluoromethyl,
R² represents D-A-:
where:
the radical “A” represents phenylene;
the radical “D” represents a saturated 5- or 6-membered heterocycle,
which is linked via a nitrogen atom to “A”,
which in direct vicinity to the linking nitrogen atom has a carbonyl group and in which a ring carbon member can be replaced by a heteroatom from the series S, N and O;
where
the previously defined group “A” in the meta-position with respect to the linkage to the oxazolidinone can optionally be mono- or disubstituted by a radical from the group consisting of fluorine, chlorine, nitro, amino, trifluoromethyl, methyl or cyano,
R³, R⁴, R⁵, R⁶, R⁷ and R⁸ represent hydrogen,
or one of its salts, solvates and solvates of the salts
to a human or animal in need thereof.

8. The method of claim 7, wherein the compound of the formula (I) is 5-chloro-N-({(5S)-2-oxo-3-[4-(3-oxo-4-morpholinyl)phenyl]-1,3-oxazolidin-5-yl}methyl)-2-thiophenecarboxamide of the formula 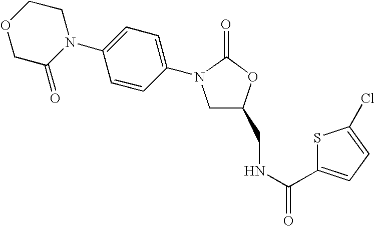
or one of its salts, solvates and solvates of the salts.

9. A method for the for the treatment and/or prophylaxis of occlusive syndromes selected from the group consisting of occlusive syndromes resulting on the skin and other organs, primary forms of thrombotic microangiopathies (TMA), secondary forms of TMA, diabetic microangiopathies, venous occlusive diseases of the liver, cerebral vasculitis and microthromboses of the placenta, and the repeated miscarriages resulting therefrom in humans and animals, the method comprising administering a therapeutically effective amount of at least one compound of the formula (I) 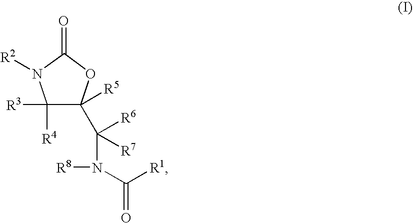
in which
R¹ represents 2-thiophene, which is substituted in the 5-position by a radical from the group consisting of chlorine, bromine, methyl or trifluoromethyl,
R² represents D-A-:
where:
the radical “A” represents phenylene;
the radical “D” represents a saturated 5- or 6-membered heterocycle,
which is linked via a nitrogen atom to “A”,
which in direct vicinity to the linking nitrogen atom has a carbonyl group and
in which a ring carbon member can be replaced by a heteroatom from the series S, N and O;
where
the previously defined group “A” in the meta-position with respect to the linkage to the oxazolidinone can optionally be mono- or disubstituted by a radical from the group consisting of fluorine, chlorine, nitro, amino, trifluoromethyl, methyl or cyano,
R³, R⁴, R⁵, R⁶, R⁷ and R⁸ represent hydrogen,
or one of its salts, solvates and solvates of the salts to a human or animal in need thereof.

10. The method of claim 9, wherein the occlusive syndrome is a primary form of a thrombotic microangiopathy selected from the group consisting of thrombotic-thrombocytopenic purpura (TTP) and hemolytic-uremic syndrome (HUS).

11. The method of claim 9, wherein the occlusive syndrome is a secondary form of a thrombotic microangiopathy arising after infections, taking of medicaments, endocarditis, collagenosis, malignant tumors, transplants and in secondary forms of TMA occurring in pregnancy.

12. The method of claim 9, wherein the occlusive syndrome is a diabetic microangiopathy selected from the group consisting of diabetic retinopathy, glomerulopathy, trophic disorders and diabetic gangrene.

13. The method of claim 9, wherein the compound of the formula (I) is 5-chloro-N-({(5S)-2-oxo-3-[4-(3-oxo-4-morpholinyl)phenyl]-1,3-oxazolidin-5-yl}methyl)-2-thiophenecarboxamide of the formula 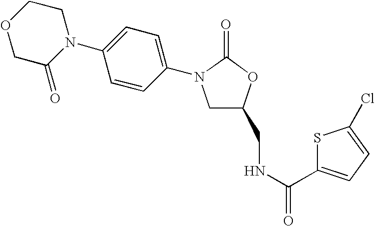
or one of its salts, solvates and solvates of the salts.

14. The method of claim 10, wherein the compound of the formula (I) is 5-chloro-N-({(5S)-2-oxo-3-[4-(3-oxo-4-morpholinyl)phenyl]-1,3-oxazolidin-5-yl}methyl)-2-thiophenecarboxamide of the formula 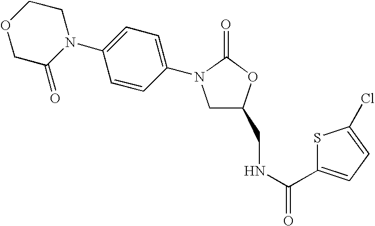
or one of its salts, solvates and solvates of the salts.

15. The method of claim 11, wherein the compound of the formula (I) is 5-chloro-N-({(5S)-2-oxo-3-[4-(3-oxo-4-morpholinyl)phenyl]-1,3-oxazolidin-5-yl}methyl)-2-thiophenecarboxamide of the formula 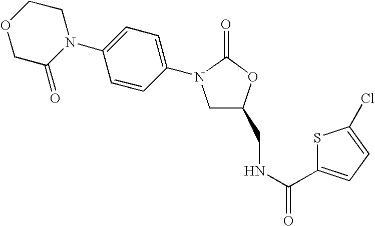
or one of its salts, solvates and solvates of the salts.

16. The method of claim 12, wherein the compound of the formula (I) is 5-chloro-N-({(5S)-2-oxo-3-[4-(3-oxo-4-morpholinyl)phenyl]-1,3-oxazolidin-5-yl}methyl)-2-thiophenecarboxamide of the formula 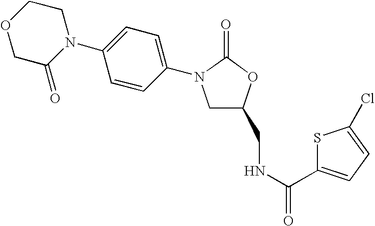
or one of its salts, solvates and solvates of the salts.

17. The method of claim 7, wherein the compound of the formula (I) is administered in the form of a medicament further comprising an inert, non-toxic, pharmaceutically suitable excipient.

18. A method for the control of harmful capillary buds resulting in the case of microangiopathies in humans and animals, the method comprising administering a therapeutically effective amount of at least one compound of the formula (I) 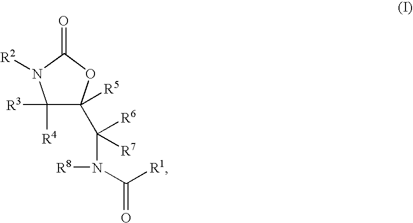
in which
R¹ represents 2-thiophene, which is substituted in the 5-position by a radical from the group consisting of chlorine, bromine, methyl or trifluoromethyl,
R² represents D-A-:
where:
the radical “A” represents phenylene;
the radical “D” represents a saturated 5- or 6-membered heterocycle,
which is linked via a nitrogen atom to “A”,
which in direct vicinity to the linking nitrogen atom has a carbonyl group and
in which a ring carbon member can be replaced by a heteroatom from the series S, N and O;
where
the previously defined group “A” in the meta-position with respect to the linkage to the oxazolidinone can optionally be mono- or disubstituted by a radical from the group consisting of fluorine, chlorine, nitro, amino, trifluoromethyl, methyl or cyano,
R³, R⁴, R⁵, R⁶, R⁷ and R⁸ represent hydrogen,
or one of its salts, solvates and solvates of the salts to a human or animal in need thereof.
